(19) United States
(12) Patent Application Publication
(10) Pub. No.: US 2012/0009141 A1
(43) Pub. Date: Jan. 12, 2012

(54) FUNCTIONALIZED PYRROLIDINES AND USE THEREOF AS IAP INHIBITORS

(75) Inventors: Alain Laurent, Montreal (CA); Melanie Proulx, Montreal (CA); James B. Jaquith, Pincourt (CA); Irina Denissova, Montreal (CA)

(73) Assignee: PHARMASCIENCE INC., MONTREAL, QC (CA)

(21) Appl. No.: 13/057,730

(22) PCT Filed: Aug. 7, 2009
(86) PCT No.: PCT/CA2009/001110

§ 371 Date: Jul. 27, 2011

Related U.S. Application Data
(60) Provisional application No. 61/136,027, filed on Aug. 7, 2008.

(51) Int. Cl. A61K 31/427; C07D 413/14; C07D 403/12; C07D 403/14; C07D 519/00; A61K 31/422; A61K 31/428; A61K 31/4025; A61K 31/4045; A61K 31/437; A61K 38/19; A61K 39/395; A61P 35/00; A61P 29/00; A61P 19/02; A61K 31/56; A61K 31/4706; A61K 31/675; A61K 38/13; A61K 38/20; C12N 5/071; C07D 417/14

(52) U.S. Cl. 424/85.2; 548/204; 548/236; 548/150; 548/156; 548/200; 548/518; 548/455; 546/121; 514/365; 514/374; 514/366; 514/367; 514/422; 514/414; 514/300; 424/85.1; 424/172.1; 514/171; 424/153.1; 424/133.1; 514/313; 514/110; 514/20.5; 435/375

(57) ABSTRACT

A compound of Formula 1: or a salt thereof, and methods for the use thereof, especially as an IAP inhibitor, as well as related compounds, compositions, and methods.

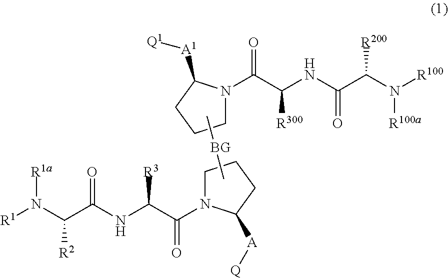

### BACKGROUND OF THE INVENTION

Apoptosis, or programmed cell death, typically occurs in the normal development and maintenance of healthy tissues in multicellular organisms. It is a complex process which results in the removal of damaged, diseased or developmentally redundant cells, in the absence of signs of inflammation or necrosis.

Intrinsic apoptotic pathways are known to be dysregulated in cancer and lymphoproliferative syndromes, as well as autoimmune disorders such as multiple sclerosis and rheumatoid arthritis, as well as in neurodegenerative diseases and inflammation. Additionally, alterations in a host apoptotic response have been described in the development or maintenance of viral and bacterial infections.

Apoptosis is the ordered dismantling of cellular components leading to cell death, which occurs as a normal part of development, the maintenance of normal cellular homeostasis, or as a consequence of injurious stimuli such as chemotherapy and radiation. Cancer cells, however, gain the ability to overcome or circumvent apoptosis and continue with inappropriate proliferation despite strong pro-apoptotic signals such as hypoxia, endogenous cytokines, radiation treatments and chemotherapy. In autoimmune disease, pathogenic effector cells can become resistant to normal apoptotic cues. Resistance results from numerous mechanisms, including alterations in the apoptotic machinery due to increased activity of anti-apoptotic pathways or expression of anti-apoptotic genes. Thus, approaches that reduce the threshold of apoptotic induction in cancer cells by overcoming innate resistance mechanisms may be of significant clinical utility.

The caspases are a family of proteolytic enzymes from the class of cysteine proteases which are known to initiate and execute apoptosis. In normal cells, the caspases are present as inactive zymogens, which are catalytically activated following external signals, for example those resulting from ligand driven Death Receptor activation, such as cytokines or immunological agents, or by release of mitochondrial factors, such as cytochrome C following genotoxic, chemotoxic, or radiation-induced cellular injury. The Inhibitors of Apoptosis Proteins (IAPs) constitute a family of proteins which are capable of binding to and inhibiting the caspases, thereby suppressing cellular apoptosis. Because of their central role in regulating caspase activity, the IAPs are capable of inhibiting programmed cell death from a wide variety of triggers, which include loss of homeostatic, or endogenous cellular growth control mechanisms, as well as chemotherapeutic drugs and irradiation.

The IAPs contain one to three homologous structural domains known as baculovirus IAP repeat (BIR) domains. They may also contain a RING zinc finger domain at the C-terminus, with a capability of inducing ubiquitinylation of IAP-binding molecules via its E3 ligase function. The human IAPs, XIAP, HIAP1 (also referred to as cIAP2), and HIAP2 (cIAP1) each have three BIR domains, and a carboxy terminal RING zinc finger. Another IAP, NAIP, has three BIR domains (BIR1, BIR2 and BIR3), but no RING domain, whereas Livin, TsIAP and MLIAP have a single BIR domain and a RING domain. The X chromosome-linked inhibitor of apoptosis (XIAP) is an example of an IAP which can inhibit the initiator caspase, known as caspase-9, and the effector caspases, Caspase-3 and Caspase-7, by direct binding. It can also induce the removal of caspases through the ubiquitylation-mediated proteasome pathway via the E3 ligase activity of a RING zinc finger domain. It is via the BIR3 domain that XIAP binds to and inhibits caspase-9. The linker-BIR2 domain of XIAP inhibits the activity of caspases-3 and -7. The BIR domains have also been associated with the interactions of IAPs with tumor necrosis factor-receptor associated factor (TRAFs)-1 and -2, and to TAB1, as adaptor proteins effecting survival signaling through NFkB activation. The IAPs thus function as a direct brake on the apoptosis cascade, by preventing the action of, or inhibiting active caspases and by re-directing cellular signaling to a pro-survival mode.

Cancer cells and cells involved in autoimmune disease may avoid apoptosis by the sustained over-expression of one or more members of the IAP family of proteins. For example, IAP overexpression has been demonstrated to be prognostic of poor clinical outcome in multiple cancers, and decreased IAP expression through RNA antisense or siRNA strategies sensitizes tumor cells to a wide variety of apoptotic insults including chemotherapy, radiotherapy and death receptor ligands. For XIAP this is shown in cancers as diverse as leukemia and ovarian cancer. Over expression of HIAP1 and HIAP2 resulting from the frequent chromosome amplification of the 11q21-q23 region, which encompasses both, has been observed in a variety of malignancies, including medulloblastomas, renal cell carcinomas, glioblastomas, and gastric carcinomas. Also, abnormally apoptotic resistant T-cells have been demonstrated in autoimmune diseases such as multiple sclerosis, rheumatoid arthritis, idiopathic thrombocytopenic purpura, and alopecia areata. Other abnormally apoptotic resistant cells also have been linked to autoimmune disease, such as fibroblast-like synoviocytes in rheumatoid arthritis (RA) and keratinocytes in psoriasis. Thus, IAPs are valid therapeutic targets and compounds that inhibit their expression or function may have significant utility in the treatment of proliferative diseases associated with dysregulated apoptosis, including cancer and autoimmune diseases.

### SUMMARY OF THE INVENTION

In one embodiment of the present invention, there is provided a compound of Formula 1:

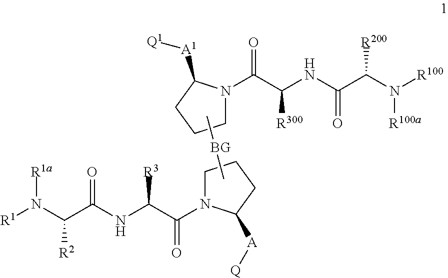

or a salt thereof, wherein
n is 0, 1, or 2;
m is 0, 1 or 2;

### Y is NH, O or S;

### BG is —X-L-X¹—;

X and X¹ are independently

1) O,

2) NR¹¹,

3) S,

4) —C₁-C₆ alkyl-,

5) —C₁-C₆ alkyl-O—,

6) —C₁-C₆ alkyl-NR¹¹—,

7) —C₁-C₆ alkyl-S—,

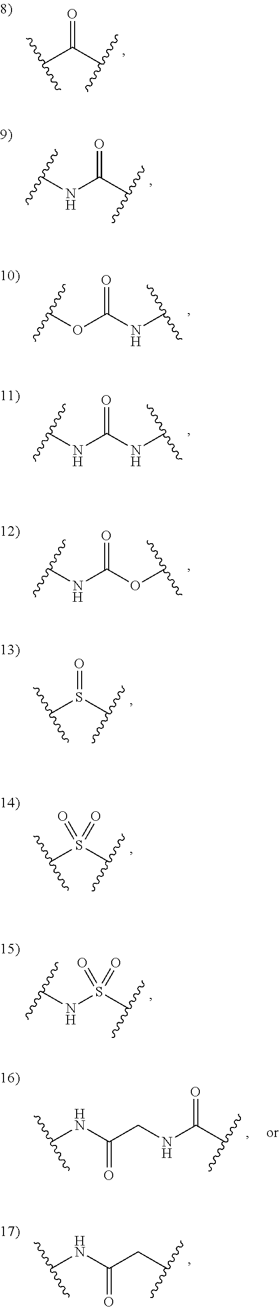

L is

1) —C₁-C₂₀ alkyl-,

2) —C₂-C₆ alkenyl-,

3) —C₂-C₄ alkynyl-,

4) —C₃-C₇, cycloalkyl-,

5) -aryl-,

6) -biphenyl-,

7) -heteroaryl-,

8) -biheteroaryl-,

9) -heterocyclyl-,

10) —C₁-C₆ alkyl-(C₂-C₆ alkenyl)-C₁-C₆ alkyl-,

11) —C₁-C₆ alkyl-(C₂-C₄ alkynyl)-C₁-C₆ alkyl-,

12) —C₁-C₆ alkyl-(C₃-C₇ cycloalkyl)-C₁-C₆ alkyl-,

13) —C₁-C₆ alkyl-aryl-C₁-C₆ alkyl-,

14) —C₁-C₆ alkyl-biphenyl-C₁-C₆ alkyl-,

15) —C₁-C₆ alkyl-heteroaryl-C₁-C₆ alkyl-,

16) —C₁-C₆ alkyl-heterocycyl-C₁-C₆ alkyl-,

17) —C₁-C₆ alkyl-Y—C₁-C₆ alkyl-,

18) -aryl-Y-aryl-,

19) -heteroaryl-Y-heteroaryl-,

20) -heterocyclyl-Y-heterocyclyl-,

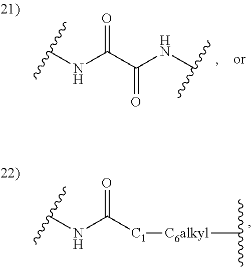

wherein the alkyl, alkenyl, alkynyl, and cycloalkyl are optionally substituted with one or more R⁴ substituents, and the aryl, biphenyl, heteroaryl, and heterocyclyl are optionally substituted with one or more R⁸ substituents;

A and A¹ are independently

1) —(CH₂)ₙ—,

2) —CH(C₁-C₆ alkyl)-,

3) —CH(C₃-C₇ cycloalkyl)-,

4) —C₃-C₇ cycloalkyl-, or

5) —CH(C₁-C₆ alkyl-C₃-C₇ cycloalkyl)-;

Q and Q¹ are independently

1) aryl,

2) heteroaryl,

3) heterocyclyl,

4) heterobicyclyl,

5) —X²-aryl,

6) —X²-heteroaryl;

7) —X²-heterocyclyl, or

8) —X²-heterobicyclyl,

wherein the aryl, heteroaryl, heterocyclyl, and heterobicyclyl are optionally substituted with one or more R⁸ substituents;

X² is

1) —O—,

2) —NR¹¹—,

3) —S(O)ₘ—, or

4) —C(O)—;

R¹, R^{1a}, R¹⁰⁰ and R^{100a} are independently

1) H, or

2) C₁-C₆ alkyl optionally substituted with one or more R⁴ substituents;

R² and R²⁰⁰ are independently

1) H, or

2) C₁-C₆ alkyl optionally substituted with one or more R⁴ substituents;

R³ and R³⁰⁰ are independently

1) H,

2) C₁-C₆ alkyl,

3) C₃-C₇ cycloalkyl,

4) C₃-C₇ cycloalkenyl,

5) aryl,

6) biphenyl,

7) heteroaryl,

8) heterocyclyl, or

9) heterobicyclyl,

wherein the alkyl, cycloalkyl, and cycloalkenyl are optionally substituted with one or more R⁴ substituents; and wherein the aryl, biphenyl, heteroaryl, and heterobicyclyl are optionally substituted with one of more R⁸ substituents;

R⁴ is

1) halogen,

2) NO₂,

3) CN,

4) haloalkyl,

5) C₁-C₆ alkyl,

6) C₂-C₆ alkenyl,

7) C₂-C₄ alkynyl,

8) C₃-C₇ cycloalkyl,

9) C₃-C₇ cycloalkenyl,

10) aryl,

11) heteroaryl,

12) heterocyclyl,

13) heterobicyclyl,

14) —OR⁵,

15) —S(O)ₘR⁵,

16) —NR⁶R⁷,

17) —NR⁶S(O)₂R⁹,

18) —C(O)R⁵,

19) —C(O)OR⁵,

20) —CONR⁶R⁷,

21) —S(O)₂NR⁶R⁷

22) —OC(O)R⁵,

23) —OC(O)Y—R⁹,

24) —SC(O)R⁵, or

25) —NC(Y)NR⁶R⁷,

wherein the aryl, heteroaryl, heterocyclyl, and heterobicyclyl are optionally substituted with one or more R⁸ substituents;

R⁵ is

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) R⁶R⁷NC(═Y),

13) C₁-C₆ alkyl-C₂-C₄ alkenyl, or

14) C₁-C₆ alkyl-C₂-C₄ alkynyl,

wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;

R⁶ and R⁷ are each independently

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) —C(O)R⁹,

13) —C(O)Y—R⁹, or

14) —S(O)₂—R⁹,

wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;
or R⁶ and R⁷ together with the nitrogen atom to which they are bonded form a five, six or seven membered heterocyclic ring optionally substituted with one or more R⁴ substituents;

R⁸ is

1) halogen,

2) NO₂,

3) CN,

4) C₁-C₆ alkyl,

5) C₂-C₆ alkenyl,

6) C₂-C₄ alkynyl,

7) C₃-C₇ cycloalkyl,

8) C₃-C₇ cycloalkenyl,

9) haloalkyl,

10) —OR⁵,

11) —NR⁶R⁷,

12) —SR⁵,

13) —C(O)R⁵,

14) —C(O)OR⁵,

15) —S(O)ₘR⁶,

16) —CONR⁶R⁷,

17) —S(O)₂NR⁶R⁷,

18) aryl,

19) heteroaryl,

20) heterocyclyl, or

21) heterobicyclyl,

wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;

R⁹ is

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl, or

10) heterobicyclyl,

wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;

R¹⁰ is

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl,

10) heterobicyclyl,

11) C(O)—R⁹,

12) C(O)O—R⁹,

13) C(O)NR⁶R⁷,

14) S(O)ₘ—R⁹, or

15) C(═Y)NR⁸R⁷,

wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;

In another aspect of the present invention, there is provided an intermediate compound represented by Formula 2:

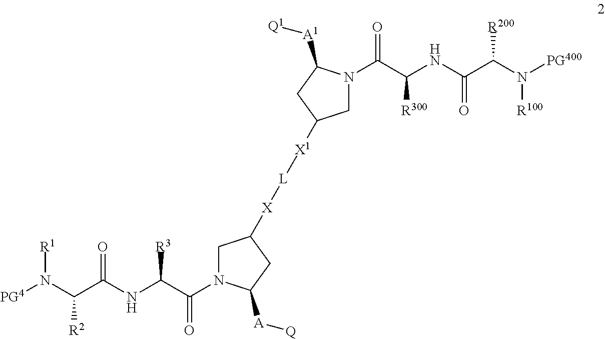

wherein PG⁴ and PG⁴⁰⁰ are suitable protecting groups, and L, X, X¹, R³, R³⁰⁰, R², R²⁰⁰, R¹, R¹⁰⁰, A, A¹, Q and Q¹ are as defined herein.

In another aspect of the present invention, there is provided an intermediate compound represented by Formula 3:

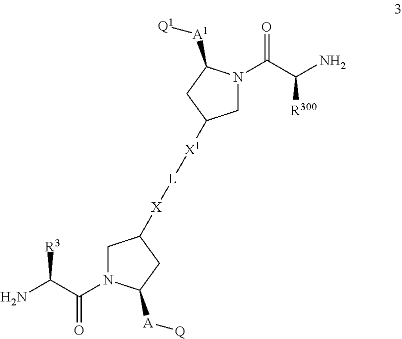

wherein L, X, X¹, R³, R³⁰⁰, A, A¹, Q and Q¹ are as defined herein.

In another aspect of the present invention, there is provided an intermediate compound represented by Formula 4:

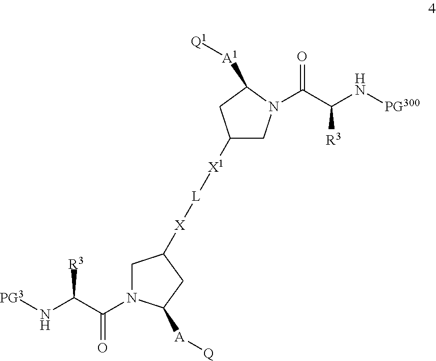

wherein PG³ and PG³⁰⁰ are suitable protecting groups, and L, X, X¹, R³, R³⁰⁰, A, A¹, Q and Q¹ are as defined herein.

In another aspect of the present invention, there is provided an intermediate compound represented by Formula 5:

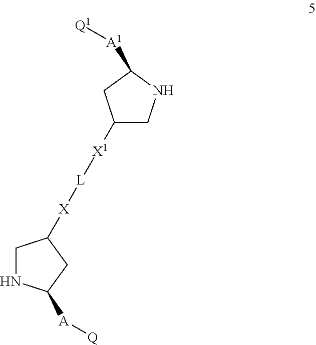

wherein L, X, X¹, A, A¹, Q and Q¹ are as defined herein.

In another aspect of the present invention, there is provided an intermediate compound represented by Formula 6:

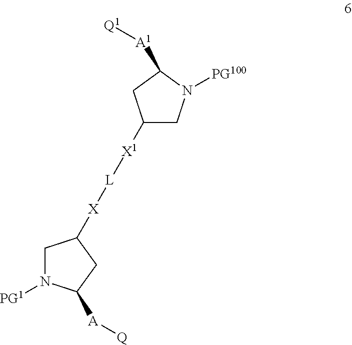

wherein PG¹ and PG¹⁰⁰ are suitable protecting groups, and L, X, X¹, A, A¹, Q and Q¹ are as defined herein.

In another aspect of the present invention, there is provided an intermediate compound represented by Formula 7:

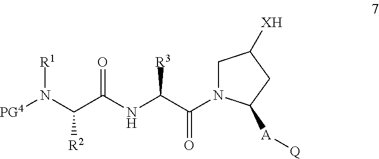

wherein PG⁴ is a suitable protecting group, and X, R³, R², R¹, A and Q are as defined herein.

In another aspect of the present invention, there is provided an intermediate compound represented by Formula 8:

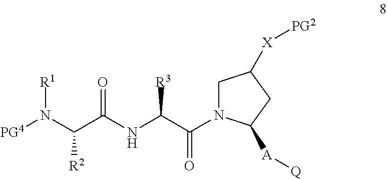

wherein PG² and PG⁴ are suitable protecting groups, and X, R³, R², R¹, A and Q are as defined herein.

In another aspect of the present invention, there is provided an intermediate compound represented by Formula 9:

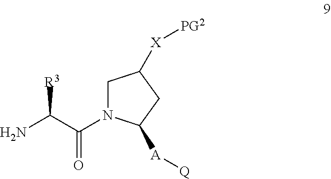

wherein PG² is a suitable protecting group, and X, R³, A and Q are as defined herein.

In another aspect of the present invention, there is provided an intermediate compound represented by Formula 10:

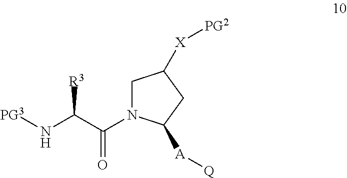

wherein PG³ and PG² are suitable protecting groups, and X, R³, A and Q are as defined herein.

In another aspect of the present invention, there is provided an intermediate compound represented by Formula 11:

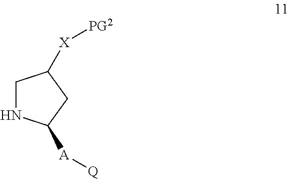

wherein PG² is a suitable protecting group, and X, A and Q are as defined herein.

In another aspect of the present invention, there is provided an intermediate compound represented by Formula 12:

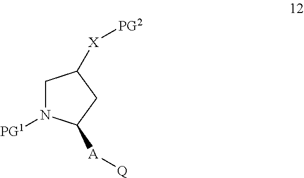

wherein PG² and PG¹ are suitable protecting groups, and X, A and Q are as defined herein.

In another aspect of the present invention, there is provided a method for the preparation of a pharmaceutically acceptable salt of a compound of Formula 1. The method can comprise treating a compound of the invention with a pharmaceutically acceptable acid (e.g., 1 to 2 equivalents of a pharmaceutically acceptable acid), so as to form a pharmaceutically acceptable salt of a compound of Formula 1.

In another aspect of the present invention, there is provided a pharmaceutical composition comprising a compound of Formula 1 and a pharmaceutically acceptable carrier, diluent or excipient, as well as a method of preparing same comprising combining a compound of Formula 1 with a pharmaceutically acceptable carrier, diluents, or excipient.

In another aspect of the present invention, there is provided a method of treating a proliferative disorder or a disease state characterized by insufficient apoptosis, the method comprising: administering to a subject in need thereof, a therapeutically effective amount of a compound or pharmaceutical composition, as described above, so as to treat the proliferative disorder or disease state.

In another aspect of the present invention, there is provided a method of modulating IAP function, the method comprising: contacting a cell with a compound of the present invention so as to prevent binding of a BIR binding protein to an IAP BIR domain thereby modulating the IAP function.

In another aspect of the present invention, there is provided a probe, the probe being a compound of Formula 1 labeled with a detectable label or an affinity tag. In other words, the probe comprises a compound of Formula 1 and a detectable label.

In another aspect of the present invention, there is provided a method of identifying compounds that bind to an IAP BIR domain, the assay comprising:
a) contacting an IAP BIR domain with a probe, as described herein, to form a probe:BIR domain complex, the probe being displaceable by a test compound;
b) measuring a signal from the probe so as to establish a reference level;
c) incubating the probe:BIR domain complex with the test compound;
d) measuring the signal from the probe; and
e) comparing the signal from step d) with the reference level, a modulation of the signal (e.g., an increase or decrease in the signal relative to the reference level) being an indication that the test compound binds to the BIR domain.

In another aspect of the present invention, there is provided a method of detecting loss of function or suppression of IAPs in vivo, the method comprising: a) administering to a subject, a therapeutically effective amount of a pharmaceutical composition, as defined above; b) isolating a tissue sample from the subject; and c) detecting a loss of function or suppression of IAPs from the sample.

### DETAILED DESCRIPTION OF THE INVENTION

Provided herein is a compound of Formula 1:

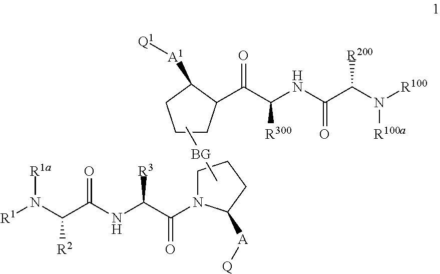

or a salt thereof. Compounds of Formula 1 also can be represented by the following formula, in which M1 and M2 represent independent BIR binding domains:

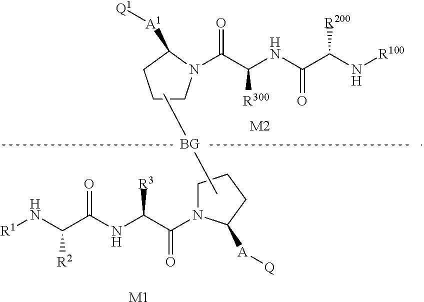

In one subset of compounds of Formula 1, M1 is the same as M2 and the dotted line denotes a line of symmetry. In another subset, M1 is different from M2.

One skilled in the art will recognize that when M1 and M2 are the same, the substituents in M1 (e.g., R¹, R², R³, R⁶, R⁷, R⁸, R⁹, R¹⁰, R¹¹, R¹², n, m, Y, A, and Q) have the same meaning as the corresponding substituents in M2 (e.g., R¹⁰⁰, R²⁰⁰, R³⁰⁰, R⁶, R⁷, R⁸, R⁹, R¹⁰, R¹¹, R¹², n, m, Y, A¹, and Q¹). When M1 and M2 are different, at least one of the foregoing substituents in M1 is different from the corresponding substituent in M2.

In a preferred aspect, the compounds of Formula 1 are useful as BIR domain binding compounds in mammalian IAPs. The following embodiments further illustrate compounds of Formula 1.

### Core:

The compounds of Formula 1 can have any configuration about the core. For example, such compounds can include compounds of any of Formulas 1A, 1B, and 1C:

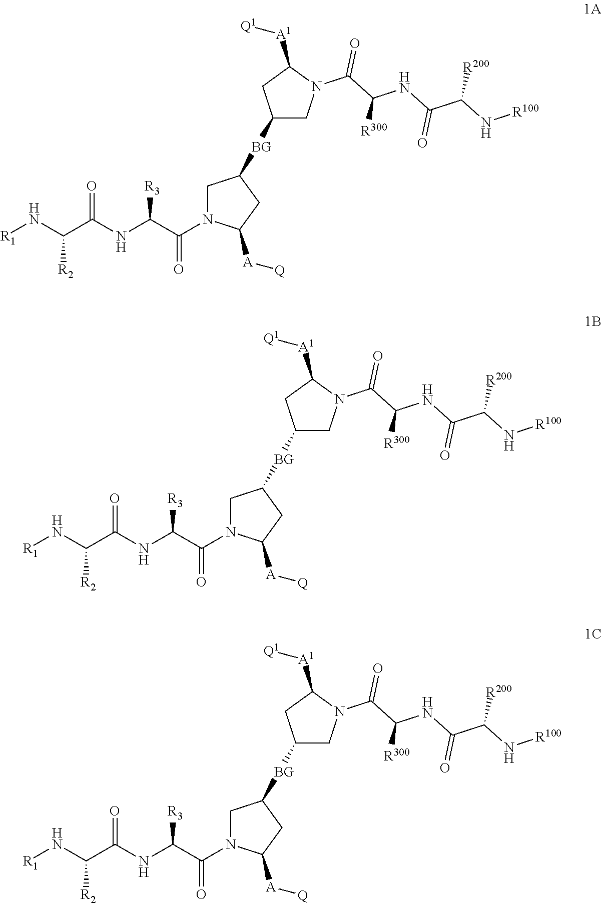

wherein BG, A, A¹, Q, Q¹, R¹, R¹⁰⁰, R², R²⁰⁰, R³, and R³⁰⁰ are as defined hereinabove and hereinafter.

### BG:

According to Formula 1, BG is —X-L-X¹—. Thus, compounds of Formula 1 include compounds of Formula 1a through 1c:

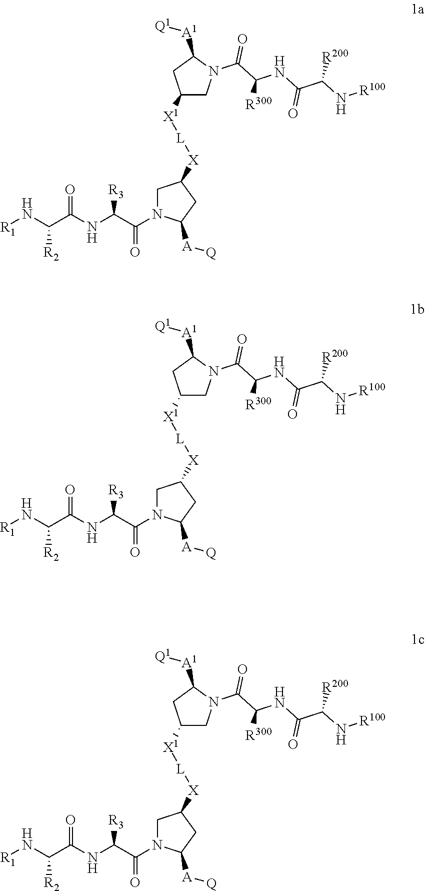

wherein L, X, X¹, A, A¹, Q, Q¹, R¹, R¹⁰⁰, R², R²⁰⁰, R³, and R³⁰⁰ are as defined hereinabove and hereinafter.

### X and X¹:

X and X¹ can be any moiety as previously defined. In one subset of the aforesaid compounds, X and X¹ are independently selected from

1) O,

2) NR¹²,

3) S,

4) —C₁-C₆ alkyl-,

5) —C₁-C₆ alkyl-O—,

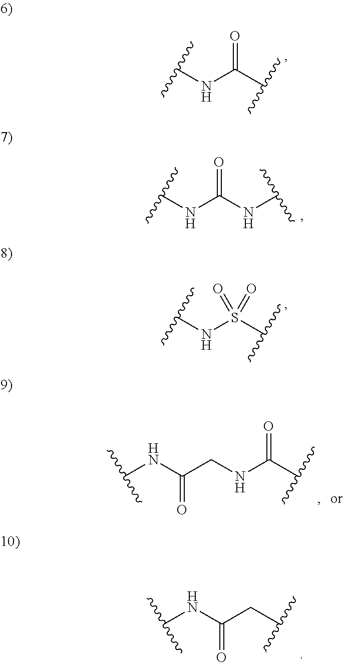

Any and each individual definition of X and X¹ as set out herein may be combined with any and each individual definition of Core, L, A, A¹, R¹, R², R¹⁰⁰, R²⁰⁰, R³, R³⁰⁰, Q or Q¹ as set out herein.

### L:

L can be any moiety as previously defined. In one subset of the aforesaid compounds, L is:

1) —C₁-C₂₀ alkyl-,

2) —C₃-C₇ cycloalkyl-,

3) -aryl-,

4) -biphenyl-,

5) -heteroaryl-,

6) —C₁-C₆ alkyl-(C₂-C₄ alkynyl)-C₁-C₆ alkyl-

7) —C₁-C₆ alkyl-aryl-C₁-C₆ alkyl-,

8) —C₁-C₆ alkyl-biphenyl-C₁-C₆ alkyl-,

9) —C₁-C₆ alkyl-heteroaryl-C₁-C₆ alkyl-,

10) —C₁-C₆ alkyl-heterocycyl-C₁-C₆ alkyl-,

11) —C₁-C₆ alkyl-Y—C₁-C₆ alkyl-,

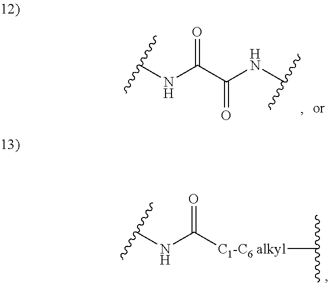

wherein the alkyl, the alkenyl, the alkynyl, the cycloalkyl and the heterocyclyl are optionally substituted with one or more R⁴ substituents, and the aryl, biphenyl and heteroaryl are optionally substituted with one or more R⁸ substituents. By way of further illustration, L can be:

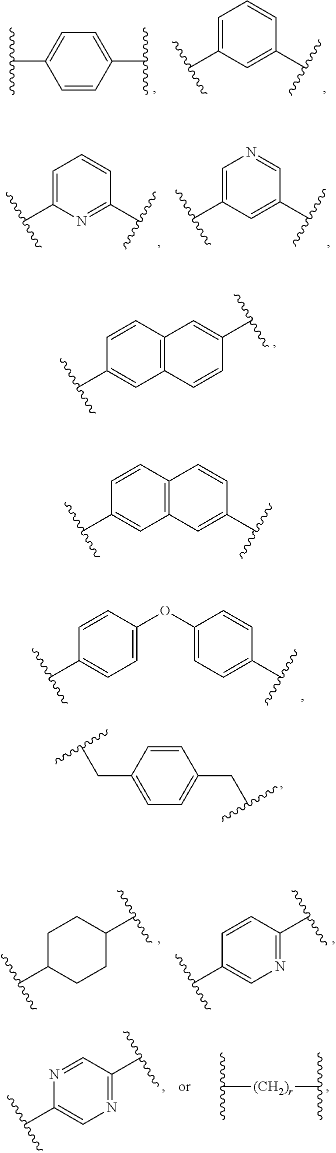

wherein r is 1, 2, 3, 4, 5, 6, 7, 8, 9, or 10.

Any and each individual definition of L as set out herein may be combined with any and each individual definition of Core, A, A¹, R¹, R², R¹⁰⁰, R²⁰⁰, R³, R³⁰⁰, X, X¹, Q, or Q¹, as set out herein.

By way of further illustration, the compound of Formula 1 can be a compound of Formula 1.1 through 1.9:

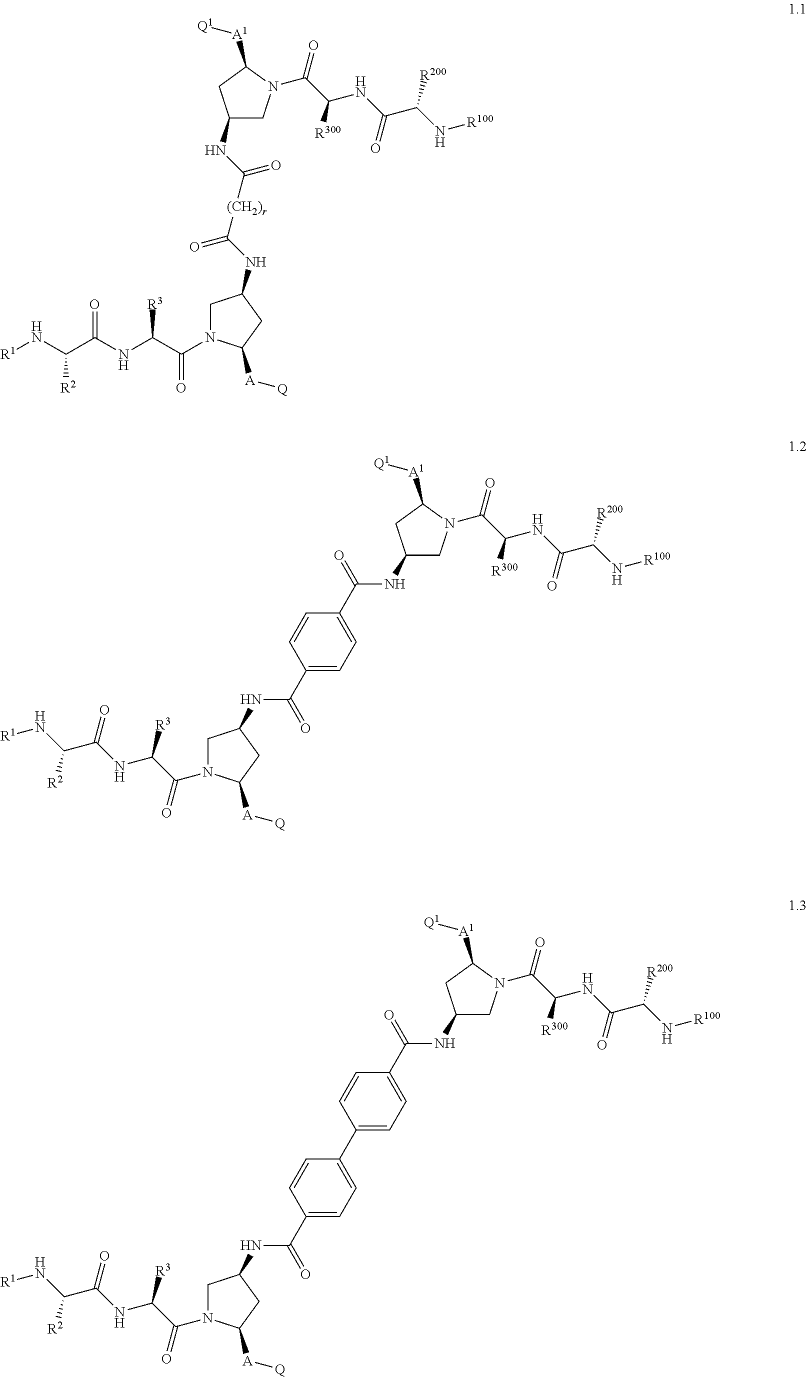

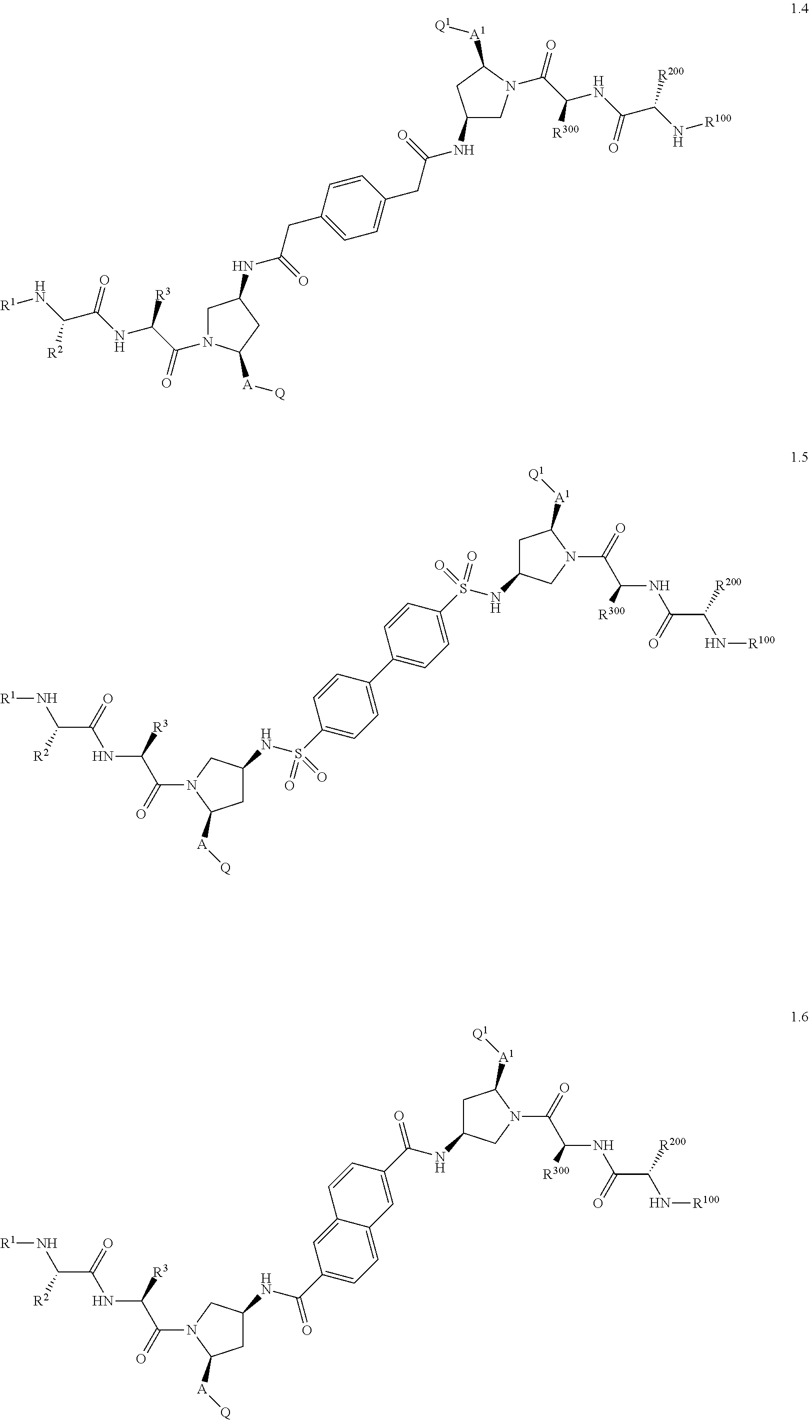

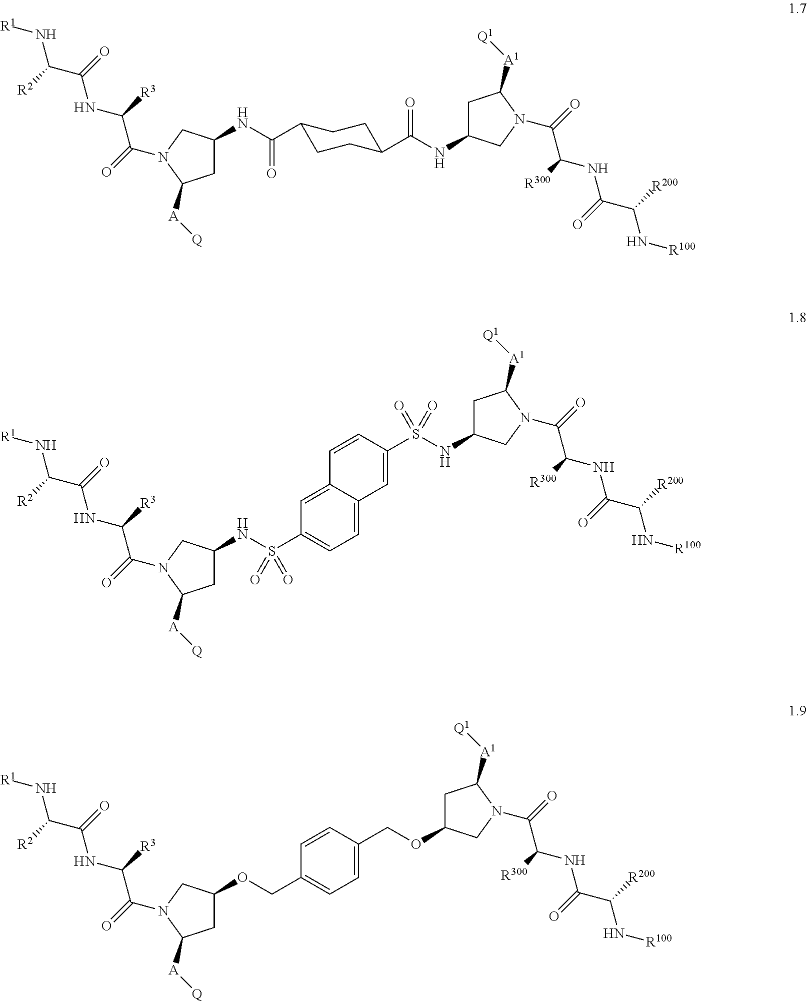

wherein r is 1, 2, 3, 4, 5, 6, 7, 8, 9, or 10, and A, A¹, Q, Q¹, R¹, R¹⁰⁰, R², R²⁰⁰, R³ and R³⁰⁰ are as defined hereinabove or hereinafter.

### R¹ and R¹⁰⁰:

R¹ and R¹⁰⁰ can be any moiety as previously defined. In one subset of the aforesaid compounds, R¹ and R¹⁰⁰ are both H. In another subset of the aforesaid compounds, R¹ and R¹⁰⁰ are both C₁-C₆ alkyl, or C₁-C₃ alkyl, for example, CH₃.

Any and each individual definition of R¹ and R¹⁰⁰ as set out herein may be combined with any and each individual definition of Core, A, A¹, R², R²⁰⁰, R³, R³⁰⁰, Q, Q¹, and BG as set out herein.

### R² and R²⁰⁰:

R² and R²⁰⁰ can be any moiety as previously defined. In one subset of the aforesaid compounds R² and R²⁰⁰ are both C₁-C₆ alkyl, or C₁-C₃ alkyl, for example, CH₃.

Any and each individual definition of R² and R²⁰⁰ as set out herein may be combined with any and each individual definition of Core, A, A¹, R¹, R¹⁰⁰, R³, R³⁰⁰, Q, Q¹ and BG as set out herein.

### R³ and R³⁰⁰:

R³ and R³⁰⁰ can be any moiety as previously defined. Specific examples of R³ and R³⁰⁰ include:

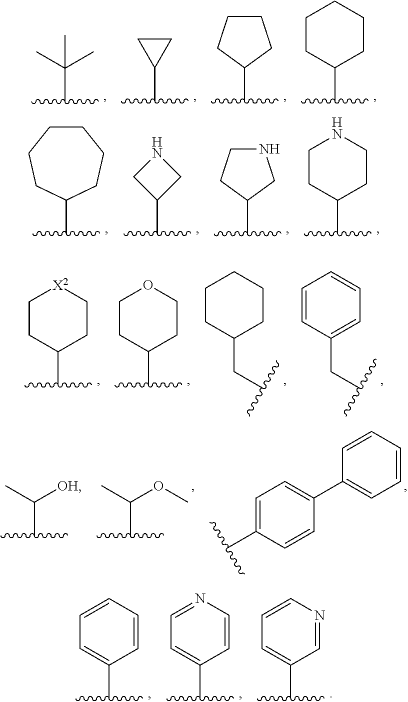

In one subset of the aforesaid compounds, R³ and R³⁰⁰ are both C₁-C₆ alkyl, optionally substituted with one or more R⁴ substituents (e.g., —C(CH₃)₃) In another subset of compounds, R³ and R³⁰⁰ are both C₃-C₇ cycloalkyl or heterocyclyl, optionally substituted with one or more R⁸ substituents.

Any and each individual definition of R³ and R³⁰⁰ as set out herein may be combined with any and each individual definition of Core, A, A¹, R¹, R¹⁰⁰, R², R²⁰⁰, Q, Q¹, and BG as set out herein.

### A and A¹

A and A¹ can be any moiety as previously defined. In one subset of compounds of Formula 1, A and A¹ are both —(CH₂)ₙ— with n=0. In an alternative subset of compounds of Formula 1, A and A¹ are both —(CH₂)ₙ— with n=1.

### Q and Q¹:

Q and Q¹ can be any moiety as previously defined. Specific examples of Q and Q¹ include the following, wherein R⁸ and R^{8′} are optional, and R^{8′} illustrates the substitution by a second R⁸ substituent group that can be the same or different from R⁸:

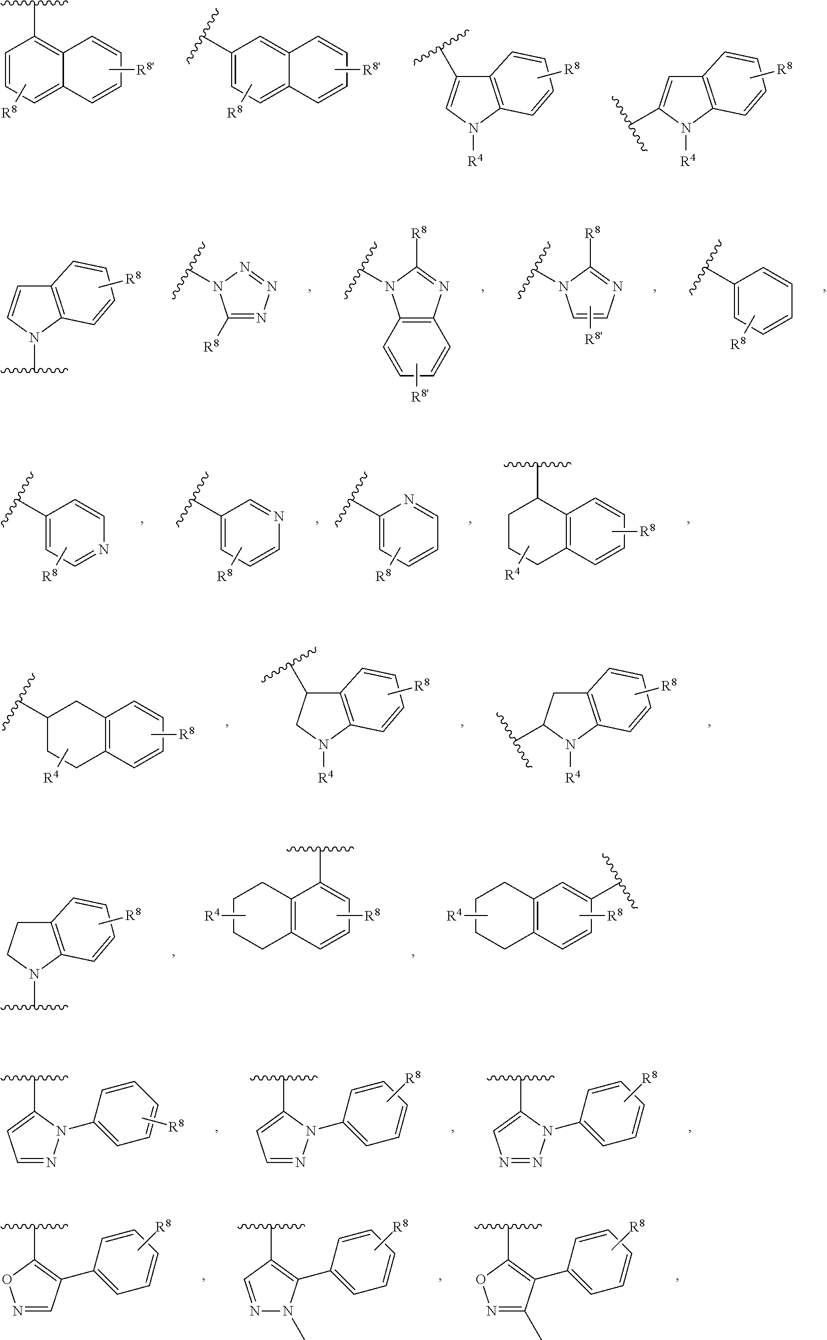

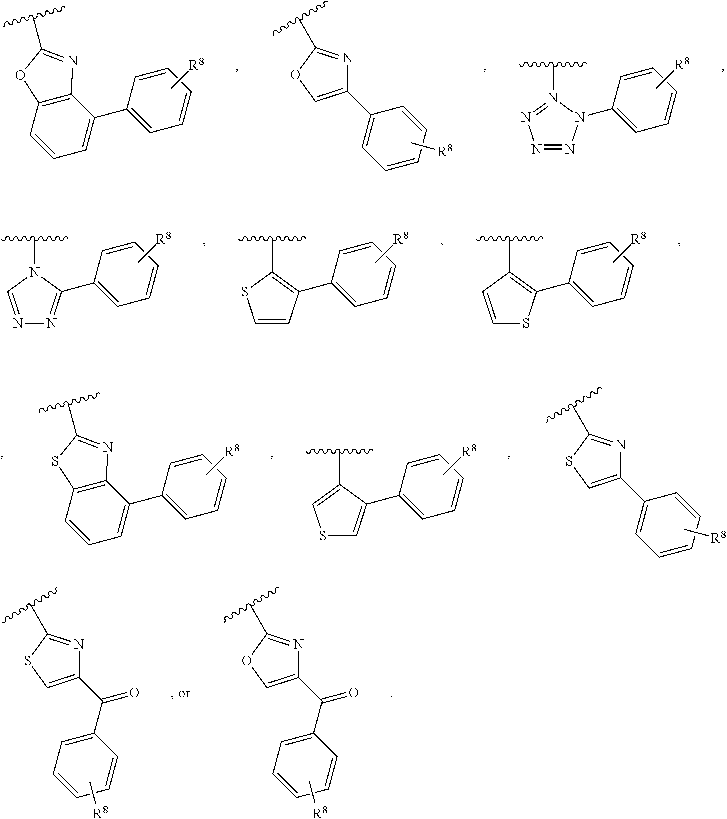

Other specific examples of Q and Q¹ include:

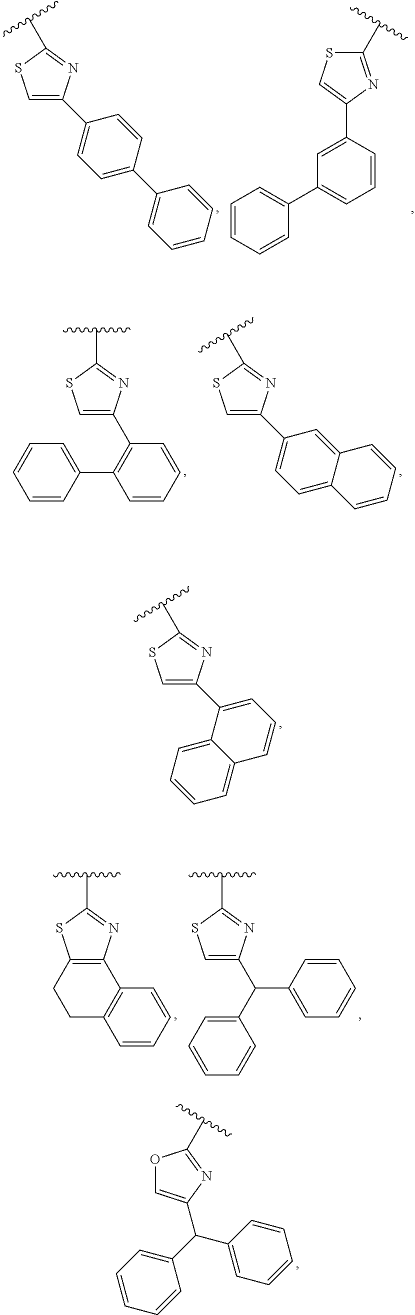

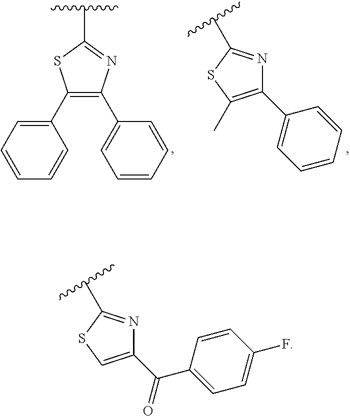

When Q is

—X²-aryl,

—X²-heteroaryl;

—X²-heterocyclyl, or

—X²-heterobicyclyl,

X² can be an group as previously defined, for example, —O—, —S(O)ₘ—, or —C(O)—.

Any and each individual definition of Q and Q¹ as set out herein may be combined with any and each individual definition of Core, A, A¹, R¹, R¹⁰⁰, R², R²⁰⁰, R³, R³⁰⁰ and BG as set out herein.

### R⁴:

R⁴ can be any moiety as previously defined. In one subset of the aforesaid compounds, R⁴ is

1) halogen,

2) NO₂,

3) CN,

4) aryl,

5) heteroaryl,

6) heterocyclyl,

7) heterobicyclyl,

8) —OR⁵,

9) —SR⁵, or

10) —NR⁶R⁷,

wherein the aryl, heteroaryl, heterocyclyl, and heterobicyclyl is optionally substituted with one or more R⁸ substituents.

Any and each individual definition of R⁴ as set out herein may be combined with any and each individual definition of Core, A, A¹, R¹, R¹⁰⁰, R², R²⁰⁰, R³, R³⁰⁰, and BG as set out herein.

### R⁶ and R⁷:

R⁶ and R⁷ can be any moiety as previously defined. In one subset of the aforesaid compounds, R⁶ and R⁷ are each independently

1) H,

2) haloalkyl,

3) C₁-C₆ alkyl,

4) C₂-C₆ alkenyl,

5) C₂-C₄ alkynyl,

6) C₃-C₇ cycloalkyl, or

7) C₃-C₇ cycloalkenyl,

wherein the alkyl, alkenyl, alkynyl, cycloalkyl, cycloalkenyl is optionally substituted with one or more R⁴ substituents.

Any and each individual definition of R⁶ and R⁷ as set out herein may be combined with any and each individual definition of Core, A, A¹, R¹, R¹⁰⁰, R², R²⁰⁰, R³, R³⁰⁰, R⁴, R⁵ and BG as set out herein.

### R⁸:

R⁸ can be any moiety as previously defined. In one aspect of the aforesaid compounds, R⁸ is

1) halogen,

2) NO₂,

3) CN,

4) haloalkyl,

5) —OR⁵,

6) —NR⁶R⁷, or

7) —SR⁵.

In another aspect of the invention, R⁸ is —COR⁵, or aryl or heteroaryl, optionally substituted with one or more additional R⁸ substituents, such as another aryl or heteroaryl group.

Any and each individual definition of R⁸ as set out herein may be combined with any and each individual definition of Core, A, A¹, R¹, R¹⁰⁰, R², R²⁰⁰, R³, R³⁰⁰, R⁴, R⁵, and BG as set out herein.

### R⁹:

R⁹ can be any moiety as previously defined. In one aspect of the aforesaid compounds, R⁹ is

1) haloalkyl,

2) C₁-C₆ alkyl,

3) C₃-C₇ cycloalkyl,

4) C₃-C₇ cycloalkenyl,

5) aryl,

6) heteroaryl,

7) heterocyclyl, or

8) heterobicyclyl,

wherein the alkyl, alkenyl, alkynyl, cycloalkyl, cycloalkenyl is optionally substituted with one or more R⁴ substituents; and wherein the aryl, heteroaryl, heterocyclyl, and heterobicyclyl is optionally substituted with one or more R⁸ substituents.

Any and each individual definition of R⁹ as set out herein may be combined with any and each individual definition of Core, A, A¹, R¹, R¹⁰⁰, R², R²⁰⁰, R³, R³⁰⁰, R⁴, R⁵, and BG as set out herein.

### R¹⁰:

R¹⁰ can be any moiety as previously defined. In one aspect of the aforesaid compounds, R¹⁰ is

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl,

10) heterobicyclyl,

11) —C(O)—R⁹,

12) —C(O)O—R⁹,

13) —C(O)NR⁶R⁷,

14) —S(O)ₘ—R⁹, or

15) —C(═Y)NR⁶R⁷,

wherein the alkyl, alkenyl, alkynyl, cycloalkyl, cycloalkenyl is optionally substituted with one or more R⁴ substituents; and wherein the aryl, heteroaryl, heterocyclyl, and heterobicyclyl is optionally substituted with one or more R⁸ substituents.

Any and each individual definition of R¹⁰ as set out herein may be combined with any and each individual definition of the other substituent groups as set out herein.

If any variable, such as R⁶, R⁶⁰⁰, R¹⁰, R¹⁰⁰⁰ and the like, occurs more than one time in any constituent structure, the definition of the variable at each occurrence is independent at every other occurrence. If a substituent is itself substituted with one or more substituents, it is to be understood that that the one or more substituents may be attached to the same carbon atom or different carbon atoms. Combinations of substituents and variables defined herein are allowed only if they produce chemically stable compounds.

One skilled in the art will understand that substitution patterns and substituents on compounds of the present invention may be selected to provide compounds that are chemically stable and can be readily synthesized using the chemistry set forth in the examples and chemistry techniques well known in the art using readily available starting materials.

It is to be understood that many substituents or groups described herein have functional group equivalents, which means that the group or substituent may be replaced by another group or substituent that has similar electronic, hybridization or bonding properties.

### DEFINITIONS

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are included but that other elements are optional and may or may not be present.

As used herein, the term “consisting of” is intended to mean including and limited to whatever follows the phrase “consisting of.” Thus the phrase “consisting of” indicates that the listed elements are included and that no other elements may be present.

As used herein, the term “alkyl” is intended to include both branched and straight chain saturated aliphatic hydrocarbon groups having the specified number of carbon atoms, for example, C₁-C₆ as in C₁-C₆— alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement, and for example, C₁-C₂₀ as in C₁-C₂₀— alkyl is defined as including groups having 1, 2, 3, 4, 5, 6, 7, 8, 9, 10, 11, 12, 13, 14, 15, 16, 17, 18, 19 or 20 carbons in a linear or branched arrangement, Examples of C₁-C₆-alkyl and C₁-C₄ alkyl as defined above include, but are not limited to, methyl, ethyl, n-propyl, i-propyl, n-butyl, t-butyl, butyl, pentyl and hexyl. For the purposes of describing the invention, the term “alkyl” encompasses an “alkylene.”

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regeochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like. For the purposes of describing the invention, the term “alkenyl” encompasses an “alkenylene.”

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alkynyls include ethynyl, 1-propynyl, 2-propynyl and the like. For the purposes of describing the invention, the term “alkynyl” encompasses an “alkynylene.”

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl. For the purposes of describing the invention, the term “cycloalkyl” encompasses a “cycloalkylene.”

As used herein, the term “cycloalkenyl” is intended to mean a monocyclic unsaturated aliphatic hydrocarbon group having the specified number of carbon atoms therein, for example, C₃-C₇ as in C₃-C₇ cycloalkenyl is defined as including groups having 3, 4, 5, 6, or 7 carbons in a monocyclic arrangement. Examples of C₃-C₇ cycloalkenyl as defined above include, but are not limited to, cyclopentenyl, and cyclohexenyl. For the purposes of describing the invention, the term “cycloalkenyl” encompasses a “cycloalkenylene.”

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second or a third 5- or 6-membered carbocyclic group which may be aromatic, saturated or unsaturated. Aryl includes, but is not limited to, phenyl, indanyl, 1-naphthyl, 2-naphthyl, tetrahydronaphthyl, 1-anthracenyl, 2-anthracenyl, 9-anthracenyl, 1-phenanthryl, 2-phenanthryl, 3-phenanthryl, 4-phenanthryl, and 5-phenanthryl. The aryls may be connected to another group either at a suitable position on the cycloalkyl ring or the aromatic ring. For example:

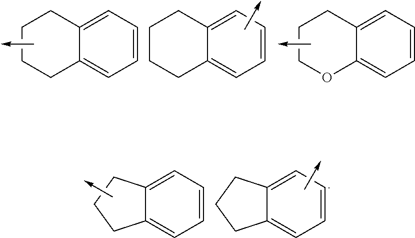

Arrowed lines drawn from the ring system indicate that the bond may be attached to any of the suitable ring atoms. For the purposes of describing the invention, the term “aryl” encompasses an “arylene.”

As used herein, the term “biphenyl” is intended to mean two phenyl groups bonded together at any one of the available sites on the phenyl ring. For example:

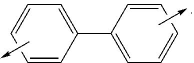

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, and fluoroscein derivatives such as:

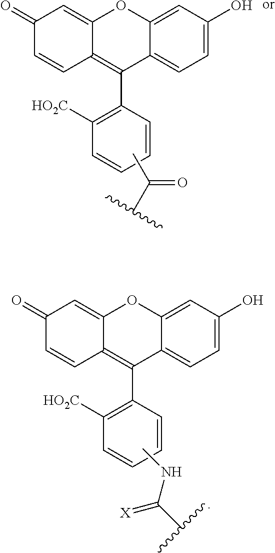

For the purposes of describing the invention, the term “heteroaryl” encompasses a “heteroarylene.”

As used herein, the term “biheteroaryl” is intended to mean a heteroaryl group substituted with another heteroaryl group at any one of the available sites on the heteroaryl ring. Biheteroaryl includes, for example:

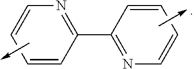

As used herein, the term “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, pyrazolinyl, and

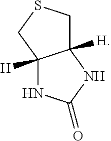

For the purposes of describing the invention, the term “heterocyclyl” encompasses a “heterocyclylene.”

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, coumarin, benzo[d][1,3]dioxole, 2,3-dihydrobenzo[b][1,4]dioxine and 3,4-dihydro-2H-benzo[b][1,4]dioxepine.

As used herein, the term “heteroatom” is intended to mean O, S or N.

As used herein, the term “activated diacid” is intended to mean a diacid wherein the carboxylic acid moieties have been transformed to, for example, but not limited to, acid halides, a succinate esters, or HOBt esters, either in situ or in a separate synthetic step. For example, succinyl chloride and terephthaloyl chloride are examples of “diacid chlorides”. HOBt esters can be formed in situ by the treatment of a diacid with a dehydrating agent such as DCC, EDC, HBTU, or others, a base such as DIPEA, and HOBt in an appropriate solvent. The reaction of an activated diacid with an amine will result in the conversion of the acid functionality to amide functionality.

As used herein, the term “detectable label” is intended to mean a group that may be linked to a compound of the present invention to produce a probe or to an IAP BIR domain, such that when the probe is associated with the BIR domain, the label allows either direct or indirect recognition of the probe so that it may be detected, measured and quantified.

As used herein, the term “affinity tag” is intended to mean a ligand or group, which is linked to either a compound of the present invention or to an IAP BIR domain to allow another compound to be extracted from a solution to which the ligand or group is attached.

As used herein, the term “probe” is intended to mean a compound of Formula 1 which is labeled with either a detectable label or an affinity tag, and which is capable of binding, either covalently or non-covalently, to an IAP BIR domain. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods of the present invention, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999), which is incorporated herein by reference in its entirety. Examples of protecting groups used throughout include, but are not limited to Fmoc, Bn, Boc, CBz and COCF3. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

As used herein, the term “subject” is intended to mean humans and non-human mammals such as primates, cats, dogs, swine, cattle, sheep, goats, horses, rabbits, rats, mice and the like.

As used herein, the term “prodrug” is intended to mean a compound that may be converted under physiological conditions or by solvolysis to a biologically active compound of the present invention. Thus, the term “prodrug” refers to a precursor of a compound of the invention that is pharmaceutically acceptable. A prodrug may be inactive or display limited activity when administered to a subject in need thereof, but is converted in vivo to an active compound of the present invention. Typically, prodrugs are transformed in vivo to yield the compound of the invention, for example, by hydrolysis in blood or other organs by enzymatic processing. The prodrug compound often offers advantages of solubility, tissue compatibility or delayed release in the subject (see, Bundgard, H., Design of Prodrugs (1985), pp. 7-9, 21-24 (Elsevier, Amsterdam). The definition of prodrug includes any covalently bonded carriers which release the active compound of the invention in vivo when such prodrug is administered to a subject. Prodrugs of a compound of the present invention may be prepared by modifying functional groups present in the compound of the invention in such a way that the modifications are cleaved, either in routine manipulation or in vivo, to a parent compound of the invention.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “BIR domain binding” is intended to mean the action of a compound of the present invention upon an IAP BIR domain, which blocks or diminishes the binding of IAPs to BIR binding proteins or is involved in displacing BIR binding proteins from an IAP. Examples of BIR binding proteins include, but are not limited to, caspases and mitochondrially derived BIR binding proteins such as Smac, Omi/WTR2A and the like.

As used herein, the term “insufficient apoptosis” is intended to mean a state wherein a disease is caused or continues because cells deleterious to the subject have not apoptosed. This includes, but is not limited to, cancer cells that survive in a subject without treatment, cancer cells that survive in a subject during or following anti-cancer treatment, or immune cells whose action is deleterious to the subject, and includes, neutrophils, monocytes, B-cells and auto-reactive T-cells.

As used herein, the term “therapeutically effective amount” is intended to mean an amount of a compound of Formula 1 which, when administered to a subject is sufficient to effect treatment for a disease-state associated with insufficient apoptosis. The amount of the compound of Formula 1 will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression or alleviation of the condition or any symptom thereof.

As used herein, the term “treating cancer” is intended to mean the administration of a pharmaceutical composition of the present invention to a subject, preferably a human, which is afflicted with cancer to cause an alleviation of the cancer (i.e., any symptom of the cancer) by killing, inhibiting the growth, or inhibiting the metastasis of the cancer cells.

As used herein, the term “preventing disease” is intended to mean, in the case of cancer, the post-surgical, post-chemotherapy or post-radiotherapy administration of a pharmaceutical composition of the present invention to a subject, preferably a human, which was afflicted with cancer to prevent the regrowth of the cancer by killing, inhibiting the growth, or inhibiting the metastasis of any remaining cancer cells. Also included in this definition is the prevention of pathogenic-cell survival in conditions that lead to diseases such as asthma, MS and the like.

As used herein, the term “synergistic effect” is intended to mean that the effect achieved with the combination of the compounds of the present invention and either the chemotherapeutic agents or death receptor agonists of the invention is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “apoptosis” or “programmed cell death” is intended to mean the regulated process of cell death wherein a dying cell displays a set of well-characterized biochemical hallmarks that include cell membrane blebbing, cell soma shrinkage, chromatin condensation, and DNA laddering, as well as any caspase-mediated cell death.

As used herein, the term “BIR domain” or “BIR” are used interchangeably throughout and are intended to mean a domain which is characterized by a number of invariant amino acid residue including conserved cysteines and one conserved hisitidine residue within the sequence Cys-(Xaa1)₂Cys-(Xaa1)₁₆His-(Xaa1)₆₋₈Cys. The BIR domain residues are listed below (see Genome Biology (2001) 1-10):

| | XIAP | HIAP-1 | HIAP-2 |
|---|---|---|---|
| BIR1 | 21-93 | 41-113 | 24-96 |
| BIR2 | 159-230 | 179-250 | 164-235 |
| BIR3 | 258-330 | 264-336 | 250-322 |
| Seq. # | P98170 | XP-006266 | XP-006267 |

As used herein, the term “ring zinc finger” or “RZF” is intended to mean a domain having the amino acid sequence of the consensus sequence: Glu-Xaa1-Xaa1-Xaa1-Xaa1-Xaa1-Xaa-1-Xaa2-Xaa1-Xaa1-Xaa1-Cys-Lys-Xaa3-Cys-Met-Xaa1-Xaa1-Xaa1-Xaa1-Xaa1-Xaa3-Xaa1-Phe-Xaa1-Pro-Cys-Gly-His-Xaa1-Xaa1-Xaa1-Cys-Xaa1-Xaa1-Cys-Ala-Xaa1-Xaa-1-Xaa1-Xaa1-Xaa1-Cys-Pro-Xaa1-Cys, wherein Xaa1 is any amino acid, Xaa2 is Glu or Asp, and Xaa3 is Val or Ile.

As used herein, the term “IAP” is intended to mean a polypeptide or protein, or fragment thereof, encoded by an IAP gene. Examples of IAPs include, but are not limited to human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apolion/BRUCE (Birc 6) (see for example U.S. Pat. Nos. 6,107,041; 6,133,437; 6,156,535; 6,541,457; 6,656,704; 6,689,562; Deveraux and Reed, Genes Dev. 13, 239-252, 1999; Kasof and Gomes, J. Biol. Chem., 276, 3238-3246, 2001; Vucic et al., Curr. Biol. 10, 1359-1366, 2000; Ashab et al. FEBS Lett., 495, 56-60, 2001, the contents of which are hereby incorporated by reference).

As used herein, the term “IAP gene” is intended to mean a gene encoding a polypeptide having at least one BIR domain and which is capable of modulating (inhibiting or enhancing) apoptosis in a cell or tissue. The IAP gene is a gene having about 50% or greater nucleotide sequence identity (preferably 95% or greater sequence identity or 100% sequence identity) to at least one of human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apolion/BRUCE (Birc 6). The region of sequence over which identity is measured is a region encoding at least one BIR domain and a ring zinc finger domain. Mammalian IAP genes include nucleotide sequences isolated from any mammalian source.

As used herein, the term “IC₅₀” is intended to mean an amount, concentration or dosage of a particular compound of the present invention that achieves a 50% inhibition of a maximal response, such as displacement of maximal fluorescent probe binding in an assay that measures such response.

As used herein, the term “EC₅₀” is intended to mean an amount, concentration or dosage of a particular compound of the present invention that achieves a 50% inhibition of cell survival.

As used herein, the term “modulate” or “modulating” is intended to mean the treatment, prevention, suppression, enhancement or induction of a function or condition using the compounds of the present invention. For example, the compounds of the present invention can modulate IAP function in a subject, thereby enhancing apoptosis by significantly reducing, or essentially eliminating the interaction of activated apoptotic proteins, such as caspase-3, 7 and 9, with the BIR domains of mammalian IAPs or by inducing the loss of XIAP protein in a cell.

As used herein, the term “enhancing apoptosis” is intended to mean increasing the number of cells that apoptose in a given cell population either in vitro or in vivo. Examples of cell populations include, but are not limited to, ovarian cancer cells, colon cancer cells, breast cancer cells, lung cancer cells, pancreatic cancer cells, or T cells and the like. It will be appreciated that the degree of apoptosis enhancement provided by an apoptosis-enhancing compound of the present invention in a given assay will vary, but that one skilled in the art can determine the statistically significant change in the level of apoptosis that identifies a compound that enhances apoptosis otherwise limited by an IAP. Preferably “enhancing apoptosis” means that the increase in the number of cells undergoing apoptosis is at least 25%, more preferably the increase is 50%, and most preferably the increase is at least one-fold. Preferably the sample monitored is a sample of cells that normally undergo insufficient apoptosis (i.e., cancer cells). Methods for detecting the changes in the level of apoptosis (i.e., enhancement or reduction) are described in the Examples and include methods that quantitate the fragmentation of DNA, methods that quantitate the translocation phosphatoylserine from the cytoplasmic to the extracellular side of the membrane, determination of activation of the caspases and methods quantitate the release of cytochrome C and the apoptosis inhibitory factor into the cytoplasm by mitochondria.

As used herein, the term “proliferative disease” or “proliferative disorder” is intended to mean a disease that is caused by or results in inappropriately high levels of cell division, inappropriately low levels of apoptosis, or both. For example, cancers and autoimmune disorders are all examples of proliferative diseases.

As used herein, the term “death receptor agonist” is intended to mean an agent capable of stimulating by direct or indirect contact the pro apoptotic response mediated by the death-receptors. For example, an agonist TRAIL receptor antibody would bind to TRAIL receptor (S) and trigger an apoptotic response. On the other hand, other agents such as interferon-α could trigger the release of endogeneous TRAIL and/or up regulate the TRAIL receptors in such a way that the cell pro-apoptotic response is amplified.

The compounds of the present invention, or their pharmaceutically acceptable salts, may contain one or more asymmetric centers, chiral axes and chiral planes. These compounds may, thus, give rise to enantiomers, diastereomers, and other stereoisomeric forms and may be defined in terms of absolute stereochemistry, such as (R)- or (S)-, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

### Utilities

The compounds of the present invention can be used for any purpose. However, compounds of Formula 1 as provided herein are believed to be especially useful as IAP BIR domain binding compounds. As such the compounds, compositions and method of the present invention include application to the cells or subjects afflicted with or having a predisposition towards developing a particular disease state, which is characterized by insufficient apoptosis. Thus, the compounds, compositions and methods of the present invention can be used to treat cellular proliferative diseases/disorders, which include, but are not limited to, i) cancer, ii) autoimmune disease, iii) inflammatory disorders, iv) proliferation induced post medical procedures, including, but not limited to, surgery, angioplasty, and the like. Accordingly, the invention provides a method of treating a proliferative disorder or other disease state characterized by insufficient apoptosis comprising administering to a subject in need thereof a therapeutically effective amount of a compound of the invention (e.g., a compound of Formula 1) or pharmaceutical composition comprising same, so as to treat the proliferative disorder or disease state characterized by insufficient apoptosis.

The compounds of the present invention may be particularly useful in the treatment of diseases in which there is a defect in the programmed cell-death or the apoptotic machinery (TRAIL, FAS, apoptosome), such as multiple sclerosis, artherosclerosis, inflammation, autoimmunity, rheumatoid arthritis (RA) and the like. Without wishing to be bound by any particular theory, it is believed that the compounds of the present invention act in combination with endogenous cell-death ligands, such as Fas, to induce apoptosis in synoviocytes (e.g., human synoviocytes). Thus, in another aspect, the invention provides a method of inducing apoptosis in a synoviocyte, especially human synoviocytes, comprising administering to the synoviocyte a compound of the invention alone or in combination, simultaneously or sequentially, with a cell-death ligand including, but not limited to, Fas. The synoviocyte can be in a tissue or a subject, for example, a tissue or subject afflicted with a disease associated with a defect in the programmed cell-death or the apoptotic machinery (TRAIL, FAS, apoptosome) of a synoviocyte, especially an autoimmune disease such as RA.

In particular, the compounds, compositions and methods of the present invention can be used for the treatment of cancer including solid tumors such as skin, breast, brain, lung, testicular carcinomas, and the like. Cancers that may be treated by the compounds, compositions and methods of the invention include, but are not limited to the following:

| Tissue | Example |
|---|---|
| Adrenal gland | neuroblastoma |
| Bone | osteogenic sarcoma (osteosarcoma), fibrosarcoma, |
| | malignant fibrous histiocytoma, chondrosarcoma, , |
| | Ewing's sarcoma malignant lymphoma (reticulum |
| | cell sarcoma), multiple myeloma, malignant giant |
| | cell tumor chordoma, osteochronfroma |
| | (osteocartilaginous exostoses), benign chondroma, |
| | chondroblastoma, chondromyxofibroma, osteoid |
| | osteoma and giant cell tumors |
| Cardiac | sarcoma (angiosarcoma, fibrosarcoma, |
| | rhabdomyosarcoma, liposarcoma), myxoma, |
| | rhabdomyoma, fibroma, lipoma and teratoma |
| Gastrointestinal | esophagus (squamous cell carcinoma, |
| | adenocarcinoma, leiomyosarcoma, lymphoma), |
| | stomach (carcinoma, lymphoma, leiomyosarcoma), |
| | pancreas (ductal adenocarcinoma, insulinoma, |
| | glucagonoma, gastrinoma, carcinoid tumors, vipoma), |
| | small bowel (adenocarcinoma, lymphoma, carcinoid |
| | tumors, Karposi's sarcoma, leiomyoma, hemangioma, |
| | lipoma, neurofibroma, fibroma), large bowel |
| | (adenocarcinoma, tubular adenoma, villous adenoma, |
| | hamartoma, leiomyoma) |
| Genitourinary | kidney (adenocarcinoma, Wilm's tumor |
| tract | [nephroblastoma], lymphoma, leukemia), bladder |
| | and urethra (squamous cell carcinoma, transitional |
| | cell carcinoma, adenocarcinoma), prostate |
| | (adenocarcinoma, sarcoma), testis (seminoma, |
| | teratoma, embryonal carcinoma, teratocarcinoma, |
| | choriocarcinoma, sarcoma, interstitial cell |
| | carcinoma, fibroma, fibroadenoma, adenomatoid |
| | tumors, lipoma) |
| Gynecological | uterus (endometrial carcinoma), cervix (cervical |
| | carcinoma, pre-tumor cervical dysplasia), ovaries |
| | (ovarian carcinoma [serous cystadenocarcinoma, |
| | mucinous cystadenocarcinoma, unclassified |
| | carcinoma], granulosa-thecal cell tumors, Sertoli- |
| | Leydig cell tumors, dysgerminoma, malignant |
| | teratoma), vulva (squamous cell carcinoma, |
| | intraepithelial carcinoma, adenocarcinoma, |
| | fibrosarcoma, melanoma), vagina (clear cell |
| | carcinoma, squamous cell carcinoma, |
| | botryoid sarcoma (embryonal rhabdomyosarcoma), |
| | fallopian tubes (carcinoma) |
| Hematologic | blood (myeloid leukemia [acute and chronic], acute |
| | lymphoblastic leukemia, chronic lymphocytic |
| | leukemia, myeloproliferative diseases, multiple |
| | myeloma, myelodysplastic syndrome), Hodgkin's |
| | disease, non-Hodgkin's lymphoma |
| | [malignant lymphoma] |
| Liver | hepatoma (hepatocellular carcinoma), |
| | cholangiocarcinoma, hepatoblastoma, |
| | angiosarcoma, hepatocellular adenoma, |
| | hemangioma |
| Lung | bronchogenic carcinoma (squamous cell, |
| | undifferentiated small cell, undifferentiated |
| | large cell, adenocarcinoma), alveolar (bronchiolar) |
| | carcinoma, bronchial adenoma, sarcoma, |
| | lymphoma, chondromatous hamartoma, |
| | mesothelioma |
| Nervous | skull (osteoma, hemangioma, granuloma, |
| system | xanthoma, osteitis deformans), meninges |
| | (meningioma, meningiosarcoma, gliomatosis), |
| | brain (astrocytoma, medulloblastoma, glioma, |
| | ependymoma, germinoma [pinealoma], |
| | glioblastoma multiform, oligodendroglioma, |
| | schwannoma, retinoblastoma, congenital |
| | tumors), spinal cord neurofibroma, meningioma, |
| | glioma, sarcoma) |
| Skin | malignant melanoma, basal cell carcinoma, |
| | squamous cell carcinoma, Karposi's sarcoma, moles |
| | dysplastic nevi, lipoma, angioma, dermatofibroma, |
| | keloids |

The compounds of the present invention, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions of the present invention can be prepared by mixing a compound of the present invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of a disease-state as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; encapsulating agents such as cyclodextrins or functionalized cyclodextrins, including, but not limited to, α, β, or δ-hydroxypropylcyclodextins hydroxypropylcyclodextins or Captisol; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains between 0.01 to 10% by weight of the compound of the present invention. Pharmaceutical compositions may be further diluted at the time of administration; for example a parenteral formulation may be further diluted with a sterile, isotonic solution for injection such as 0.9% saline, 5 wt % dextrose (D5W), Ringer's solution, or others.

The pharmaceutical composition of the present invention may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the present invention from about 0.1 to about 10% w/v (weight per unit volume).

The pharmaceutical composition of the present invention may be used for rectal administration to treat for example, colon cancer, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the present invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical composition of the present invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the present invention may be prepared by methodology well known in the pharmaceutical art. For example, a pharmaceutical composition intended to be administered by injection can be prepared by admixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound of the present invention, or a pharmaceutically acceptable salt thereof.

### Combination Therapy

The compounds of the present invention, or pharmaceutically acceptable salts thereof, may also be administered simultaneously with, prior to, or after administration of one or more additional therapeutic agents described herein. Such combination therapy may include administration of a single pharmaceutical dosage formulation which contains a compound of the present invention and one or more additional agents given below, as well as administration of the compound of the present invention in a pharmaceutical dosage formulation separate from one or more additional therapeutic agents. For example, a compound of the present invention and a chemotherapeutic agent, such as taxol (paclitaxel), taxotere, etoposide, cisplatin, vincristine, vinblastine, and the like, can be administered to the patient either together in a single oral dosage composition such as a tablet or capsule, or each agent administered in separate oral dosage formulations or via intravenous injection. Where separate dosage formulations are used, the compounds of the present invention and one or more additional agents can be administered at essentially the same time, i.e., concurrently, or at separately staggered times, i.e., sequentially; combination therapy is understood to include all these regimens. In addition, these compounds may synergize with molecules that may stimulate the death receptor apoptotic pathway through a direct or indirect manner. Accordingly, the compounds of the present invention may be used in combination with soluble TRAIL, an anti-TRAIL receptor antibody, or any agent or procedure that can cause an increase in circulating level of TRAIL, such as interferon-alpha or radiation.

Thus, the present invention also encompasses the use of the compounds of the present invention in combination with radiation therapy and/or one or more additional agents such as those described in WO 03/099211 (PCT/US03/15861), which is hereby incorporated by reference. Examples of such additional agents include, but are not limited to the following:

a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-γ agonist,
m) a PPAR-δ agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug,
s) a proteasome inhibitor such as Velcade and MG132 (7-Leu-Leu-aldehyde) (see He at al. in Oncogene (2004) 23, 2554-2558),
t) an HDAC inhibitor, such as sodium butyrate, phenyl butyrate, hydroamic acids, cyclin tetrapeptide and the like (see Rosato et al., Molecular Cancer Therapeutics 2003, 1273-1284),
u) an inhibitor of the chymotrypsin-like activity in the proteasome,
v) E3 ligase inhibitors,
w) a modulator of the immune system such as interferon-alpha and ionizing radiation (UVB) that can induce the release of cytokines, such as the interleukins, TNF, or induce release of Death receptor Ligands such as TRAIL,
x) a modulator of death receptors, includingTRAIL and TRAIL receptor agonists such as the humanized antibodies HGS-ETR1 and HGS-ETR2.

Additional combinations may also include agents which reduce the toxicity of the aforesaid agents, such as hepatic toxicity, neuronal toxicity, nephrotoxicity and the like.

### TRAIL Receptor Agonists

In one example, co-administration of one of the compounds of Formula 1 of the present invention with a death receptor agonist such as TRAIL, such as a small molecule or an antibody that mimics TRAIL may cause an advantageous synergistic effect. Moreover, the compounds of the present invention may be used in combination with any compounds that cause an increase in circulating levels of TRAIL. Agonist antibodies directed against the death receptors TRAIL-R1 and/or TRAIL-R2 can be used in combination with compounds of the invention. Exemplary agonist antibodies that may be used in combination with compounds of the invention include those described in U.S. Pat. No. 7,244,429; in U.S. Patent Application Publication Nos. 2007/0179086, 2002/0004227, 2006/0269554, 2005/0079172, 2007/0292411, 2006/0270837, 2006/0269555, 2004/0214235, and 2007/0298039; and in International Patent Publications WO2006/017961 and WO98/51793. Each of these publications is hereby incorporated by reference in its entirety. In preferred embodiments, compounds of the invention are used in combination with one or more of these TRAIL receptor agonist antibodies for the treatment of cancer and other neoplasms.

### Vinca Alkaloids and Related Compounds

Vinca alkaloids that can be used in combination with the nucleobase oligomers of the invention to treat cancer and other neoplasms include vincristine, vinblastine, vindesine, vinflunine, vinorelbine, and anhydrovinblastine.

Dolastatins are oligopeptides that primarily interfere with tubulin at the vinca alkaloid binding domain. These compounds can also be used in combination with the compounds of the invention to treat cancer and other neoplasms. Dolastatins include dolastatin-10 (NCS 376128), dolastatin-15, ILX651, TZT-1027, symplostatin 1, symplostatin 3, and LU103793 (cemadotin).

Cryptophycins (e.g., cryptophycin 1 and cryptophycin 52 (LY355703)) bind tubulin within the vinca alkaloid-binding domain and induce G2/M arrest and apoptosis. Any of these compounds can be used in combination with the compounds of the invention to treat cancer and other neoplasms.

Other microtubule disrupting compounds that can be used in conjunction with the compounds of the invention to treat cancer and other neoplasms are described in U.S. Pat. Nos. 6,458,765; 6,433,187; 6,323,315; 6,258,841; 6,143,721; 6,127,377; 6,103,698; 6,023,626; 5,985,837; 5,965,537; 5,955,423; 5,952,298; 5,939,527; 5,886,025; 5,831,002; 5,741,892; 5,665,860; 5,654,399; 5,635,483; 5,599,902; 5,530,097; 5,521,284; 5,504,191; 4,879,278; and 4,816,444, and U.S. patent application Publication Nos. 2003/0153505 A1; 2003/0083263 A1; and 2003/0055002 A1, each of which is hereby incorporated by reference.

### Taxanes and Other Microtubule Stabilizing Compounds

Taxanes such as paclitaxel, doxetaxel, RPR 109881A, SB-T-1213, SB-T-1250, SB-T-101187, BMS-275183, BRT 216, DJ-927, MAC-321, IDN5109, and IDN5390 can be used in combination with the compounds of the invention to treat cancer and other neoplasms. Taxane analogs (e.g., BMS-184476, BMS-188797) and functionally related non-taxanes (e.g., epothilones (e.g., epothilone A, epothilone B (EPO906), deoxyepothilone B, and epothilone B lactam (BMS-247550)), eleutherobin, discodermolide, 2-epi-discodermolide, 2-des-methyldiscodermolide, 5-hydroxymethyldiscoder-molide, 19-des-aminocarbonyldiscodermolide, 9(13)-cyclodiscodermolide, and laulimalide) can also be used in the methods and compositions of the invention.

Other microtubule stabilizing compounds that can be used in combination with the compounds of the invention to treat cancer and other neoplasms are described in U.S. Pat. Nos. 6,624,317; 6,610,736; 6,605,599; 6,589,968; 6,583,290; 6,576,658; 6,515,017; 6,531,497; 6,500,858; 6,498,257; 6,495,594; 6,489,314; 6,458,976; 6,441,186; 6,441,025; 6,414,015; 6,387,927; 6,380,395; 6,380,394; 6,362,217; 6,359,140; 6,306,893; 6,302,838; 6,300,355; 6,291,690; 6,291,684; 6,268,381; 6,262,107; 6,262,094; 6,147,234; 6,136,808; 6,127,406; 6,100,411; 6,096,909; 6,025,385; 6,011,056; 5,965,718; 5,955,489; 5,919,815; 5,912,263; 5,840,750; 5,821,263; 5,767,297; 5,728,725; 5,721,268; 5,719,177; 5,714,513; 5,587,489; 5,473,057; 5,407,674; 5,250,722; 5,010,099; and 4,939,168; and U.S. patent application Publication Nos. 2003/0186965 A1; 2003/0176710 A1; 2003/0176473 A1; 2003/0144523 A1; 2003/0134883 A1; 2003/0087888 A1; 2003/0060623 A1; 2003/0045711 A1; 2003/0023082 A1; 2002/0198256 A1; 2002/0193361 A1; 2002/0188014 A1; 2002/0165257 A1; 2002/0156110 A1; 2002/0128471 A1; 2002/0045609 A1; 2002/0022651 A1; 2002/0016356 A1; 2002/0002292 A1, each of which is hereby incorporated by reference.

Other chemotherapeutic agents that may be administered with a compound of the present invention are listed in the following Table:

| | | |
|---|---|---|
| Alkylating | cyclophosphamide | mechlorethamine |
| agents | lomustine | thiotepa |
| | busulfan | streptozocin |
| | procarbazine | chlorambucil |
| | ifosfamide | temozolomide |
| | altretamine | dacarbazine |
| | melphalan | semustine |
| | estramustine phosphate | carmustine |
| | hexamethylmelamine | |
| Platinum | cisplatin | tetraplatin |
| agents | carboplatinum | BBR-3464 (Hoffmann-La Roche) |
| | oxaliplatin | Ormiplatin |
| | ZD-0473 (AnorMED) | SM-11355 (Sumitomo) |
| | spiroplatinum | iproplatin |
| | lobaplatin (Aeterna) | AP-5280 (Access) |
| | carboxyphthalatoplatinum | |
| | satraplatin (Johnson Matthey) | |
| Antimetabolites | azacytidine | 6-mercaptopurine |
| | tomudex | hydroxyurea |
| | gemcitabine | 6-thioguanine |
| | trimetrexate | decitabine (SuperGen) |
| | capecitabine | cytarabin |
| | deoxycoformycin | clofarabine (Bioenvision) |
| | 5-fluorouracil | 2-fluorodeoxy |
| | fludarabine | cytidine |
| | floxuridine | irofulven (MGI Pharma) |
| | pentostatin | methotrexate |
| | 2-chlorodeoxyadenosine | DMDC (Hoffmann-La Roche) |
| | raltitrexed | idatrexate |
| | | ethynylcytidine (Taiho) |
| Topoisomerase | amsacrine | TAS-103 (Taiho) |
| inhibitors | rubitecan (SuperGen) | Topotecan |
| | epirubicin | elsamitrucin (Spectrum) |
| | exatecan mesylate (Daiichi) | dexrazoxanet (TopoTarget) |
| | etoposide | J-107088 (Merck & Co) |
| | quinamed (ChemGenex) | pixantrone (Novuspharma) |
| | teniposide or mitoxantrone | BNP-1350 (BioNumerik) |
| | gimatecan (Sigma-Tau) | rebeccamycin analogue (Exelixis) |
| | irinotecan (CPT-11) | CKD-602 (Chong Kun Dang) |
| | diflomotecan (Beaufour-Ipsen) | BBR-3576 (Novuspharma) |
| | 7-ethyl-10-hydroxy-camptothecin | KW-2170 (Kyowa Hakko) |
| Antitumor | dactinomycin (actinomycin D) | bleomycinic acid |
| antibiotics | amonafide | idarubicin |
| | doxorubicin (adriamycin) | bleomycin A |
| | azonafide | rubidazone |
| | deoxyrubicin | bleomycin B |
| | anthrapyrazole | plicamycinp |
| | valrubicin | mitomycin C |
| | oxantrazole | porfiromycin |
| | daunorubicin (daunomycin) | MEN-10755 (Menarini) |
| | losoxantrone | cyanomorpholinodoxorubicin |
| | epirubicin | GPX-100 (Gem Pharmaceuticals) |
| | bleomycin sulfate (blenoxane) | mitoxantrone (novantrone) |
| | therarubicin | |
| Antimitotic | paclitaxel | RPR 109881A (Aventis) |
| agents | SB 408075 (GlaxoSmithKline) | ZD 6126 (AstraZeneca) |
| | docetaxel | TXD 258 (Aventis) |
| | E7010 (Abbott) | PEG-paclitaxel (Enzon) |
| | Colchicines | epothilone B (Novartis) |
| | PG-TXL (Cell Therapeutics) | AZ10992 (Asahi) |
| | vinblastine | T 900607 (Tularik) |
| | IDN 5109 (Bayer) | IDN-5109 (Indena) |
| | Vincristine | T 138067 (Tularik) |
| | A 105972 (Abbott) | AVLB (Prescient NeuroPharma) |
| | Vinorelbine | cryptophycin 52 (Eli Lilly) |
| | A 204197 (Abbott) | azaepothilone B (BMS) |
| | Vindesine | vinflunine (Fabre) |
| | LU 223651 (BASF) | BNP-7787 (BioNumerik) |
| | dolastatin 10 (NCI) | auristatin PE (Teikoku Hormone) |
| | D 24851 (ASTAMedica) | CA-4 prodrug (OXiGENE) |
| | rhizoxin (Fujisawa) | BMS 247550 (BMS) |
| | ER-86526 (Eisai) | dolastatin-10 (NIH) |
| | mivobulin (Warner-Lambert) | BMS 184476(BMS) |
| | combretastatin A4 (BMS) | CA-4 (OXiGENE) |
| | cemadotin (BASF) | BMS 188797 (BMS) |
| | isohomohalichondrin-B | taxoprexin (Protarga) |
| | (PharmaMar) | |
| Aromatase | Aminoglutethimide | anastrazole |
| inhibitors | Exemestane | YM-511 (Yamanouchi) |
| | Letrozole | formestane |
| | atamestane (BioMedicines) | |
| Thymidylate | pemetrexed (Eli Lilly) | ZD-9331 (BTG) |
| synthase | nolatrexed (Eximias) | CoFactor ™ (BioKeys) |
| inhibitors | | |
| DNA | trabectedin (PharmaMar) | albumin + 32P (Isotope Solutions) |
| antagonists | mafosfamide (Baxter International) | O6 benzyl guanine (Paligent) |
| | glufosfamide (Baxter International) | thymectacin (NewBiotics) |
| | apaziquone (Spectrum | edotreotide (Novartis) |
| | Pharmaceuticals) | |
| Farnesyl- | arglabin (NuOncology Labs) | perillyl alcohol (DOR BioPharma) |
| transferase | tipifarnib (Johnson & Johnson) | BAY-43-9006 (Bayer) |
| inhibitors | lonafarnib (Schering-Plough) | |
| Pump | CBT-1 (CBA Pharma) | tariquidar (Xenova) |
| inhibitors | zosuquidar trihydrochloride (Eli | biricodar dicitrate (Vertex) |
| | Lilly) | MS-209 (Schering AG) |
| Histone | tacedinaline (Pfizer) | depsipeptide (Fujisawa) |
| acetyltransferase | pivaloyloxymethyl butyrate | MS-275 (Schering AG) |
| inhibitors | (Titan) | |
| | SAHA (Aton Pharma) | |
| Metallo- | Neovastat (Aeterna Laboratories) | marimastat (British Biotech) BMS- |
| proteinase | CMT-3 (CollaGenex) | 275291 (Celltech) |
| inhibitors | | |
| Ribonucleoside | gallium maltolate (Titan) | triapine (Vion) |
| reductase | tezacitabine (Aventis) | didox (Molecules for Health) |
| inhibitors | | |
| TNF alpha | virulizin (Lorus Therapeutics) | CDC-394 (Celgene) |
| agonists/ | revimid (Celgene) | |
| antagonists | | |
| Endothelin A | atrasentan (Abbott) | ZD-4054 (AstraZeneca) |
| receptor | YM-598 (Yamanouchi) | |
| antagonist | | |
| Retinoic acid | fenretinide (Johnson & Johnson) | LGD-1550 (Ligand) |
| receptor | alitretinoin (Ligand) | |
| agonists | | |
| Immuno- | Interferon | norelin (Biostar) |
| modulators | dexosome therapy (Anosys) | IRX-2 (Immuno-Rx) |
| | oncophage (Antigenics) | BLP-25 (Biomira) |
| | pentrix (Australian Cancer | PEP-005 (Peplin Biotech) |
| | Technology) | MGV (Progenics) |
| | GMK (Progenics) | synchrovax vaccines (CTL |
| | ISF-154 (Tragen) | Immuno) |
| | adenocarcinoma vaccine | beta.-alethine (Dovetail) |
| | (Biomira) cancer vaccine | melanoma vaccine (CTL Immuno) |
| | (Intercell) | CLL therapy (Vasogen) |
| | CTP-37 (A VI BioPharma) | p21 RAS vaccine (GemVax) |
| Hormonal and | estrogens | bicalutamide |
| antihormonal | Prednisone | testosterone propionate; |
| agents | conjugated estrogens | fluoxymesterone |
| | methylprednisolone | flutamide |
| | ethinyl estradiol | methyltestosterone |
| | prednisolone | octreotide |
| | chlortrianisen | diethylstilbestrol |
| | aminoglutethimide | nilutamide |
| | idenestrol | megestrol |
| | leuprolide | mitotane tamoxifen |
| | hydroxyprogesterone caproate | P-04 (Novogen) |
| | goserelin | Toremofine |
| | medroxyprogesterone | 2-methoxyestradiol (EntreMed) |
| | leuporelin | dexamethasone |
| | testosterone | arzoxifene (Eli Lilly) |
| Photodynamic | talaporfin (Light Sciences) | motexafin |
| agents | Pd-bacteriopheophorbide (Yeda) | gadolinium (Pharmacyclics) |
| | Theralux (Theratechnologies) | hypericin |
| | lutetium texaphyrin | |
| | (Pharmacyclics) | |
| Tyrosine | imatinib (Novartis) | C225 (ImClone) |
| Kinase | kahalide F (PharmaMar) | ZD4190 (AstraZeneca) |
| Inhibitors | leflunomide (Sugen/Pharmacia) | rhu-Mab (Genentech) |
| | CEP-701 (Cephalon) | ZD6474 (AstraZeneca) |
| | ZD1839 (AstraZeneca) | MDX-H210 (Medarex) |
| | CEP-751 (Cephalon) | vatalanib (Novartis) |
| | erlotinib (Oncogene Science) | 2C4 (Genentech) |
| | MLN518 (Millenium) | PKI166 (Novartis) |
| | canertinib (Pfizer) | MDX-447 (Medarex) |
| | PKC412 (Novartis) | GW2016 (GlaxoSmithKline) |
| | squalamine (Genaera) | ABX-EGF (Abgenix) |
| | phenoxodiol | EKB-509 (Wyeth) |
| | SU5416 (Pharmacia) | IMC-1C11 (ImClone) |
| | trastuzumab (Genentech) | EKB-569 (Wyeth) |
| | SU6668 (Pharmacia) | CI-1033 |
| | Sorafenib | EKB-569 |
| | Herceptin | Semaxanib |
| | Cetuximab | ZD6474 |
| | ZD1839 | PTK-787 |
| | PKI 166 | INC-1C11 |

Miscellaneous agents

| | |
|---|---|
| SR-27897 (CCK A inhibitor, Sanofi- | gemtuzumab (CD33 antibody, Wyeth |
| Synthelabo) | Ayerst) |
| BCX-1777 (PNP inhibitor, BioCryst) | CCI-779 (mTOR kinase inhibitor, Wyeth) |
| tocladesine (cyclic AMP agonist, | PG2 (hematopoiesis enhancer, |
| Ribapharm) | Pharmagenesis) |
| ranpirnase (ribonuclease stimulant, Alfacell) | exisulind (PDE V inhibitor, Cell Pathways) |
| alvocidib (CDK inhibitor, Aventis) | Immunol ™ (triclosan oral rinse, Endo) |
| galarubicin (RNA synthesis inhibitor, Dong- | CP-461 (PDE V inhibitor, Cell Pathways) |
| A) | triacetyluridine (uridine prodrug, Wellstat) |
| CV-247 (COX-2 inhibitor, Ivy Medical) | AG-2037 (GART inhibitor, Pfizer) |
| tirapazamine (reducing agent, SRI | SN-4071 (sarcoma agent, Signature |
| International) | BioScience) WX-UK1 (plasminogen |
| P54 (COX-2 inhibitor, Phytopharm) | activator inhibitor, Wilex) |
| N-acetylcysteine (reducing agent, Zambon) | TransMID-107 .TM. (immunotoxin, KS |
| CapCell ™ (CYP450 stimulant, Bavarian | Biomedix) |
| Nordic) | PBI-1402 (PMN stimulant, ProMetic |
| R-flurbiprofen (NF-kappaB inhibitor, | LifeSciences) |
| Encore) | PCK-3145 (apoptosis promotor, Procyon) |
| GCS-100 (gal3 antagonist, GlycoGenesys) | bortezomib (proteasome inhibitor, |
| 3CPA (NF-kappaB inhibitor, Active Biotech) | Millennium) |
| G17DT immunogen (gastrin inhibitor, | doranidazole (apoptosis promotor, Pola) |
| Aphton) | SRL-172 (T cell stimulant, SR Pharma) |
| seocalcitol (vitamin D receptor agonist, Leo) | CHS-828 (cytotoxic agent, Leo) |
| efaproxiral (oxygenator, Allos Therapeutics) | TLK-286 (glutathione S transferase |
| 131-I-TM-601 (DNA antagonist, | inhibitor, Telik) |
| TransMolecular) | trans-retinoic acid (differentiator, NIH) |
| PI-88 (heparanase inhibitor, Progen) | PT-100 (growth factor agonist, Point |
| eflornithine (ODC inhibitor, ILEX Oncology) | Therapeutics) |
| tesmilifene (histamine antagonist, YM | MX6 (apoptosis promotor, MAXIA) |
| BioSciences) | midostaurin (PKC inhibitor, Novartis) |
| minodronic acid (osteoclast inhibitor, | apomine (apoptosis promotor, ILEX |
| Yamanouchi) | Oncology) |
| histamine (histamine H2 receptor agonist, | bryostatin-1 (PKC stimulant, GPC Biotech) |
| Maxim) | urocidin (apoptosis promotor, Bioniche) |
| indisulam (p53 stimulant, Eisai) | CDA-II (apoptosis promotor, Everlife) |
| tiazofurin (IMPDH inhibitor, Ribapharm) | Ro-31-7453 (apoptosis promotor, La |
| aplidine (PPT inhibitor, PharmaMar) | Roche) |
| cilengitide (integrin antagonist, Merck | SDX-101 (apoptosis promotor, Salmedix) |
| KGaA) | brostallicin (apoptosis promotor, |
| rituximab (CD20 antibody, Genentech) | Pharmacia) |
| SR-31747 (IL-1 antagonist, Sanofi- | ceflatonin (apoptosis promotor, |
| Synthelabo) | ChemGenex) |

Additional combinations may also include agents which reduce the toxicity of the aforesaid agents, such as hepatic toxicity, neuronal toxicity, nephrotoxicity and the like.

Additional combinations may be used in the treatment of RA such as non-steroidal anti-inflammatory drugs (NSAIDs), analgesics, corticosteroids and disease-modifying antirheumatic drugs. Further combinations may include Kineret, Actemra, Hydroxychloroquine (Plaquenil™), Sulfasalazine (Azulfidine™), Leflunomide (Arava™) Tumor Necrosis Factor Inhibitors such as etanercept (Enbrel™, adalimumab (Humira™) and infliximab (Remicade™), T-cell costimulatory blocking agents such as abatacept (Orencia™), B cell depleting agents such as rituximab (Rituxan™), Interleukin-1 (IL-1) receptor antagonist therapy such as anakinra (Kineret™), intramuscular gold and other immunomodulatory and cytotoxic agents such as azathioprine (Imuran™) cyclophosphamide and cyclosporine A (Neoral™, Sandimmune™).

Other cotherapies for the treatment of RA include Methotrexate, Campath (alemtuzumab), anti-RANKL MAb (denosumab), anti-Blys MAb LymphoStat-B™ (belimumab), Cimzia (certolizumab pegol), p38 inhibitors, JAK inhibitors, anti-TNF agents, anti-CD20 MAbs, anti-IL/ILR targeting agents such as those which target IL-1, IL-5, IL-6 (toclizumab), 11-4, IL-13, and IL-23.

Additional combinations may be used in the treatment of MS such as Remicade™, Enbrel™, Humaira™, Kineret™, Orencia™, Rituxan™ and TYSABRI™ (natalizumab).

### Screening Assays

The compounds of the present invention may also be used in a method to screen for other compounds that bind to an IAP BIR domain. Generally speaking, to use the compounds of the invention in a method of identifying compounds that bind to an IAP BIR domain, the IAP is bound to a support, and a compound of the invention is added to the assay. Alternatively, the compound of the invention may be bound to the support and the IAP is added.

There are a number of ways in which to determine the binding of a compound of the present invention to the BIR domain. In one way, the compound of the invention, for example, may be fluorescently or radioactively labeled and binding determined directly. For example, this may be done by attaching the IAP to a solid support, adding a detectably labeled compound of the invention, washing off excess reagent, and determining whether the amount of the detectable label is that present on the solid support. Numerous blocking and washing steps may be used, which are known to those skilled in the art.

In some cases, only one of the components is labeled. For example, specific residues in the BIR domain may be labeled. Alternatively, more than one component may be labeled with different labels; for example, using I¹²⁵ for the BIR domain, and a fluorescent label for the probe.

The compounds of the invention may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the IAP biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an IAP BIR domain and a probe to form a probe:BIR domain complex in a first sample followed by adding a test compound from a second sample. The binding of the test is determined, and a change or difference in binding between the two samples indicates the presence of a test compound capable of binding to the BIR domain and potentially modulating the IAP's activity.

In one case, the binding of the test compound is determined through the use of competitive binding assays. In this embodiment, the probe is labeled with a fluorescent label. Under certain circumstances, there may be competitive binding between the test compound and the probe. Test compounds which display the probe, resulting in a change in fluorescence as compared to control, are considered to bind to the BIR region.

In one case, the test compound may be labeled. Either the test compound, or a compound of the present invention, or both, is added first to the IAP BIR domain for a time sufficient to allow binding to form a complex.

Formation of the probe:BIR domain complex typically require Incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the BIR domain.

In one case, the probe is added first, followed by the test compound. Displacement of the probe is an indication the test compound is binding to the BIR domain and thus is capable of binding to, and potentially modulating, the activity of IAP. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the BIR domain with a higher affinity. Thus, if the probe is detected on the support, coupled with a lack of test compound binding, may indicate the test compound is capable of binding to the BIR domain.

Modulation is tested by screening for a test compound's ability to modulate the activity of IAP and includes combining a test compound with an IAP BIR domain, as described above, and determining an alteration in the biological activity of the IAP. Therefore in this case, the test compound should both bind to the BIR domain (although this may not be necessary), and alter its biological activity as defined herein.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays in this invention include a fluorescent label such as Fluorescein, Oregon green, dansyl, rhodamine, tetramethyl rhodamine, texas red, Eu³⁺; a chemiluminescent label such as luciferase; colorimetric labels; enzymatic markers; or radioisotopes such as tritium, I¹²⁵ and the like. Affinity tags, which may be useful in performing the screening assays of the present invention include be biotin, polyhistidine and the like.

### Synthesis and Methodology

General methods for the synthesis of the compounds of the present invention are shown below and are disclosed merely for the purpose of illustration and are not meant to be interpreted as limiting the processes to make the compounds by any other methods. Those skilled in the art will readily appreciate that a number of methods are available for the preparation of the compounds of the present invention.

### General Procedures

Several methods for preparing symmetrically or non-symmetrically bridged compounds represented by Formula 1 may be envisioned. General methods are illustrated in Schemes 1 and 2.

Scheme 1 illustrates general synthetic procedures for the preparation of intermediates of general formula 1-ix used for the preparation of compounds of the instant invention in which Q is a substituted thiazole.

Protected amino-proline derivative 1-i was amidated using ammonium hydrogen carbonate and then converted to the corresponding thioamide 1-iii using Lawesson's reagent. Treatment of thioamide 1-iii with an appropriated α-bromo ketone yielded intermediate 1-iv which upon deprotection of PG¹ provided intermediate 1-v. Peptide coupling of PG³(H)N(R³)CHCO₂H was effected by activation of the carboxylic acid of PG³(H)N(R³)CHCO₂H with peptide coupling agents, followed by the addition of 1-v to provide the fully protected amide 1-vi, which may be further deprotected at PG³ to provide intermediate 1-vii. Activation of the carboxylic acid of PG⁴(R¹)N(R²)CHCO₂H with peptide coupling agents, followed by the addition of 1-vii provided intermediate 1-viii, deprotection of PG² provided intermediate 1-ix.

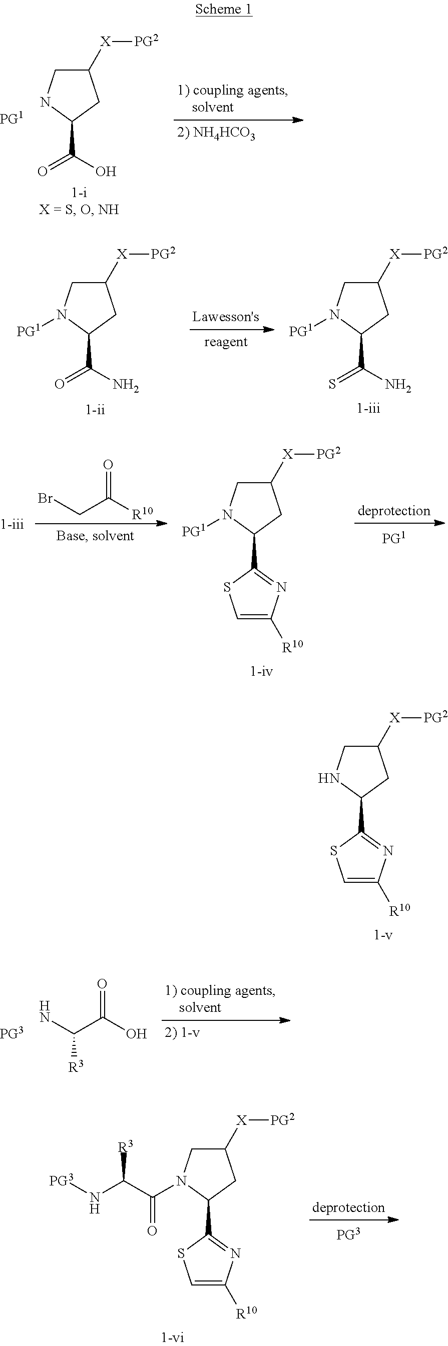

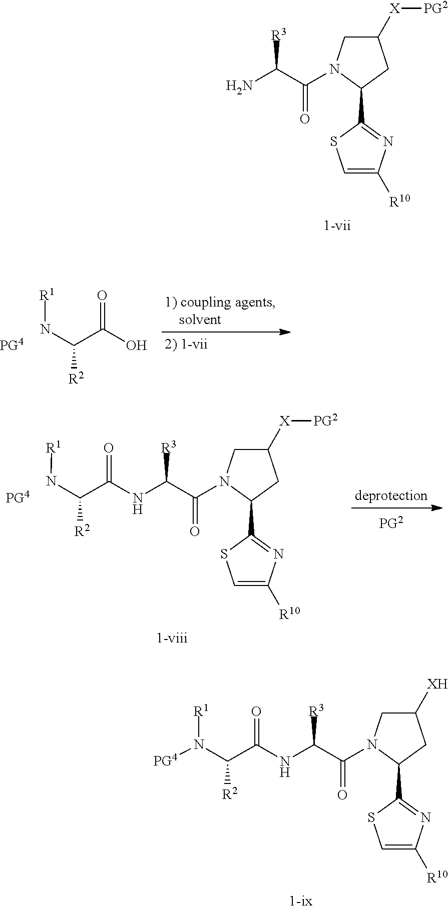

Treatment of intermediate 1-ix with LG-L-LG yielded intermediate 2-i which upon deprotection at PG⁴ provided compounds of formula 2-ii.

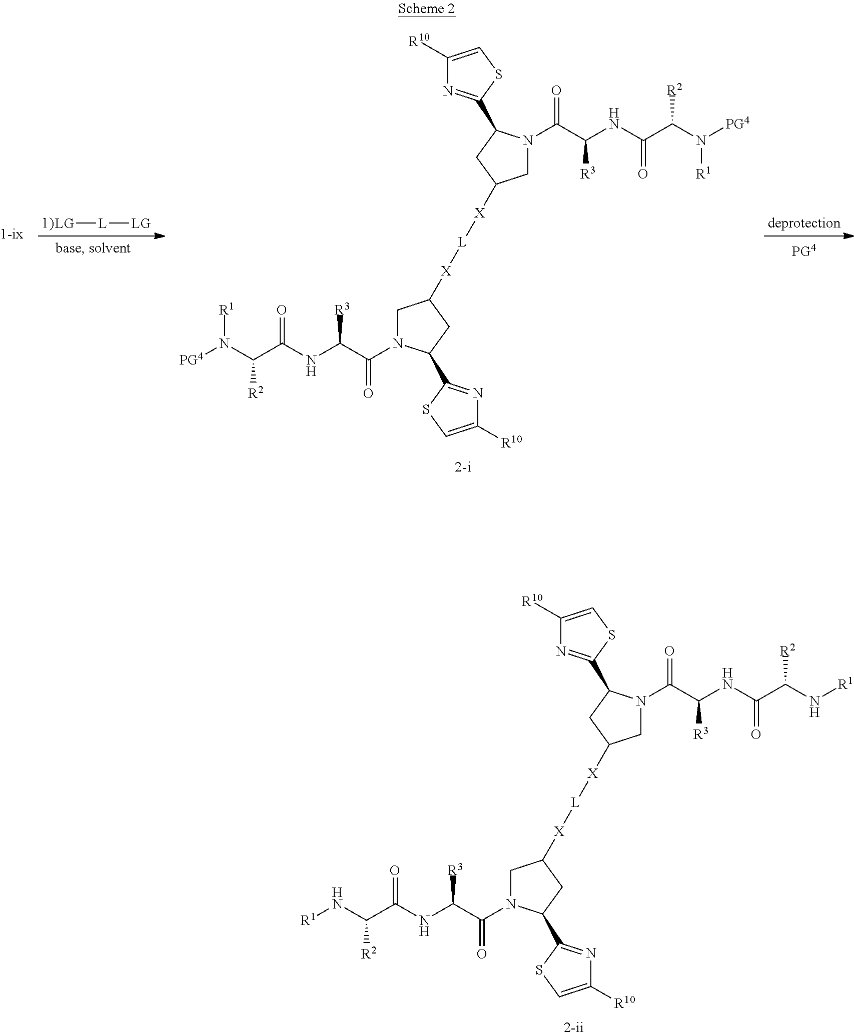

Scheme 3 illustrates general synthetic procedures for the preparation of intermediates of general formula 3-viii used for the preparation of compounds of the instant invention in which Q is a substituted oxazole.

Peptide coupling of intermediate 1-i was effected by activation of the carboxylic acid of 1-i with peptide coupling agents, followed by the addition of an appropriated amino alcohol to provide alcohol 3-i. Oxidation of alcohol 3-i provided aldehyde 3-ii which was then converted to the expected oxazole intermediate 3-iii. Deprotection of PG¹ provided intermediate 3-iv. Peptide coupling of PG³(H)N(R³)CHCO₂H is effected by activation of the carboxylic acid of PG³(H)N(R³)CHCO₂H with peptide coupling agents, followed by the addition of 3-iv to provide the fully protected amide 3-v, which may be further deprotected at PG³ to provide intermediate 3-vi. Activation of the carboxylic acid of PG⁴(R¹)N(R²)CHCO₂H with peptide coupling agents, followed by the addition of 3-vi provided intermediate 3-vii, deprotection of PG² provided intermediate 3-viii.

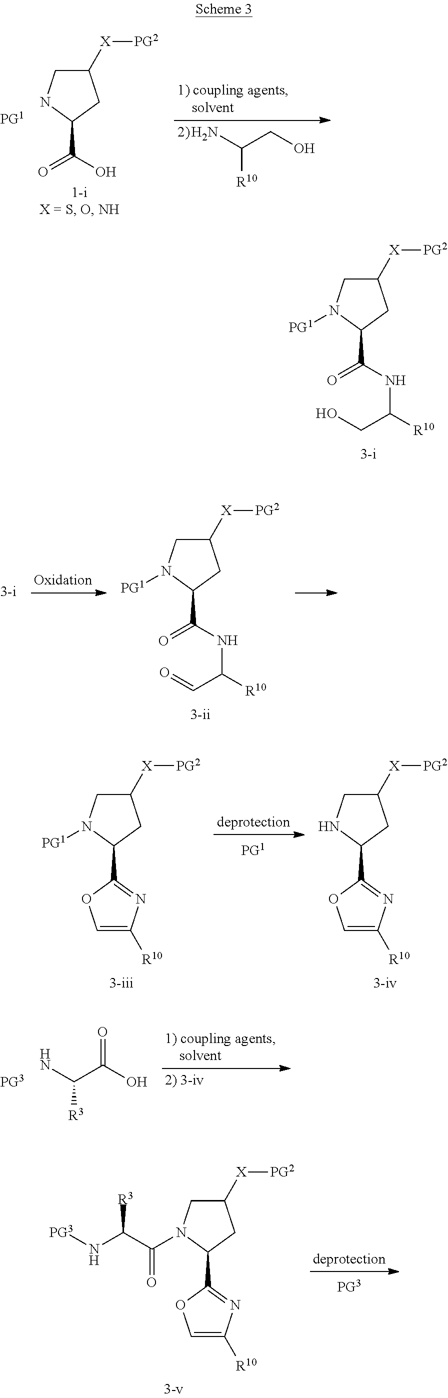

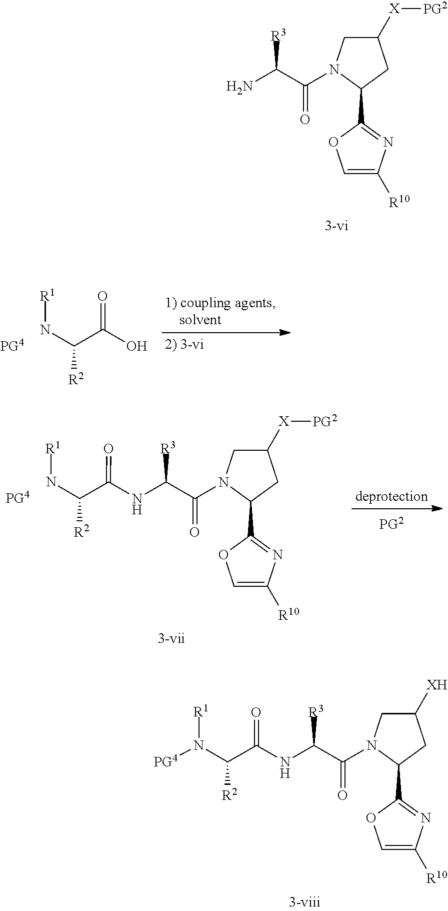

Treatment of intermediate 3-viii with LG-L-LG yielded intermediate 4-i which upon deprotection at PG⁴ provided compound of formula 4-ii.

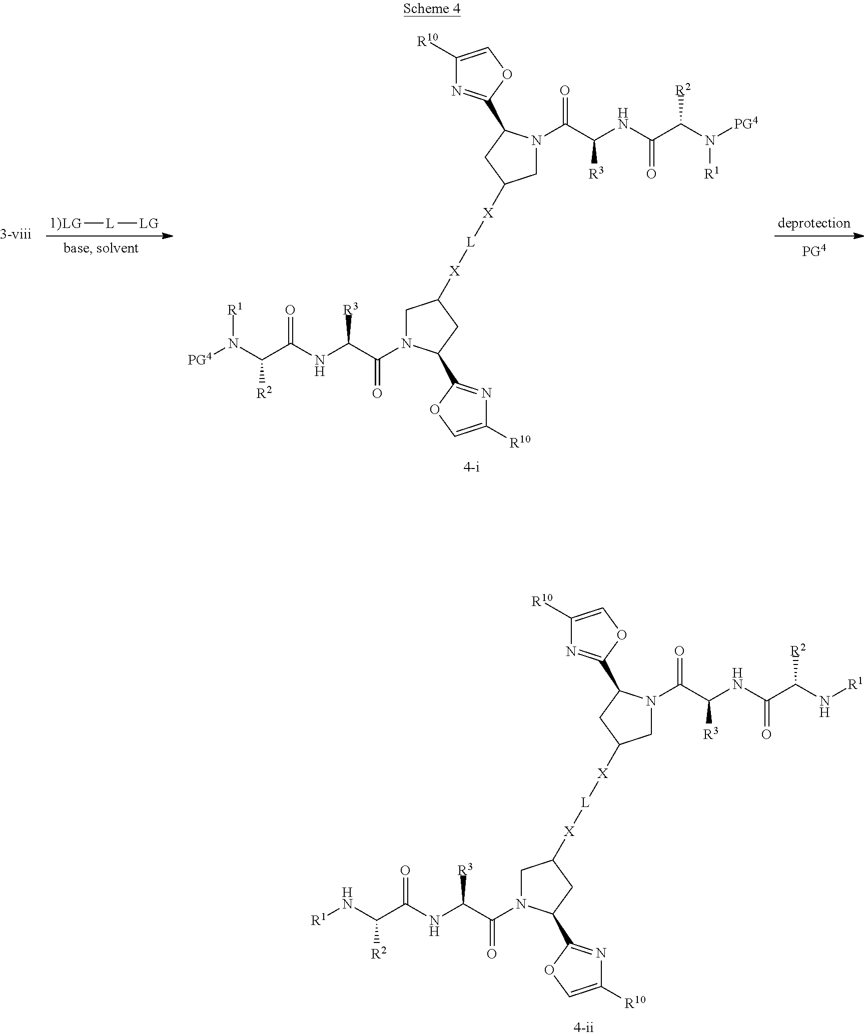

### EXAMPLES

The following abbreviations are used throughout:

Bn; benzyl;
Boc: t-butoxycarbonyl;
CBz: benzyloxycarbonyl;
DCM: dichloromethane, CH₂Cl₂;
DIPEA: diisopropylethylamine;
DMAP: 4-(dimethylamino)pyridine;

### DMF: N,N-dimethylformamide;

DTT: dithiothreitol;
EDC: 3-dimethylaminopropyl-3-ethylcarbodiimide hydrochloride;
EDTA: ethylenediaminetetracetic acid;
Fmoc: N-(9-fluorenylmethoxycarbonyl);
HBTU: O-(benzotriazol-1-yl)-N,N,N′,N′-tetramethyluronium hexafluorophosphate;
HCl: hydrochloric acid;
HOAc: acetic acid;
HOBt: 1-hydroxybenzotriazole;
HPLC: high performance liquid chromatography;
LCMS: liquid chromatography-mass spectrometer;
MeOH: methanol;
MgSO₄: magnesium sulfate;
MS: mass spectrum;
NaHCO₃: sodium hydrogen carbonate;
Pd/C: palladium on carbon;
TEA: triethylamine;
THF: tetrahydrofuran; and

### TMEDA: N,N,N,N-tetramethylethylenediamine.

LG Leaving group
PG Protective group

### Synthetic Methods

The following section summarizes synthetic methods used in the synthesis of compounds of the instant invention.

The preparation of intermediate 5-f is illustrated in scheme 5. The conversion of intermediate 5-f to compound 1 is summarize in scheme 6.

### Synthesis of Intermediate 5-i

N-Boc-cis-4-amino-Fmoc-amino-L-proline (5-a) was converted to amide 5-b using HBTU, HOBt and ammonium hydrogen carbonate. Amide 5-b was then treated with Lawesson's reagent to provide thioamide 5-c. Reaction of intermediate 5-c with 2-bromo-1-phenylethanone provided thiazole 5-d, followed by TFA deprotection yielded intermediate 5-e•TFA. Intermediate 5-e•TFA was then coupled to Boc-tBu-Gly-OH using HBTU and HOBt to provide intermediate 5-f, and TFA deprotection yielded intermediate 5-g•TFA. Intermediate 5-g•TFA was coupled to Boc-N-MeAla-OH using HBTU and HOBt to provide intermediate 5-h. Fmoc deprotection using diethylamine provided intermediate 5-i.

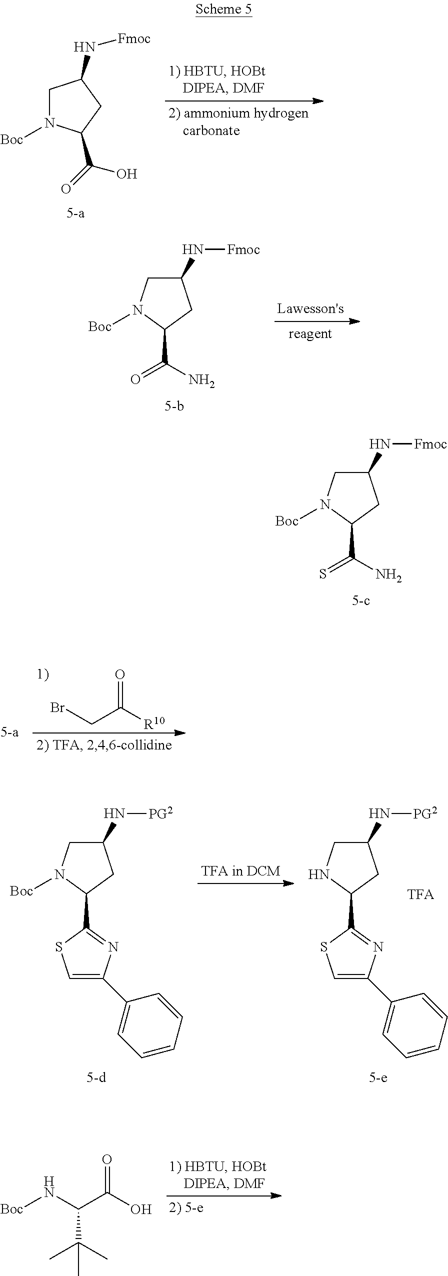

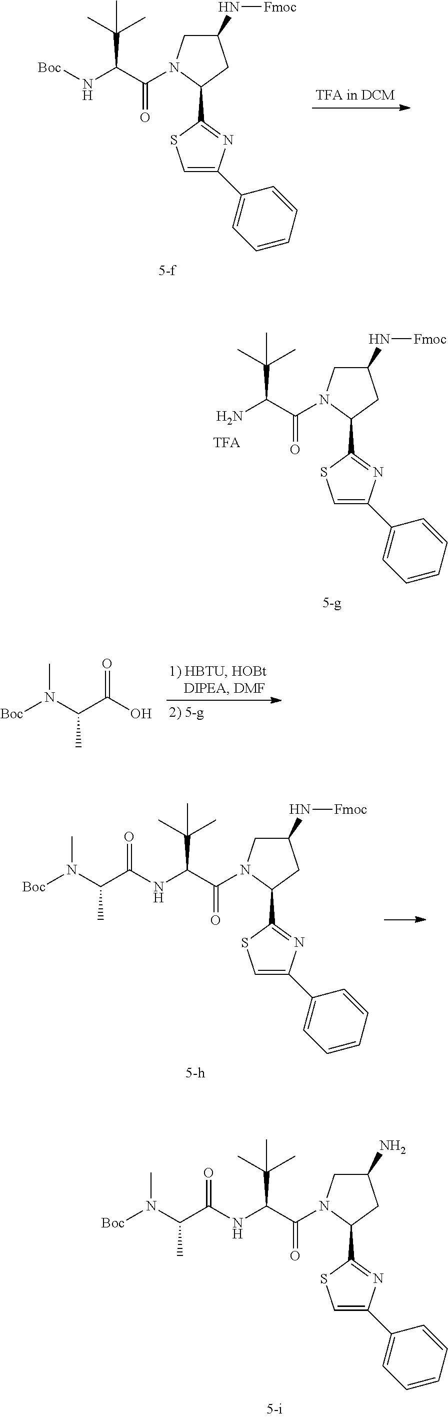

### Step 1: Intermediate 5-b

To a solution of N-Boc-cis-4-amino-Fmoc-amino-L-proline (3-a) (2.0 g, 4.42 mmol) in DMF cooled to 0° C., were sequentially added DIPEA (7.72 mL, 44.2 mmol), HOBt (776 mg, 5.75 mmol) and HBTU (2.17 g, 5.75 mmol). After stirring for 10 minutes ammonium hydrogen carbonate (1.04 g, 13.2 mmol) was added and the reaction mixture was stirred overnight at room temperature. Water and ethyl acetate were added, the organic layer was separated, washed with 10% citric acid, aqueous NaHCO₃ and brine, dried over anhydrous MgSO₄, filtered and concentrated in vacuo. Purification by silica gel chromatography provided intermediate 5-b as a white solid.

### Step 2: Intermediate 5-c

To a solution of intermediate 5-b (1.45 g, 3.21 mmol) in THF (6.4 mL) was added Lawesson's reagent (1.30 g, 3.21 mmol) and the reaction was stirred overnight at room temperature. Aqueous NaHCO₃ and ethyl acetate were added, the organic layer was separated, dried over anhydrous MgSO₄, filtered and concentrated in vacuo. The residue was triturated with diethyl ether and intermediate 5-c was isolated by filtration as a white solid.

### Step 3: Intermediate 5-d

To a solution of intermediate 5-c (1.25 g, 2.67 mmol) and sodium carbonate (1.80 g, 21.40 mmol) in DME cooled to 0° C. was added 2-bromo-1-phenylethanone (1.60 g, 8.02 mmol) and the mixture was stirred at room temperature for 30 minutes and then cooled to 0° C. A mixture of TFA (1.51 ml, 10.69 mmol) and 2,4,6-collidine (2.27 ml, 17.11 mmol) pre-mixed at 0° C. was added and the reaction was then stirred at 0° C. for 3.5 hours. Water and DCM were added, the organic layer was separated and the aqueous phase extracted twice with DCM, the combined organic layers were washed with 0.5 N HCl and brine, dried over anhydrous MgSO₄, filtered and concentrated in vacuo Purification by silica gel chromatography provided intermediate 5-d as a white solid solid. MS (m/z) M+H=568.4

Step 4: Intermediate 5-e•TFA

Intermediate 5-d (319 mg, 0.56 mmol) was dissolved in a mixture of CH₂Cl₂ (1.78 mL) and TFA (1.78 mL) at 0° C. The solution was stirred for 15 minutes at 0° C. and 2.5 hours at room temperature. Volatiles were removed under reduced pressure, the residue was re-dissolved in methanol and concentrated in vacuo to provide intermediate 5-e•TFA as a yellow solid. MS (m/z) M+H=468.4

### Step 5: Intermediate 5-f

To a solution of Boc-Tle-OH (325 mg, 1.40 mmol) in DMF cooled to 0° C. were sequentially added, DIPEA (491 μL, mmol), HOBt (247 mg, 1.82 mmol) and HBTU (693 mg, 1.82 mmol). After stirring for 10 minutes intermediate 5-e•TFA (326 mg, 0.56 mmol) was added and the reaction mixture was stirred overnight at room temperature. Water and ethyl acetate were added, the organic layer was separated, washed with 10% citric acid, saturated NaHCO₃ and brine, dried over anhydrous MgSO₄, filtered and concentrated in vacuo. Purification by silica gel chromatography provided intermediate 5-f as a white solid. MS (m/z) M+H=681.4

Step 6: Intermediate 5-g•TFA

Intermediate 5-f (263 mg, 0.38 mmol) was dissolved in a mixture of CH₂Cl₂ (1.2 mL) and TFA (1.2 mL) at 0° C. The solution was stirred for 15 minutes at 0° C. and 2.5 hours at room temperature. Volatiles were removed under reduced pressure and the residue was re-dissolved in methanol and concentrated in vacuo to provide intermediate 5-g•TFA as a yellow solid. MS (m/z) M+H=581.5

### Step 7: Intermediate 5-h

To a solution of Boc-NMe-Ala-OH (118 mg, 0.58 mmol) in DMF cooled to 0° C. were sequentially added, DIPEA (337 μl, 1.93 mmol), HOBt (102 mg, 0.75 mmol) and HBTU (285 mg, 0.75 mmol). After stirring for 10 minutes intermediate 5-g•TFA (268 mg, 0.38 mmol) was added and the reaction mixture was stirred overnight at room temperature. Water and ethyl acetate were added; the organic layer was separated, washed with 10% citric acid, saturated NaHCO₃, and brine, dried over anhydrous MgSO₄, filtered and concentrated in vacuo. Purification by silica gel chromatography provided intermediate 5-h as a white solid. MS (m/z) M+H=766.5

### Step 8: Intermediate 5-i

To a solution of intermediate 5-h (224 mg, 0.29 mmol) in THF cooled to 0° C. was added diethylamine (454 μl, 4.39 mmol) and the reaction was stirred for 7 hours at room temperature. Water and ethyl acetate were added, the organic layer was separated, washed with brine, dried over anhydrous MgSO₄, filtered and concentrated in vacuo. Purification by silica gel chromatography provided intermediate 5-i as a colorless oil. MS (m/z) M+H=544.4

### Synthesis of Compound 1

Treatment of a solution of intermediate 5-i with terephthaloyl dichloride and TEA provided intermediate 6-a. Boc deprotection of intermediate 6-a using 4N HCl in 1,4-dioxane yielded compound 1 as its bis-hydrochloride salt.

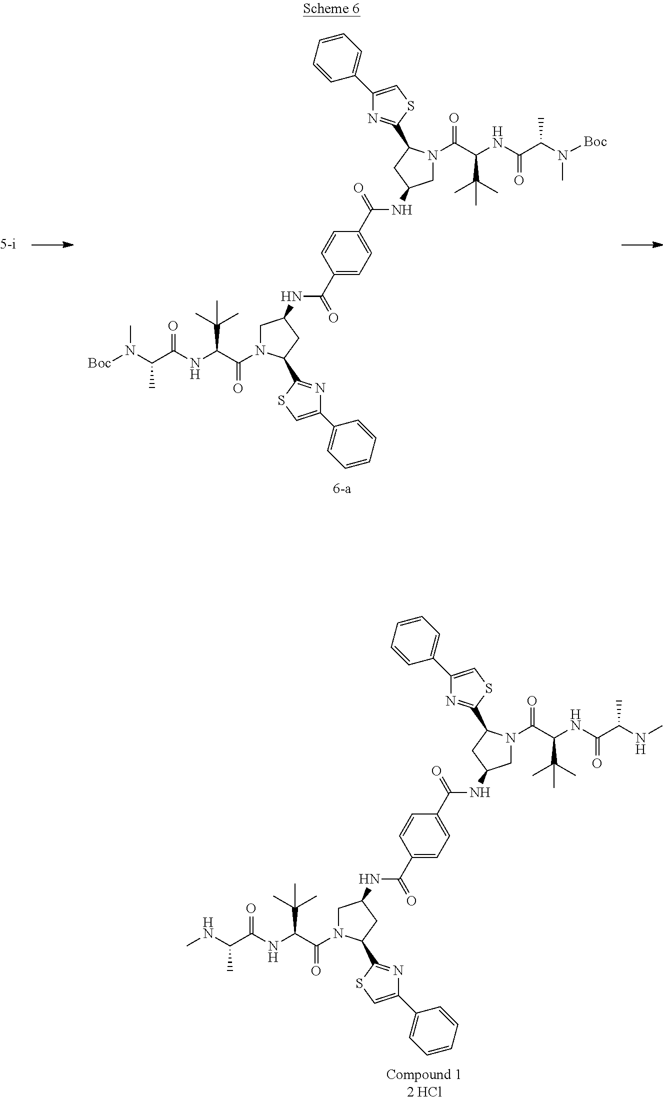

### Step 1: Intermediate 6-a

To a solution of intermediate 5-i (63 mg, 0.12 mmol) in CH₂Cl₂ cooled to 0° C. were sequentially added TEA (48.4 μl, 0.35 mmol) terephthaloyl dichloride (12.0 mg, 0.06 mmol) and DMAP (Cat) and the reaction mixture was stirred overnight at room temperature. Brine and saturated NaHCO₃ were added, the organic layer was separated, the aqueous layer was extracted twice with CH₂Cl₂, the combined organic extracts were dried over anhydrous MgSO₄, filtered and concentrated in vacuo. Purification by silica gel chromatography provided intermediate 6-a as a white solid.

### Step 2: Compound 1

4N HCl in 1,4-dioxane (787 μL) was added to intermediate 6-a (54 mg, 0.04 mmol) in MeOH (100 μL) at 0° C. and the reaction was stirred for 1.5 hour. Volatiles were removed under reduced pressure and the residue was triturated with diethyl ether to provide compound 1 as a yellow solid. MS (m/z) M+H=1017.6

### Synthesis of Intermediate 7-h

The preparation of intermediate 7-h is illustrated in scheme 7. The conversion of intermediate 7-h to compound 2 is summarized in scheme 8.

(S)-2-Amino-2-phenylethanol was coupled to N-Boc-cis-4-amino-Fmoc-amino-L-proline (5-a) using HBTU and HOBt to provide intermediate 7-a. Dess-Martin oxidation of alcohol intermediate 7-a provided the corresponding aldehyde 7-b witch was then converted to the thiazole intermediate 7-c using triphenyl phosphine and iodine, followed by TFA deprotection yielded intermediate 7-d•TFA. Intermediate 7-d•TFA was coupled to Boc-tBu-Gly-OH using HBTU and HOBt to provide intermediate 7-e, followed by TFA deprotection yielded intermediate 7-f•TFA. Intermediate 7-f•TFA was coupled to Boc-N-MeAla-OH using HBTU and HOBt to provide intermediate 7-g. Fmoc deprotection using diethylamine afforded intermediate 7-h.

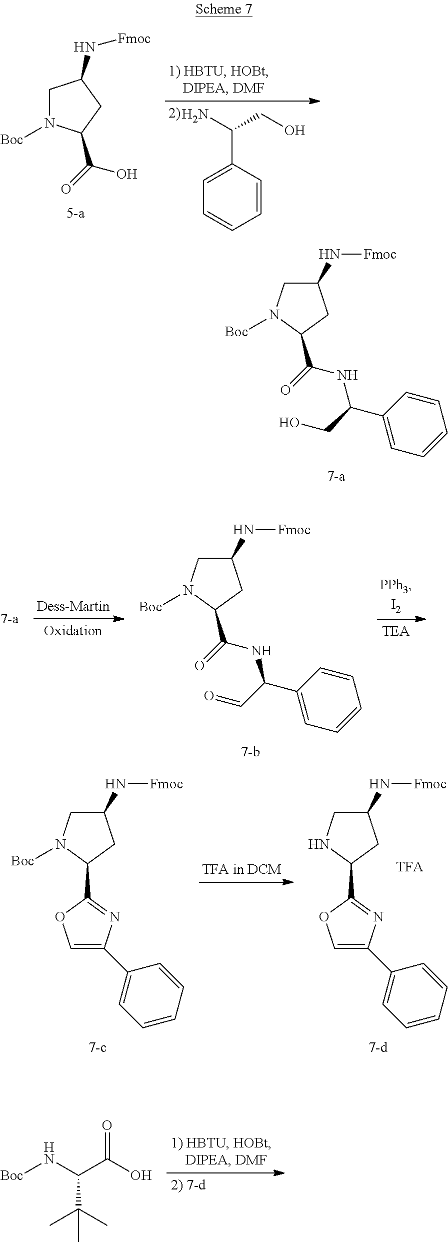

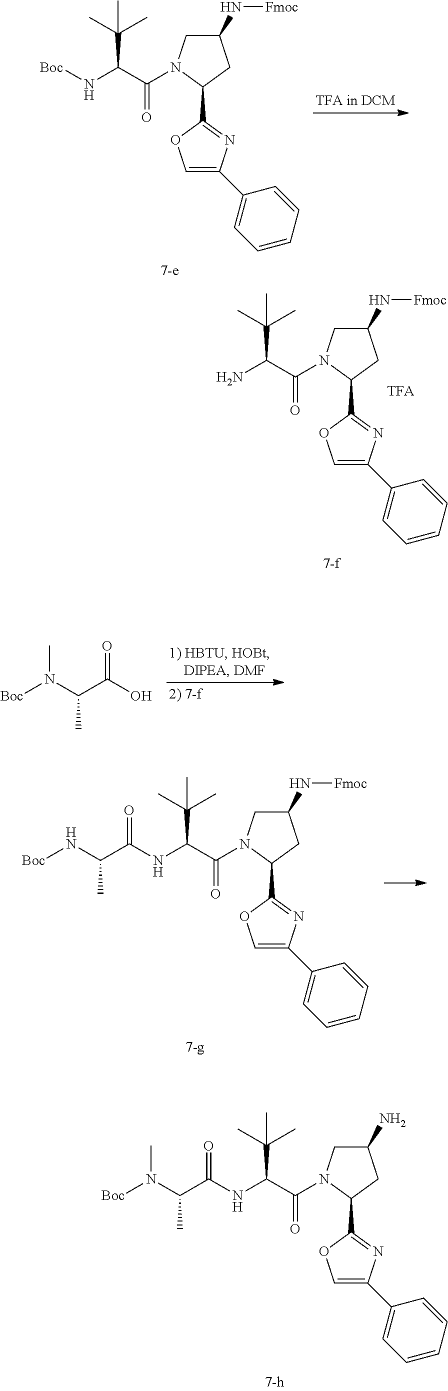

### Step 1: Intermediate 7-a

To a solution of N-Boc-cis-4-Fmoc-amino-L-proline (2.0 g, 4.42 mmol) in DMF cooled to 0° C. were sequentially added, DIPEA (3.86 mL, 22.10 mmol), HOBt (776 mg, 5.75 mmol) and HBTU (2.18 g, 5.75 mmol). After stirring for 10 minutes (S)-2-amino-2-phenylethanol (728 mg, 5.30 mmol) was added and the reaction mixture was stirred overnight at room temperature. Water and ethyl acetate were added, the organic layer was separated, washed with 10% citric acid, saturated NaHCO₃ and brine, dried over anhydrous MgSO₄, filtered and concentrated in vacuo. Purification by silica gel chromatography provided intermediate 7-a as a white solid.

### Step 2: Intermediate 7-b

To a solution of intermediate 7-a (500 mg, 0.87 mmol) in CH₂Cl₂ cooled to 0° C. was added NaHCO₃ (1.47 g, 17.50 mmol) and Dess-Martin Periodinane (891 mg, 2.10 mmol) and the reaction was stirred for 2 hours at room temperature. Water and ethyl acetate were added, the organic layer was separated, washed with aqueous Na₂S₂O₃ and brine, dried over anhydrous MgSO₄, filtered and concentrated in vacuo to provide intermediate 7-b as a yellow solid.

### Step 3: Intermediate 7-c

To a solution of triphenylphosphine (459 mg, 1.75 mmol) in CH₂Cl₂ were sequentially added iodine (222 mg, 0.87 mmol), TEA (488 μL, 3.50 mmol) and a CH₂Cl₂ solution of intermediate 7-b (498 mg, 0.87 mmol). The reaction was stirred overnight at room temperature and then concentrated in vacuo. Purification by silica gel chromatography provided intermediate 7-c as a white solid. MS (m/z) M+H=552.4

### Step 4: Intermediate 7-d

Intermediate 7-c (343 mg, 0.62 mmol) was dissolved in a mixture of CH₂Cl₂ (2.0 mL) and TFA (2.0 mL) at 0° C. The solution was stirred for 15 minutes at 0° C. and 2.5 hours at room temperature. Volatiles were removed under reduced pressure, the residue was dissolved in methanol and concentrated in vacuo to provide intermediate 7-d•TFA as a beige solid. MS (m/z) M+H=452.4

### Step 5: Intermediate 7-e

To a solution of Boc-Tle-OH (288 mg, 1.24 mmol) in DMF cooled to 0° C. were sequentially added DIPEA (543 μL, 3.11 mmol), HOBt (219 mg, 1.62 mmol) and HBTU (613 mg, 1.62 mmol). After stirring for 10 minutes intermediate 7-d•TFA (352 mg, 0.62 mmol) was added and the reaction mixture was stirred overnight at room temperature. Water and ethyl acetate were added, the organic layer was separated, washed with 10% citric acid, saturated NaHCO₃ and brine, dried over anhydrous MgSO₄, filtered and concentrated in vacuo. Purification by silica gel chromatography provided intermediate 7-e as a white solid. MS (m/z) M+H=665.5

### Step 6: Intermediate 7-f

Intermediate 7-e (355 mg, 0.53 mmol) was dissolved in a mixture of CH₂Cl₂ (1.7 mL) and TFA (1.7 mL) at 0° C. The solution was stirred for 15 minutes at 0° C. and 2.5 hours at room temperature. Volatiles were removed under reduced pressure and the residue was triturated with diethyl ether to provide intermediate 7-f•TFA as a beige solid.

### Step 6: Intermediate 7-g

To a solution of Boc-NMe-Ala-OH (163 mg, 0.80 mmol) in DMF cooled to 0° C. were sequentially added DIPEA (466 μl, 2.67 mmol), HOBt (141 mg, 1.04 mmol) and HBTU (395 mg, 1.04 mmol). After stirring for 10 minutes intermediate 7-f•TFA (302 mg, 0.53 mmol) was added and the reaction mixture was stirred overnight at room temperature. Water and ethyl acetate were added; the organic layer was separated, washed with 10% citric acid, saturated NaHCO₃, and brine, dried over anhydrous MgSO₄, filtered and concentrated in vacuo. Purification by silica gel chromatography provided intermediate 7-g as a white solid. MS (m/z) M+H=750.6

### Step 6: Intermediate 7-h

To a solution of intermediate 7-g (302 mg, 0.40 mmol) in THF cooled to 0° C. was added diethylamine (625 μl, 6.04 mmol) and the reaction was stirred for 7 hours at room temperature. Water and ethyl acetate were added, the organic layer was separated, washed with brine, dried over anhydrous MgSO₄, filtered and concentrated in vacuo. Purification by silica gel chromatography provided intermediate 7-h as a white solid.

### Synthesis of Compound 2

Treatment of a solution of intermediate 7-h with terephthaloyl dichloride and TEA provided intermediate 8-a. Boc deprotection of intermediate 8-a using 4N HCl in 1,4-dioxane yielded compound 2 as its bis-hydrochloride salt.

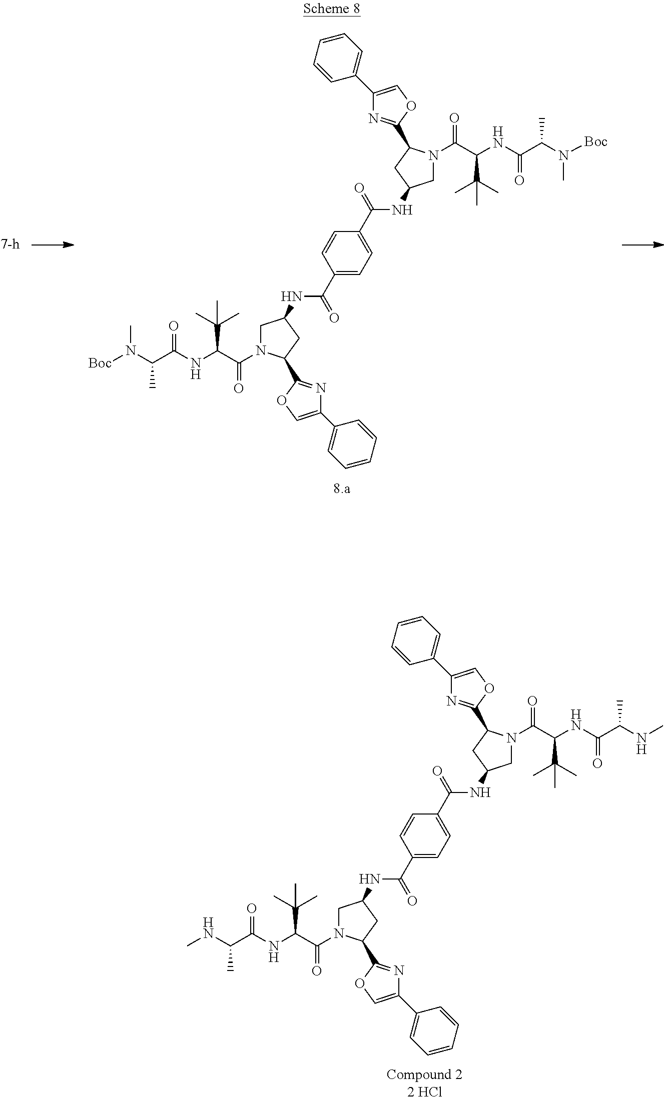

### Step 1: Intermediate 8-a

To a solution of intermediate 7-h (145 mg, 0.27 mmol) in CH₂Cl₂ cooled to 0° C. were sequentially added TEA (115 μl, 0.82 mmol), terephthaloyl dichloride (28 mg, 0.13 mmol) and DMAP (cat) and the reaction mixture was stirred overnight at room temperature. Brine and saturated NaHCO₃ were added, the organic layer was separated, the aqueous layer was extracted twice with CH₂Cl₂, the combined organic extracts were washed with brine, dried over anhydrous MgSO₄, filtered and concentrated in vacuo. Purification by silica gel chromatography provided intermediate 8-a as a white solid. MS (m/z) M+H=1185.7

### Step 2: Compound 2

4N HCl in 1,4-dioxane (1413 μL) was added to intermediate 8-a (134 mg, 0.11 mmol) in MeOH (100 μL) at 0° C. and the reaction was stirred for 1.5 hour. Volatiles were removed under reduced pressure and the residue was triturated with diethyl ether to provide compound 2.2HCl as a white solid MS (m/z) M+H=985.6

### Synthesis of Compound 13

The synthesis of compound 13 is illustrated in schemes 9 and 10.

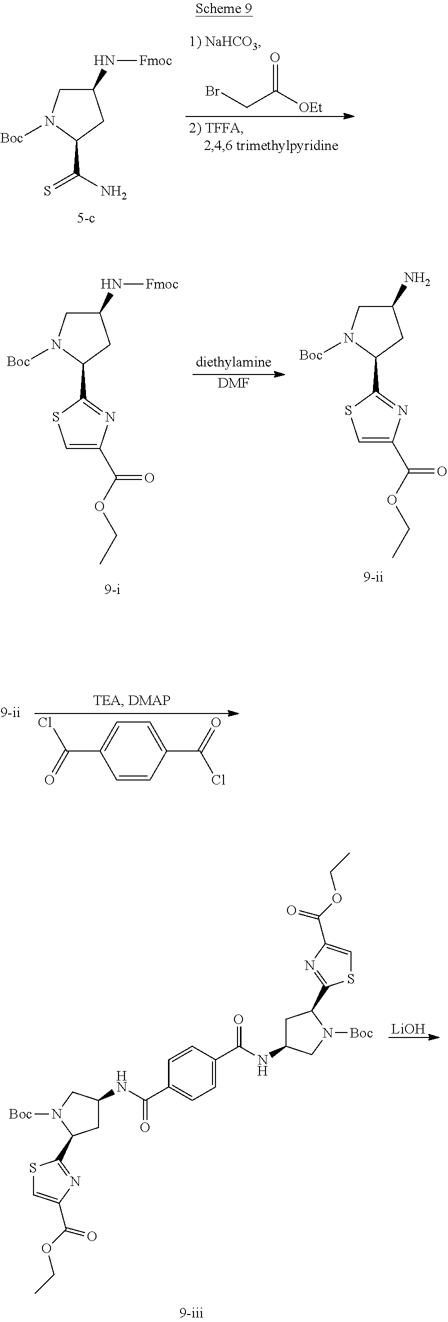

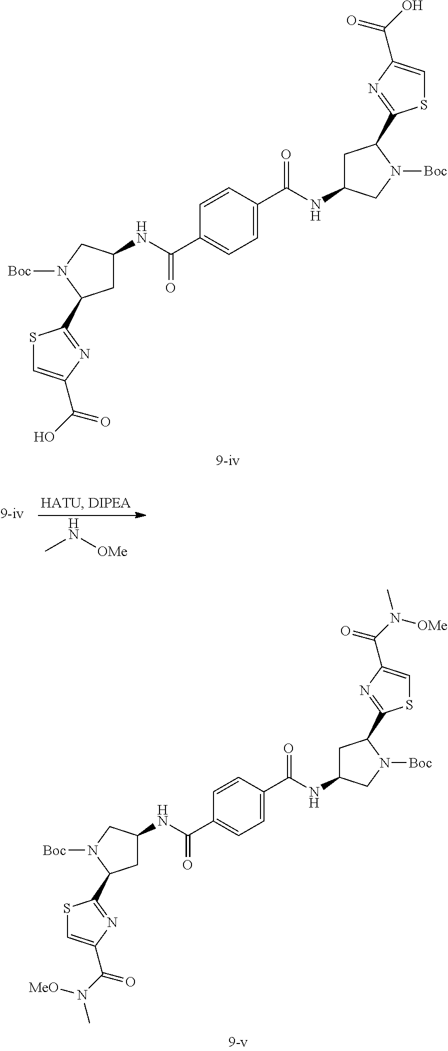

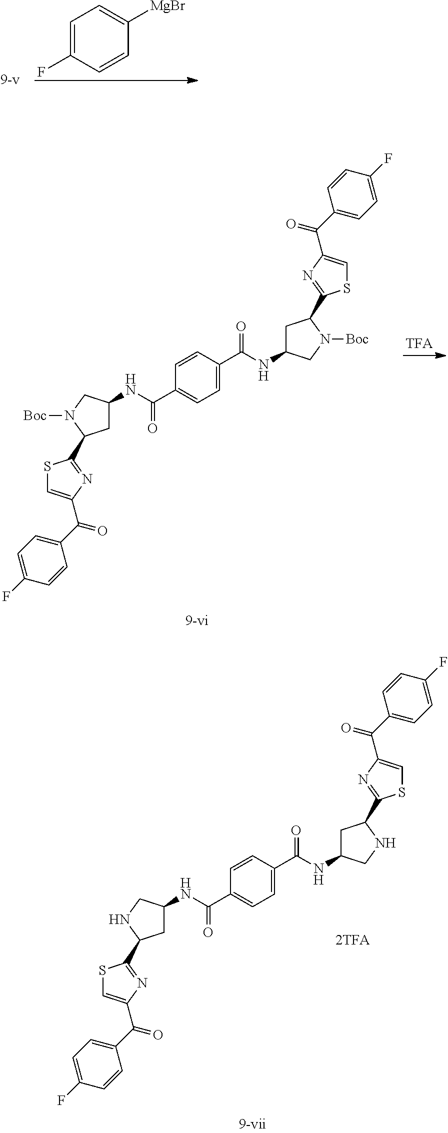

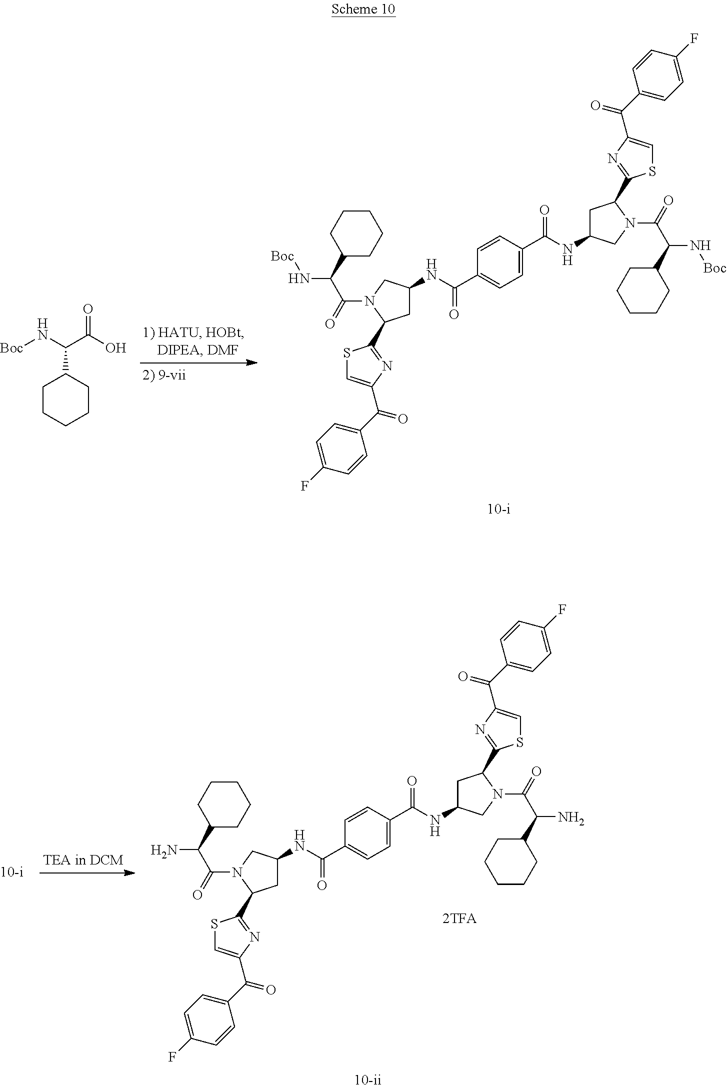

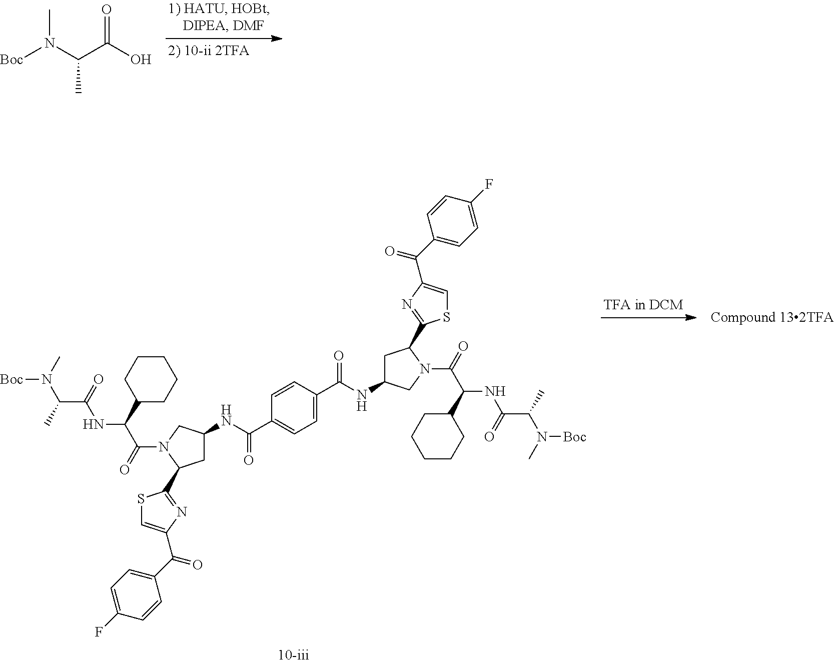

### Step 1: Intermediate 9-i

To a suspension of intermediate 5-c (2.5 g, 5.35 mmol) and NaHCO₃ (3.59 g, 42.8 mmol) in DME cooled to 0° C. was added dropwise ethyl 3-bromo-2-oxopropanoate (2.24 mL, 16.04 mmol) and the reaction was stirred for 40 minutes at room temperature and then cooled to 0° C. A solution of TFFA (4.49 g, 21.39 mmol) and 2,4,6-trimethylpyridine (5.18 g, 42.8 mmol) in DME were added and the reaction was then stirred for 2.5 hours at room temperature. Water and ethylacetate were added, the organic layer was separated, the aqueous phase was extracted with ethyl acetate, the combined organic layers were washed with 1N HCl and brine, dried over anhydrous MgSO₄, filtered and concentrated in vacuo. Purification by silica gel chromatography provided intermediate 9-i as a yellow foam. MS (m/z) M+H=564.3

### Step 2: Intermediate 9-ii

To a solution of Intermediate 9-i (1.68 g, 2.987 mmol) in THF cooled to 0° C. was added diethylamine (4.62 mL) and the reaction was stirred at room temperature for 7 hours and volatiles were removed under reduced pressure. Purification by silica gel chromatography provided intermediate 9-ii as a yellow solid. MS (m/z) M+H=342.2

### Step 3: Intermediate 9-iii

To a solution of intermediate 9-ii (535 mg, 1.56 mmol) in CH₂Cl₂ cooled to 0° C. were sequentially added TEA (655 μl, 4.70 mmol), terephthaloyl dichloride (143 mg, 0.70 mmol) and DMAP (cat) and the reaction mixture was stirred overnight at room temperature. Saturated NaHCO₃ was added, the organic layer was separated, washed with brine, dried over anhydrous MgSO₄, filtered and concentrated in vacuo. Purification by silica gel chromatography provided intermediate 9-iii as a yellow solid. MS (m/z) M+H=813.3

### Step 4: Intermediate 9-iv

To a solution of intermediate 9-iii (477 mg, 0.58 mmol) in THF cooled to 0° C. was added 2N LiOH (2.93 mL, 5.87 mmol), the reaction was stirred overnight at room temperature and then acidified to pH=2 using 1N HCl. Ethyl acetate was added the organic layer was separated, washed with brine, dried over anhydrous MgSO₄, filtered and concentrated in vacuo to provide intermediate 9-iv as a yellow solid. MS (m/z) M+H=757.3

### Step 5: Intermediate 9-v

To a solution of intermediate 9-iv (398 mg, 0.52 mmol) in DMF cooled to 0° C. were sequentially added N,O-dimethylhydroxylamine (154 mg, 1.57 mmol), HATU (435 mg, 1.15 mmol) and DIPEA (918 uL, 5.26 mmol) and the reaction was then stirred at room temperature for 2 days. Saturated ammonium chloride and ethyl acetate were added, the organic layer was separated, washed with brine, dried over anhydrous MgSO₄, filtered and concentrated in vacuo. Purification by silica gel chromatography provided intermediate 9-v as a white solid. MS (m/z) M+H=843.1

### Step 6: Intermediate 9-vi

To a solution of intermediate 9-v (243 mg, 0.28 mmol) in THF cooled to 0° C. was added dropwise (4-fluorophenyl) magnesium bromide (1.73 mL, 1.73 mmol) at −78° C. and the reaction was allowed to warm to room temperature over 2 hours. Saturated ammonium chloride and ethyl acetate were added, the organic layer was separated, washed with brine, dried over anhydrous MgSO₄, filtered and concentrated in vacuo. Purification by silica gel chromatography provided intermediate 9-vi as a white foam. MS (m/z) M+H=913.3

### Step 7: Intermediate 9-vii

To a solution of intermediate 9-vi (87 mg, 0.09 mmol) in CH₂Cl₂ (0.5 mL) cooled to 0° C. was added TFA (0.40 mL, 5.24 mmol) and the solution was then stirred for 2.5 hours at 0° C. Volatiles were removed under reduced pressure to provide intermediate 9-vii.2TFA as a white solid.

### Step 8: Intermediate 10-i

To a solution of intermediate 9-vii (74 mg, 0.09 mmol) in DMF cooled to 0° C. were sequentially added Boc-Chg-OH (73 mg, 0.28 mmol), HOAt (48 uL), HATU (108 mg, 0.28 mmol) and DIPEA (165 μL, 0.95 mmol) and the reaction mixture was stirred overnight at room temperature. Ethyl acetate and saturated ammonium chloride were added, the organic layer was separated, washed with brine, dried over anhydrous MgSO₄, filtered and concentrated in vacuo. Purification by silica gel chromatography provided intermediate 10-i as a colorless oil.

### Step 9: Intermediate 10-ii

To a solution of intermediate 10-i (87 mg, 0.07 mmol) in CH₂Cl₂ (0.5 mL) cooled to 0° C. was added TFA (0.30 mL, 4.02 mmol) and the solution was then stirred for 2.5 hours at 0° C. Volatiles were removed under reduced pressure to provide intermediate 10-ii.2TFA as a yellow foam. MS (m/z) M+H=991.2

### Step 10: Intermediate 10-iii

To a solution of intermediate 10-ii (89 mg, 0.07 mmol) in DMF cooled to 0° C. were sequentially added Boc-NMe-Ala-OH (44 mg, 0.21 mmol), HOAt (37 uL, 0.02 mmol), HATU (83 mg, 0.21 mmol) and DIPEA (127 μL, 0.73 mmol) and the reaction mixture was stirred overnight at room temperature. Ethyl acetate and saturated NaHCO₃ were added, the organic layer was separated, washed with brine, dried over anhydrous MgSO₄, filtered and concentrated in vacuo. Purification by silica gel chromatography provided intermediate 10-iii as a yellow solid.

### Step 11: Compound 13

To a solution of intermediate 10-iii (55 mg, 0.04 mmol) in CH₂Cl₂ (0.4 mL) cooled to 0° C. was added TFA (0.17 mL, 2.22 mmol) and the solution was then stirred for 2.5 hours at 0° C. Volatiles were removed under reduced pressure and the residue was triturated with diethyl ether to provide compound 13.2TFA as a yellow solid. MS (m/z) M+H=1161.4

Representative compounds of the present invention were prepared according to variations of the above procedures and are illustrated in Table 1:

TABLE 1

| COM- | | |
|---|---|---|
| POUND | STRUCTURE | MS (m/z) |
| 1 | 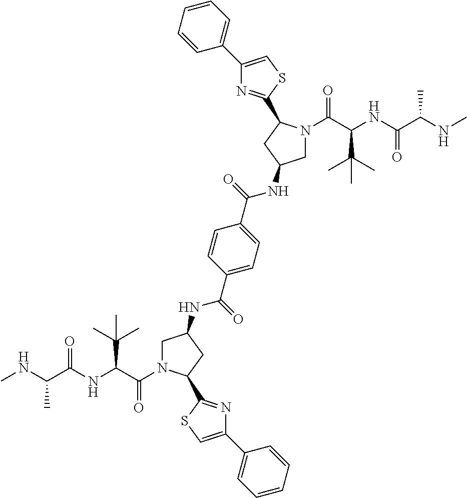 | [M + H]⁺ = 1017.6 |
| 2 | 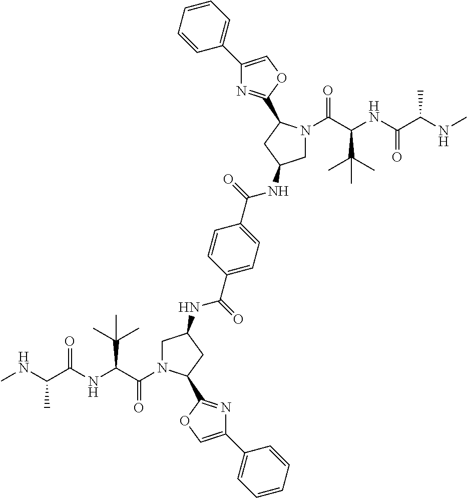 | [M + H]⁺ = 985.6 |
| 3 | 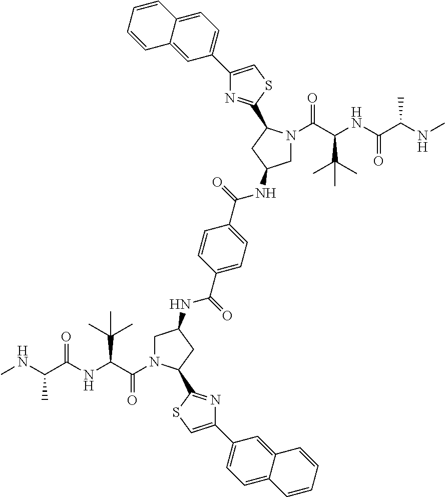 | [M + H]⁺ = 1117.7 |
| 4 | 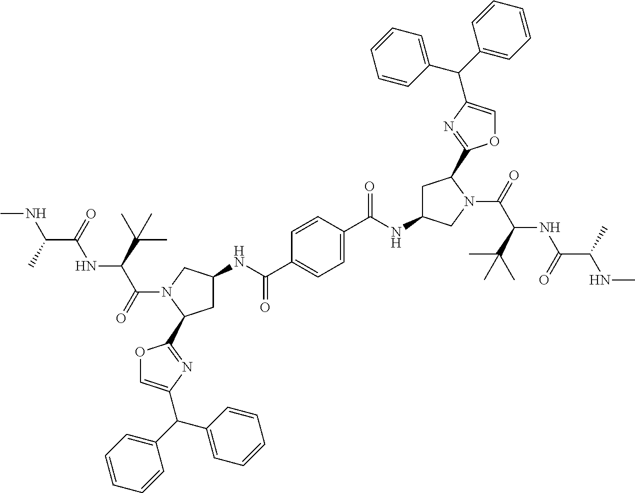 | [M + H]⁺ = 1165.8 |
| 5 | 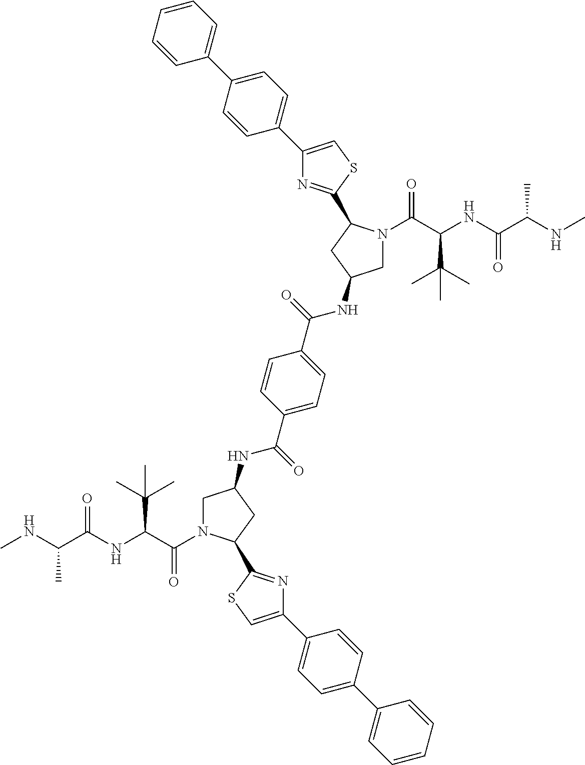 | [M + 2H]⁺ = 585.7 |
| 6 | 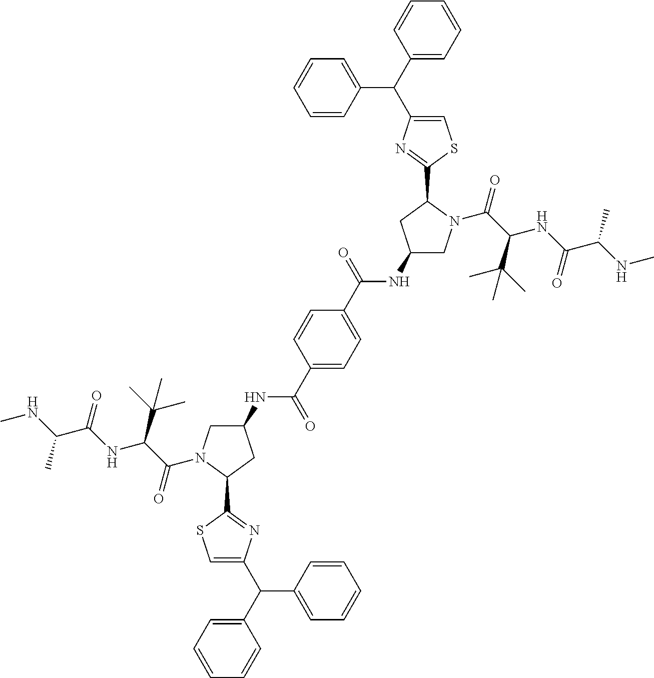 | [M + H]⁺ = 1197.7 |
| 7 | 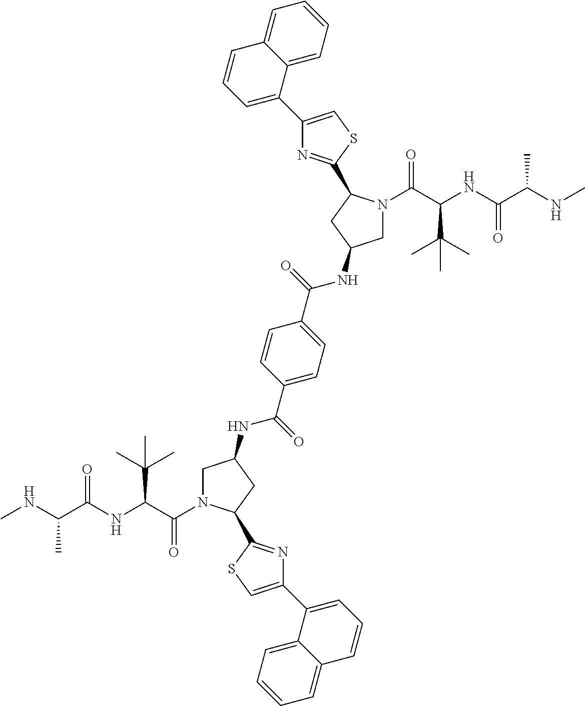 | [M + H]⁺ = 1117.5 |
| 8 | 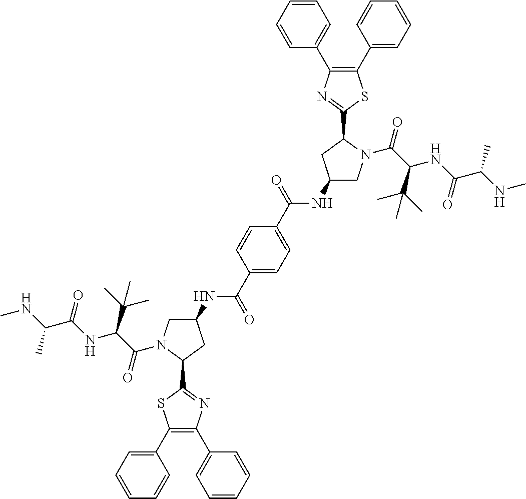 | [M + H]⁺ = 1170.4 |
| 9 | 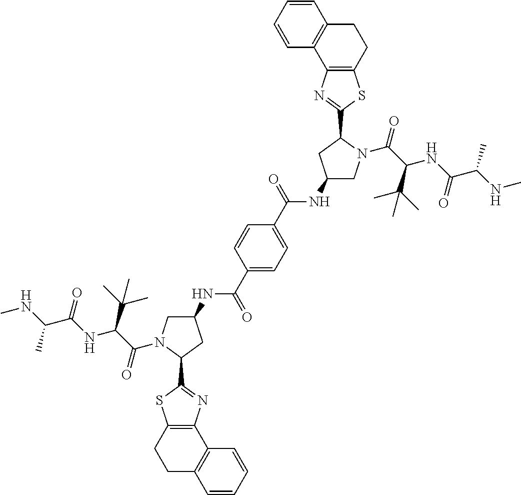 | [M + H]⁺ = 1069.5 |
| 10 | 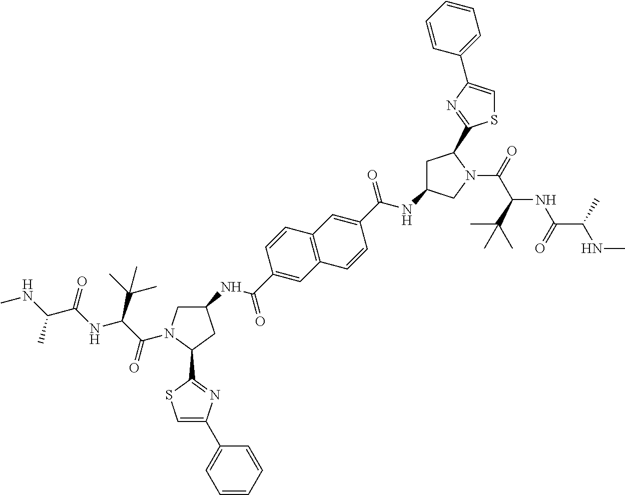 | [M + H]⁺ = 1067.3 |
| 11 | 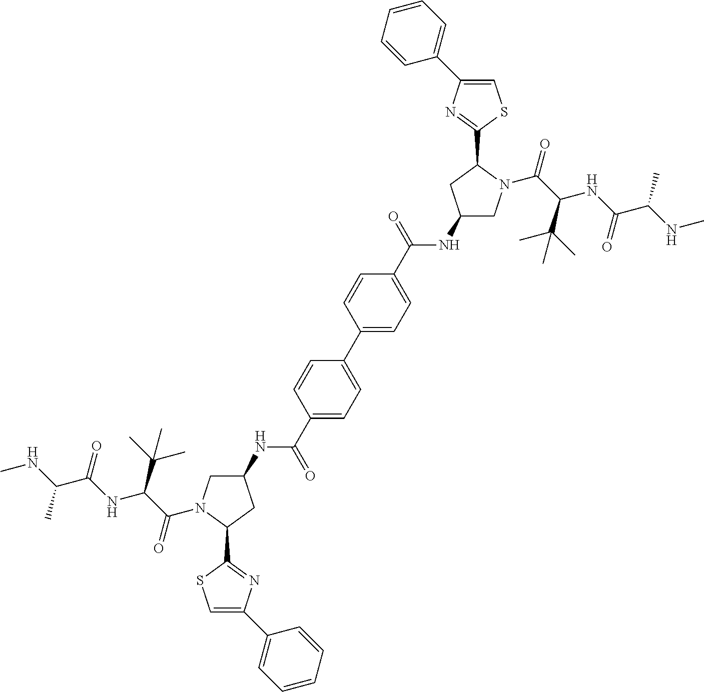 | [M + 2H]⁺ = 547.5 |
| 12 | 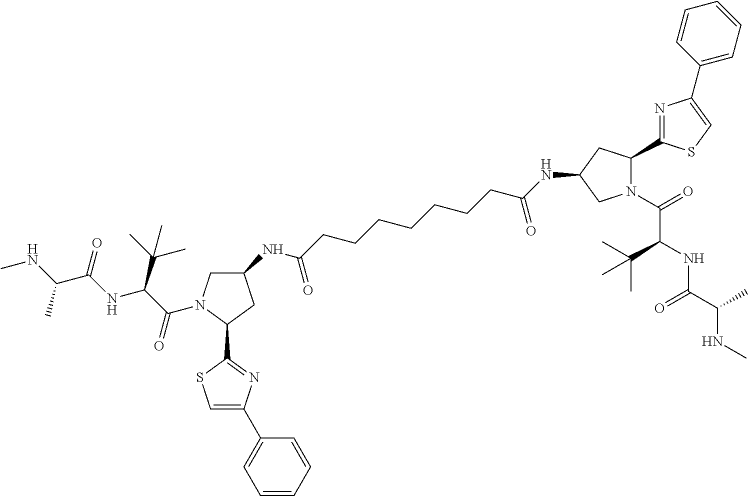 | [M + H]⁺ = 1039.5 |
| 13 | 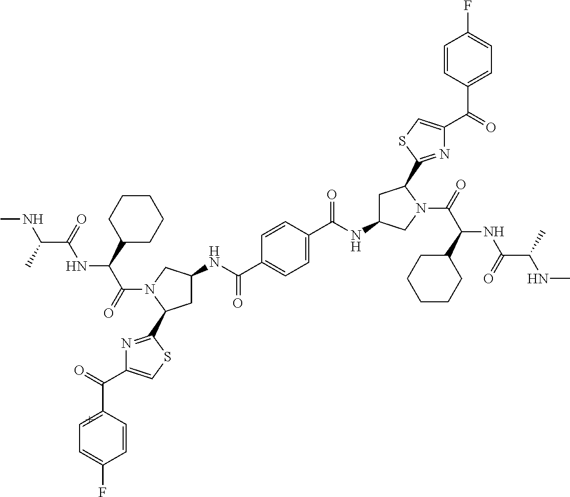 | [M + H]⁺ = 1161.4 |
| 14 | 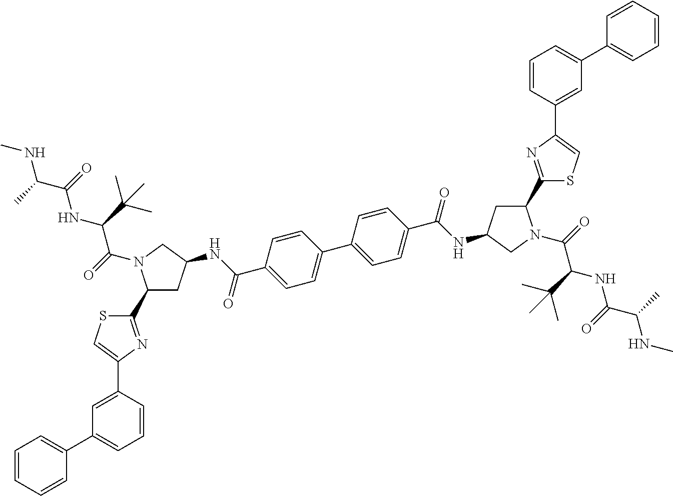 | [M + 2H]⁺ = 623.6 |
| 15 | 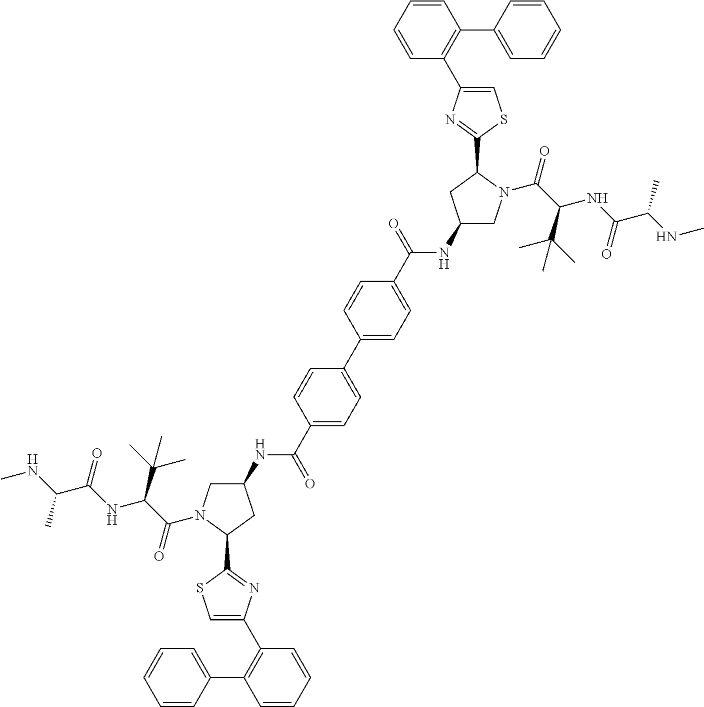 | [M + 2H]⁺ = 623.5 |
| 16 | 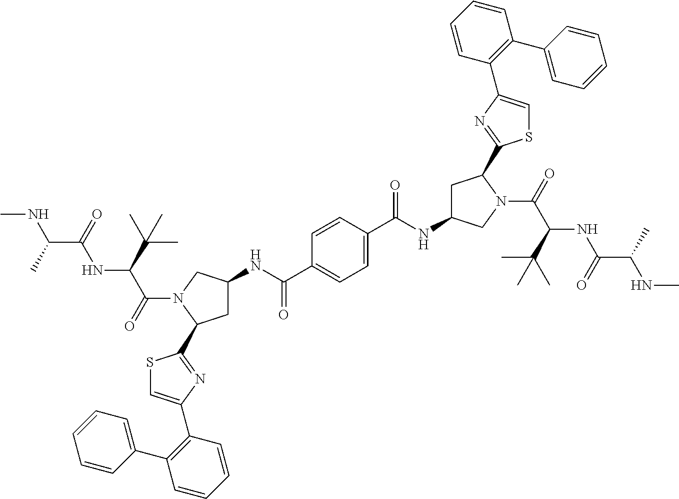 | [M + H]⁺ = 1170.4 |

Other compounds of the instant invention include those of Table 2:

TABLE 2
STRUCTURE

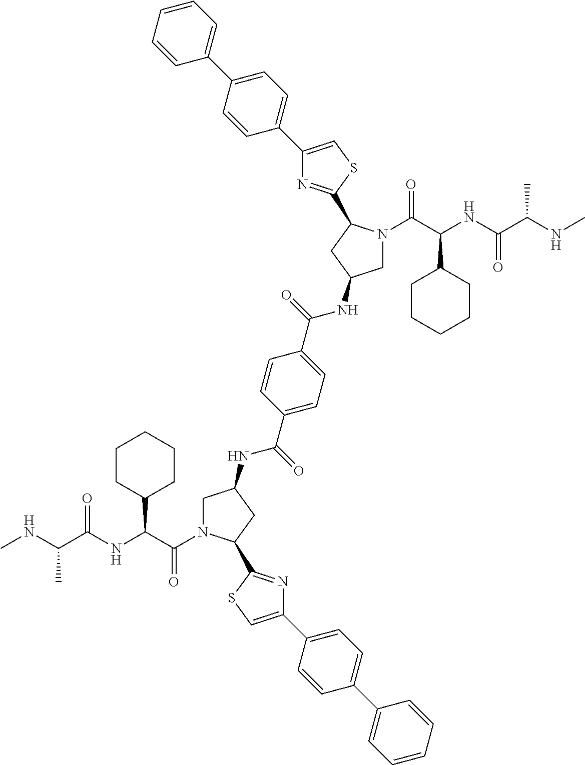

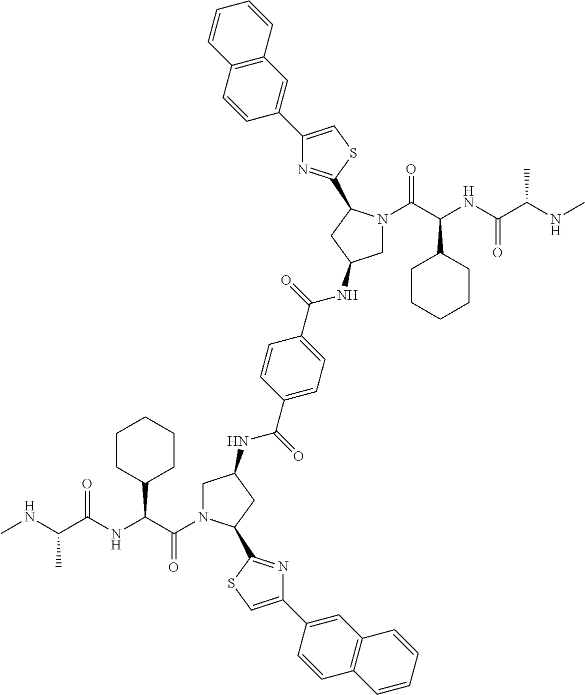

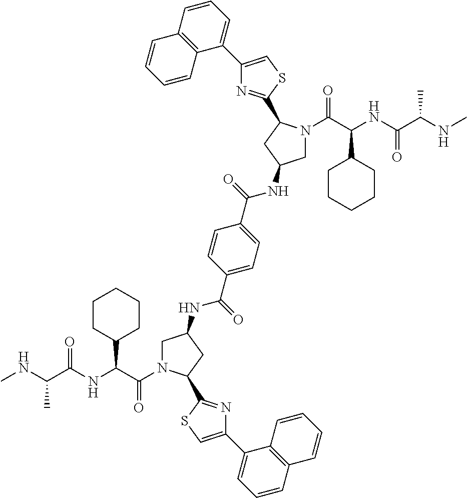

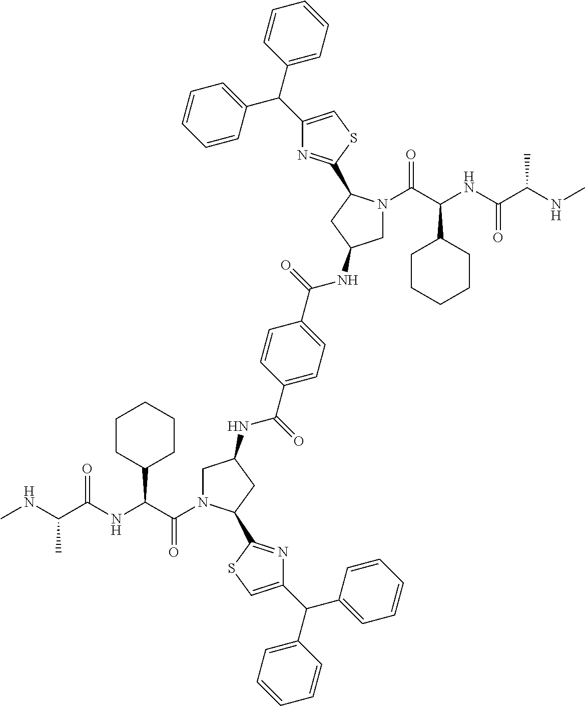

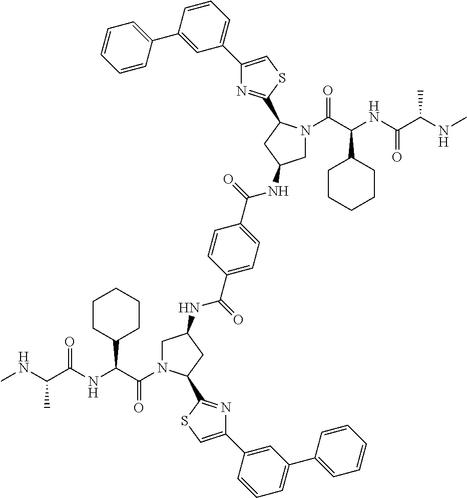

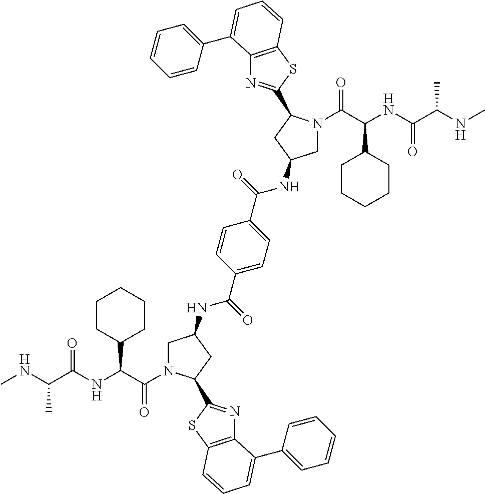

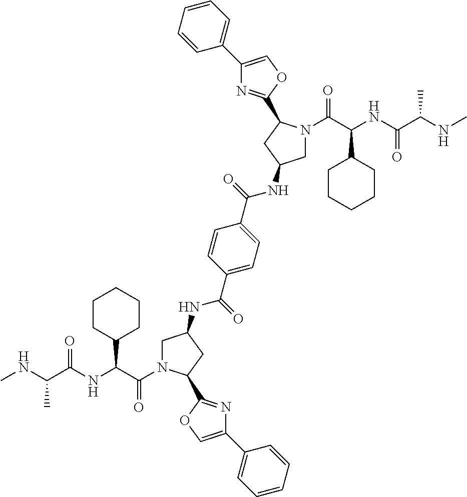

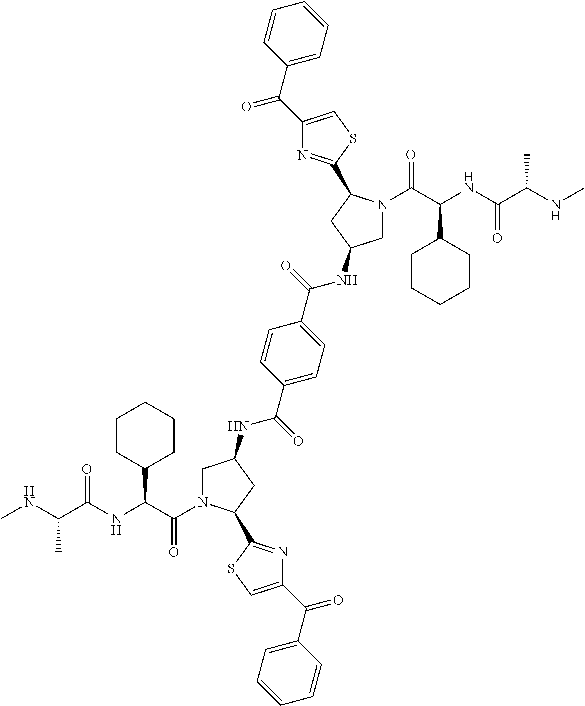

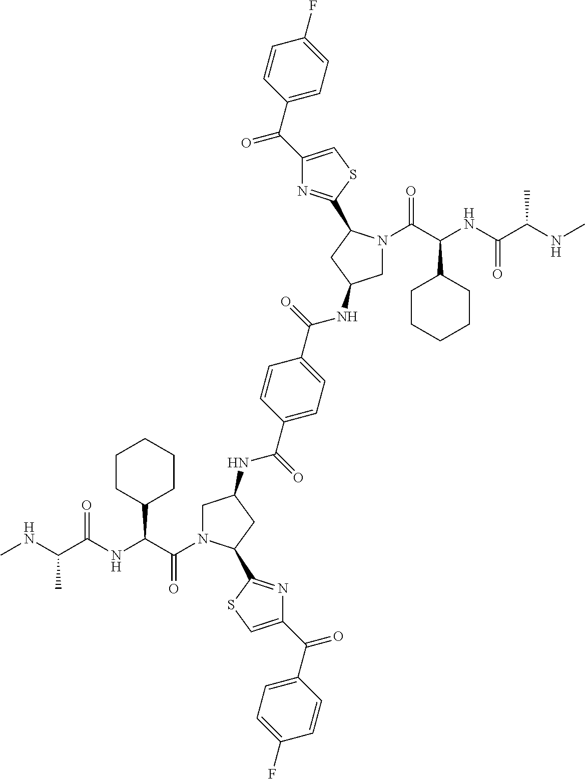

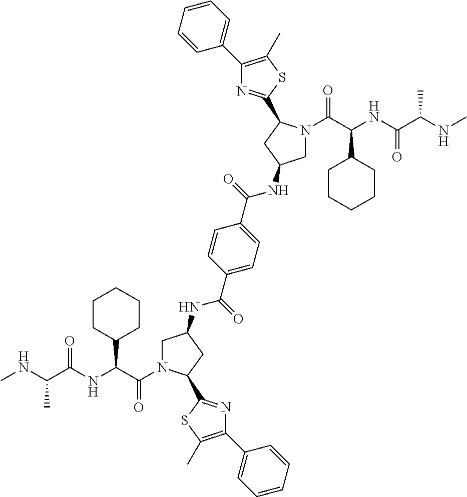

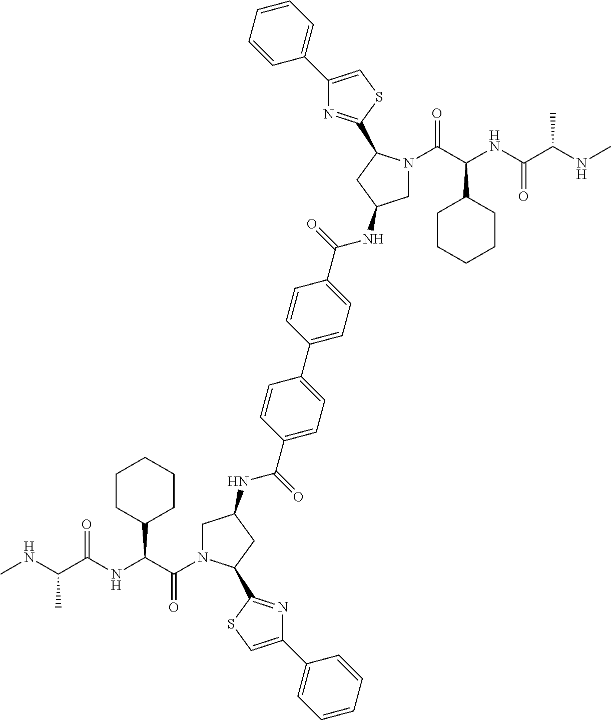

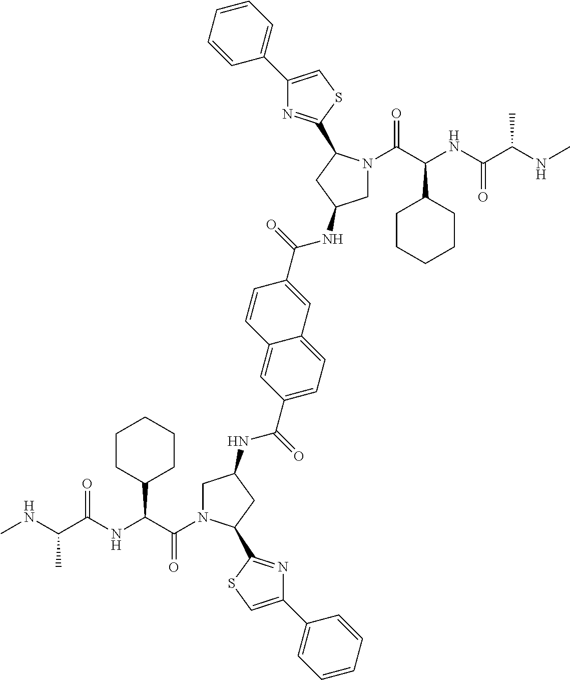

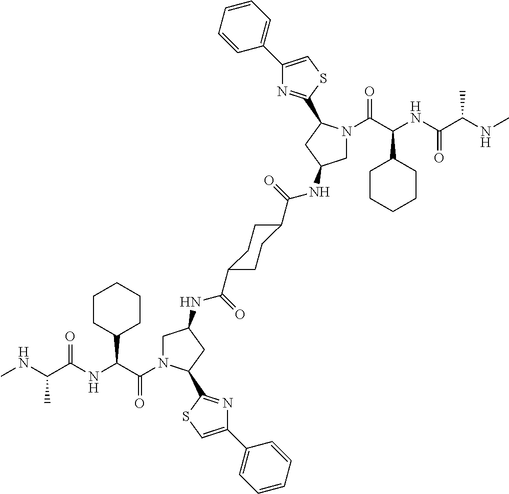

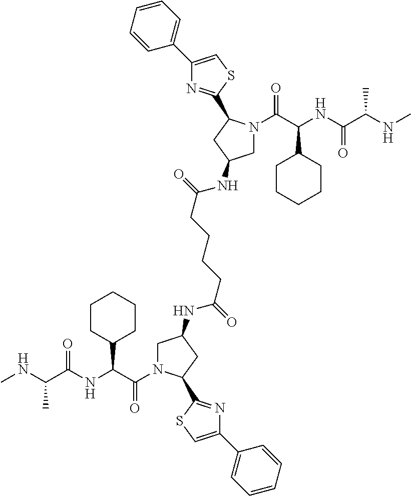

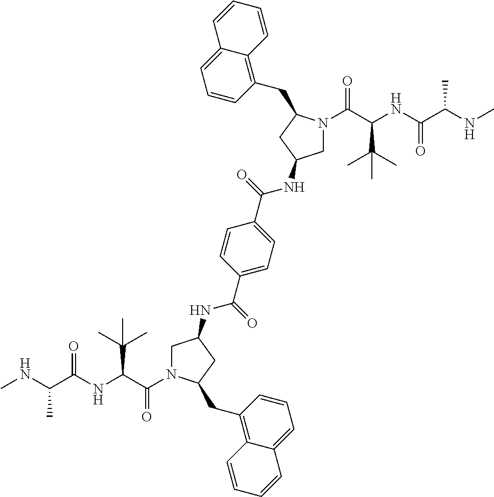

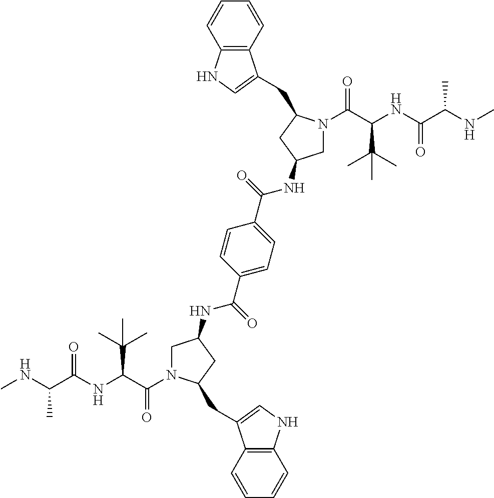

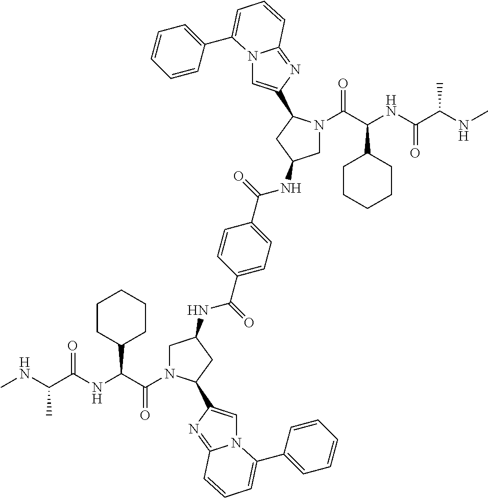

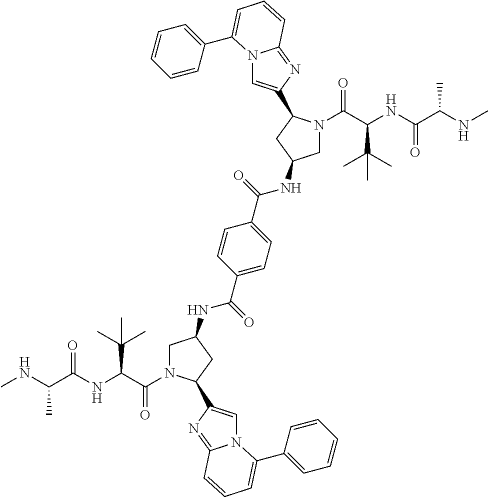

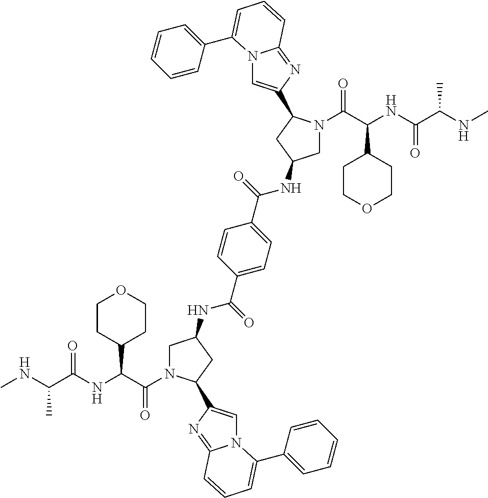

Representative compounds of the present invention which can be prepared by simple modification of the above procedures are illustrated below:

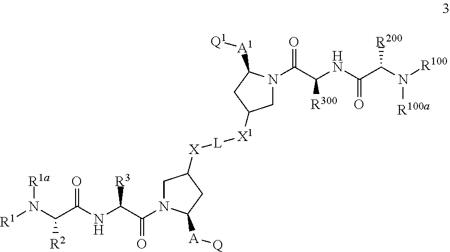

R¹, R^{1a}, R², R³, R⁴, R¹⁰⁰, R^{100a}, R²⁰⁰, R³⁰⁰, and R⁴⁰⁰ are defined as hereinabove —X-L-X¹— is chosen from, for example:

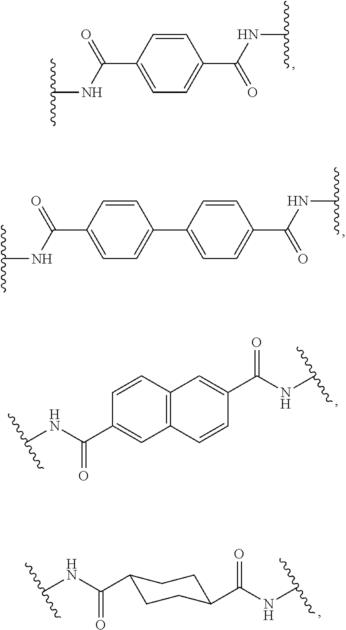

R³ and R³⁰⁰ are chosen from, for example:

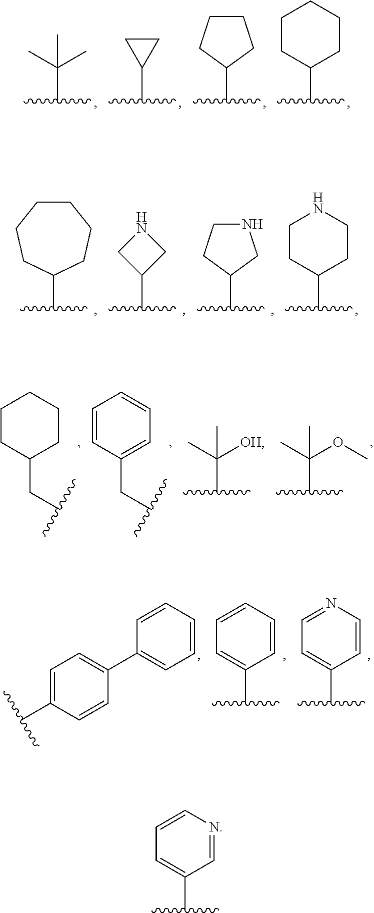

A and A′ are chosen from:
—(CH₂)ₙ—, n is 0 or 1.
Q and Q¹ are chosen from:

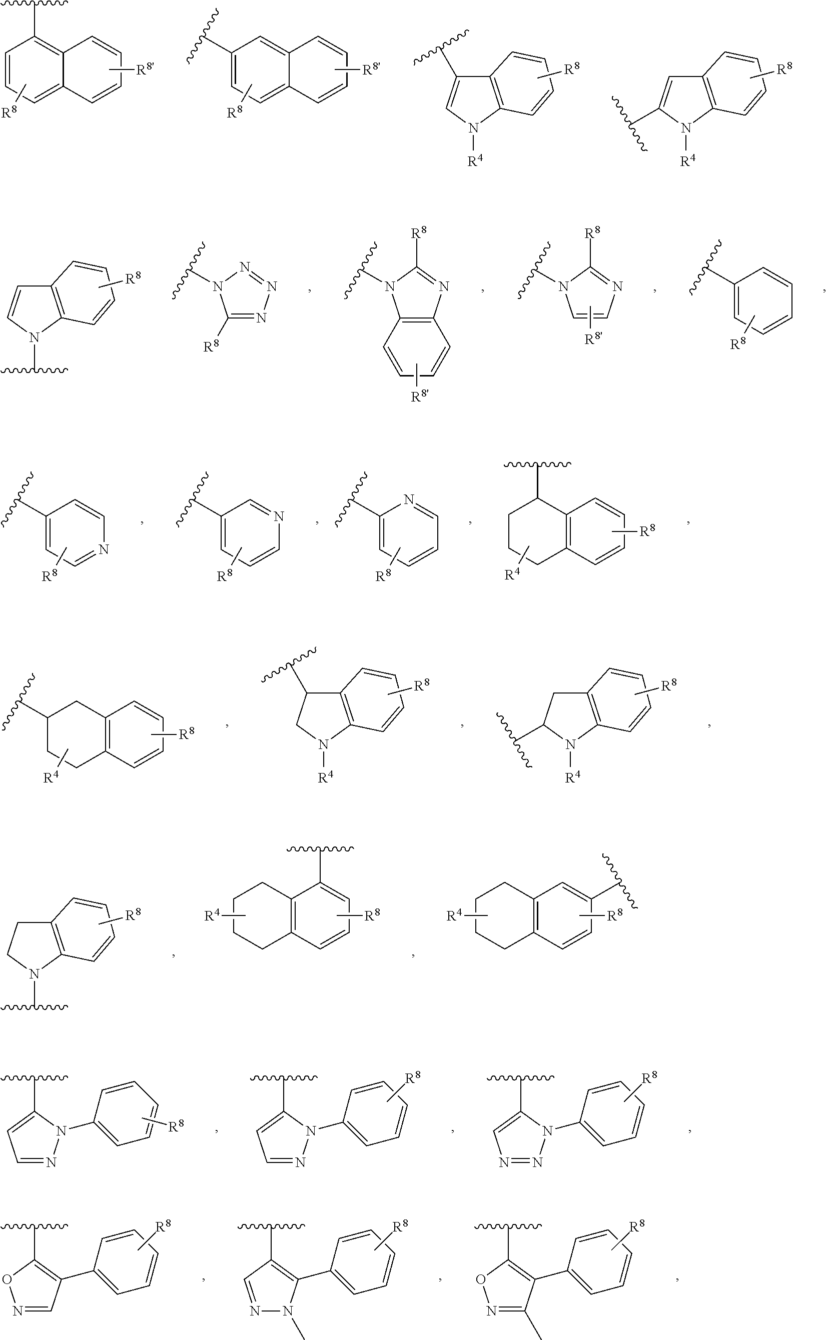

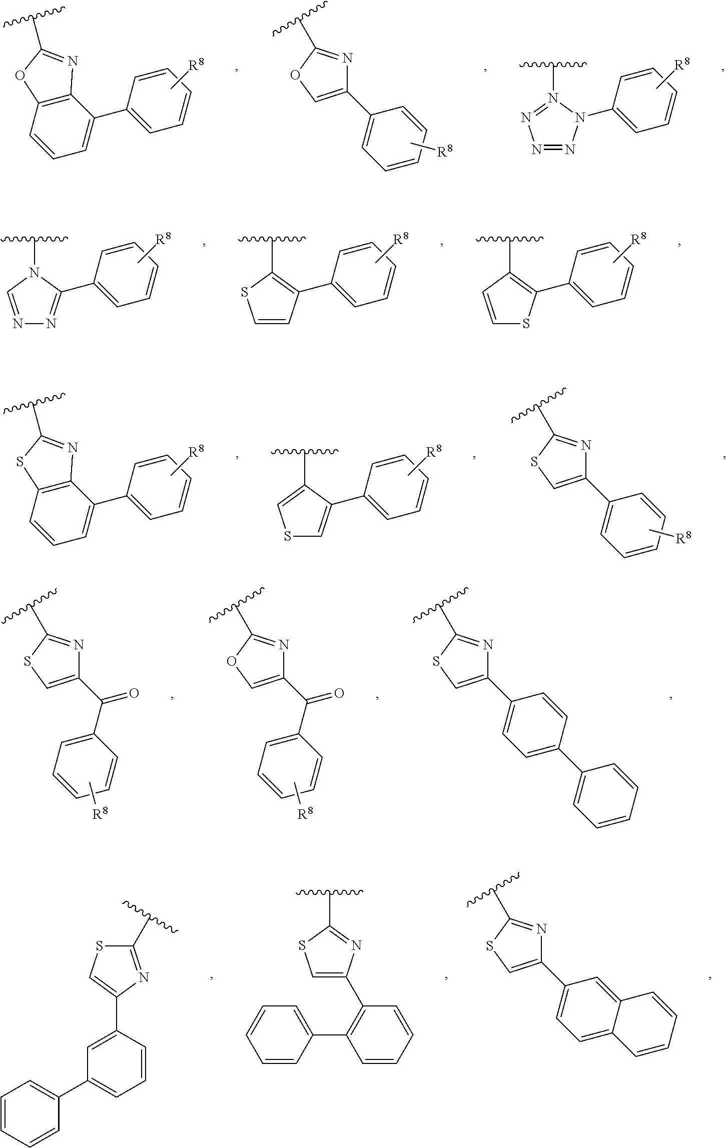

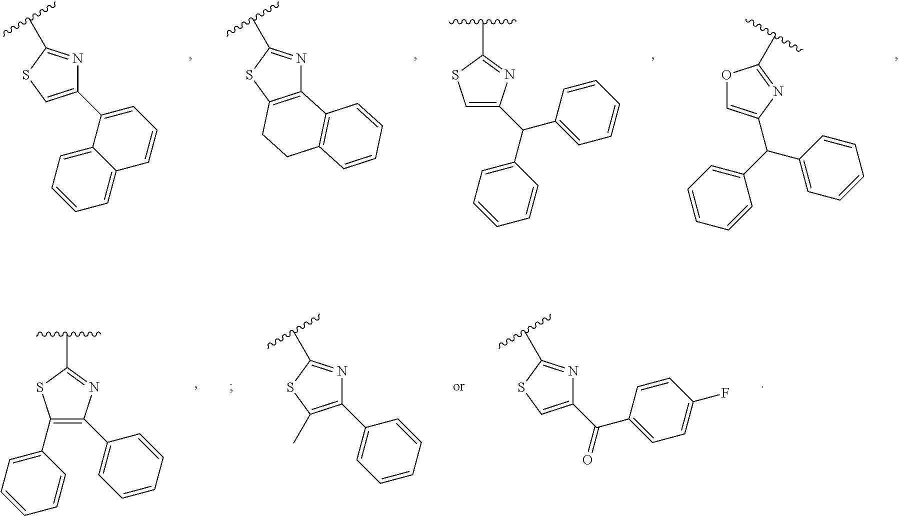

### Assays

### Molecular Constructs for Expression

GST-XIAP BIR3RING: XIAP coding sequence amino acids 246-497 cloned into PGEX2T1 via BamH1 and AVA I. The plasmid was transformed into E. coli DH5α for use in protein expression and purification.

GST-HIAP2 (cIAP-1) BIR 3: HIAP2 coding sequence from amino acids 251-363 cloned into PGex4T3 via BamH1 and XhoI. The plasmid was transformed into E. coli DH5α for use in protein expression and purification.

GST-HIAP1(cIAP-2) BIR 3: HIAP1 coding sequence from amino acids 236-349, cloned into PGex4T3 via BamH1 and XhoI. The plasmid was transformed into E. coli DH5α for use in protein expression and purification.

GST-linker BIR 2 BIR3Ring: XIAP coding sequence from amino acids 93-497 cloned into PGex4T1 via BamH1 and XhoI. Amino acids 93-497 were amplified from full length XIAP in pGex4t3, using the primers: TTAATAGGATCCATCAACGGCTTTTATC and GCTGCATGTGTGTCAGAGG, using standard PCR conditions. The PCR fragment was TA cloned into pCR-2.1 (invitrogen). Linker BIR 2 BIR 3Ring was subcloned into pGex4T1 by BamHI/XhoI digestion. The plasmid was transformed into E. coli DH5α for use in protein expression and purification.

Full-length human XIAP, AEG plasmid number 23. XIAP coding sequence amino acids 1-497 cloned into GST fusion vector, PGEX4T1 via BamH1 and Xho I restriction sites. The plasmid was transformed into E. coli DH5α for use in protein purification.

GST-XIAP linker BIR 2: XIAP linker BIR 2 coding sequence from amino acids 93-497 cloned into pGex4T3 via BamHI and XhoI. The plasmid was transformed into E. coli DH5α for use in protein expression and purification.

### Expression and Purification of Recombinant Proteins

### A. Expression of Recombinant Proteins

Glutathione S-transferase (GST) tagged proteins were expressed in Escherichia coli strains DH5-alpha. For expression of full length XIAP, individual or combinations of XIAP-BIR domains, cIAP-1, cIAP-2 and Livin transformed bacteria were cultured overnight at 37° C. in Luria Broth (LB) medium supplemented with 50 ug/ml of ampicillin. The overnight culture was then diluted 25 fold into fresh LB ampicillin supplemented media and bacteria were grown up to A₆₀₀=0.6 then induced with 1 mM isopropyl-D-1-thiogalactopyranoside for 3 hours. Upon induction, cells were centrifuged at 5000 RPM for 10 minutes and the media was removed. Each pellet obtained from a 1 liter culture received 10 ml of lysis buffer (50 mM Tris-HCl, 200 mM NaCl, 1 mM DTT, 1 mM PMSF, 2 mg/ml of lysosyme, 100 μg/ml)), was incubated at 4° C. with gentle shaking. After 20 minutes of incubation, the cell suspension was placed at −80° C. overnight or until needed.

### B. Purification of Recombinant Proteins

For purification of recombinant proteins, the IPTG-induced cell lysate was thawed vortexed and then disrupted by flash freezing in liquid nitrogen two times with vortexing after each thaw. The cells were disrupted further by passing the extract four times through a Bio-Neb Cell disruptor device (Glas-col) set at 100 psi with Nitrogen gas. The extract was clarified by centrifugation at 4° C. at 15000 RPM in a SS-34 Beckman rotor for 30 minutes. The resulting supernatant was then mixed with 2 ml of glutathione-Sepharose beads (Pharmacia) per 500 ml cell culture (per 1000 ml culture for full length XIAP) for 1 hour at 4° C. Afterwards, the beads were washed 3 times with 1× Tris-Buffered Saline (TBS) to remove unbound proteins. The retained proteins were eluted with 2 washes of 2 ml of 50 mM TRIS pH 8.0 containing 10 mM reduced glutathione. The eluted proteins were pooled and precipitated with 604 g/liter of ammonium sulfate and the resulting pellet re-suspended into an appropriate buffer. As judged by SDS-PAGE the purified proteins were >90% pure. The protein concentration of purified proteins was determined from the Bradford method.

His-tag proteins were expressed in the E. Coli strain in E. coli AD494 cells using a pet28ACPP32 construct. The soluble protein fraction was prepared as described above. For protein purification, the supernatant was purified by affinity chromatography using chelating-Sepharose (Pharmacia) charged with NiSO₄ according to the manufacturer's instructions. Purity of the eluted protein was >90% pure as determined by SDS-PAGE. The protein concentration of purified proteins was determined from the Bradford assay.

### Synthesis of Fluorescent Probe P1

A fluorescent peptide probe, Fmoc-Ala-Val-Pro-Phe-Tyr(t-Bu)-Leu-Pro-Gly(t-Bu)-Gly-OH was prepared using standard Fmoc chemistry on 2-chlorotrityl chloride resin (see Int. J. Pept. Prot. Res. 38:555-561, 1991). Cleavage from the resin was performed using 20% acetic acid in dichloromehane (DCM), which left the side chain still blocked. The C-terminal protected carboxylic acid was coupled to 4′-(aminomethy)fluorescein (Molecular Probes, A-1351; Eugene, Oreg.) using excess diisopropylcarbodiimide (DIC) in dimethylformamide (DMF) at room temperature and was purified by silica gel chromatography (10% methanol in DCM). The N-terminal Fmoc protecting group was removed using piperidine (20%) in DMF, and purified by silica gel chromatography (20% methanol in DCM, 0.5% HOAc). Finally, the t-butyl side chain protective groups were removed using 95% trifluoroacetic acid containing 2.5% water and 2.5% triisopropyl silane, to provide probe P1 (>95% pure, HPLC).

### Probe P2

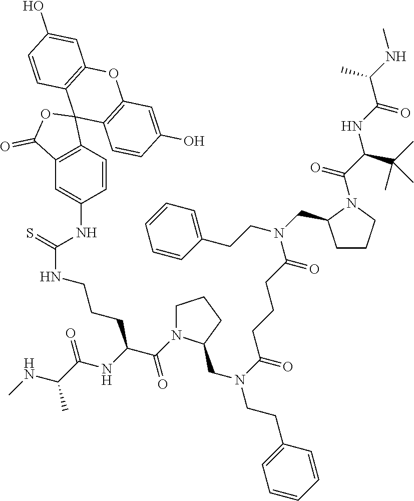

Probe P2 was prepared using methods as described in WO 2007/131,366.

### Binding Assay

### Fluorescence Polarization-Based Competition Assay

For all assays, the fluorescence and fluorescence-polarization was evaluated using a Tecan Polarion instrument with the excitation filter set at 485 nm and the emission filter set at 535 nm. For each assay, the concentration of the target protein was first established by titration of the selected protein in order to produce a linear dose-response signal when incubated alone in the presence of the fluorescent probe P1 or P2. Upon establishing these conditions, the compounds potency (IC₅₀) and selectivity, was assessed in the presence of a fix defined-amount of target protein and fluorescent probe and a 10 point serial dilution of the selected compounds. For each IC₅₀ curve, the assays were run as followed: 25 uL/well of diluted compound in 50 mM MES buffer pH 6.5 was added into a black 96 well plate then 25 ul/well of bovine serum albumin (BSA) at 0.5 mg/ml in 50 mM MES pH 6.5. Auto-fluorescence for each compound was first assessed by performing a reading of the compound/BSA solution alone. Then 25 uL of the fluorescein probe (P1 or P2) diluted into 50 mM MES containing 0.05 mg/ml BSA were added and a reading to detect quenching of fluorescein signal done. Finally 25 uL/well of the target or control protein (GST-BIRs) diluted at the appropriate concentration in 50 mM MES containing 0.05 mg/ml BSA were added and the fluorescence polarization evaluated.

### Determination of IC₅₀ and Inhibitory Constants

For each assay the relative polarization-fluorescence units were plotted against the final concentrations of compound and the IC₅₀ calculated using the Grad pad prism software and/or Cambridge soft. The ki value were derived from the calculated IC₅₀ value as described above and according to the equation described in Nikolovska-Coleska, Z. (2004) Anal Biochem 332, 261-273.

### Fluorescence Polarization Competition Assay

The kᵢ of various compounds in the BIR2-BIR3-ring FP assay, using probe P2, was determined to be less than 10 uM against BIR2/BIR3XIAP, cIAP1 or cIAP2

### Cell Culture and Cell Death Assays

### A. Cell Culture

SKOV3 (ovarian) and HCT-116 (colon) cancer cells were cultured in RPMI1640 media supplemented with 10% FBS and 100 units/mL of Penicillin and Steptomycin.

### B. Assays

Survival assays were performed on various cell lines including SKOV3 and HCT-116 cells. Cells were seeded in 96 well plates at a respective density of 2000 and 5000 cells per well and incubated at 37° C. in presence of 5% CO₂ for 24 hours. Selected compounds were diluted into the media at various concentration ranging from 0.01 uM up to 100 uM. Diluted compounds were added onto the SKOV3 cells. For the HCT-116 cells, the compounds were added followed by 1-3 ng/ml of agonist TRAIL receptor antibody (ETR1). After 72 hours cellular viability was evaluated by MTS based assays. A solution of [3-(4,5-dimethylthiazol-2-yl)-5-(3-carboxymethoxyphenyl)-2-(4-sulfophenyl)-2H-tetrazolium, inner salt; MTS] was added onto cells for a period of 1 to 4 hours. Upon incubation the amount of converted MTS was evaluated using a Tecan spectrophotometer set at 570 nm.

SKOV3 cells were treated with selected compounds of the present invention and found to have EC₅₀s of 1 uM or less.

HCT116 cells in the presence of agonist TRAIL receptor antibody (ETR1) were treated with selected compounds of the present invention and shown to have EC₅₀s of 1 uM or less.

### Survival MTT Assay

One day prior the treatment with compound, 2000 to 5000 cells per well were plated in a tissue culture treated 96 well format dish with 100 ul of media and incubated at 37° C., 5% CO₂. On the day of compound treatment, compounds were diluted with cell culture media to a working stock concentration of 2×. 100 uL of diluted compound were then added to each well. The treated plate was incubated for 72 h at 37° C., 5% CO₂. Upon incubation, the cell viability was assessed as followed 20 uL of MTT reagent at 5 mg/ml were added per well to cell plate. The plate was incubated for 2 h at 37° C. in presence of 5% CO₂. The supernatant was then removed from the plate and 100 uL of isopropanol were added. The absorbance was measured in a TECAN spectrophotometer at 570 nm. The percentage of viability was expressed in percentage of the signal obtained with non treated cells.

### Other Embodiments

From the foregoing description, it will be apparent to one of ordinary skill in the art that variations and modifications may be made to the invention described herein to adapt it to various usages and conditions. Such embodiments are also within the scope of the present invention.

1. A compound of Formula 1: 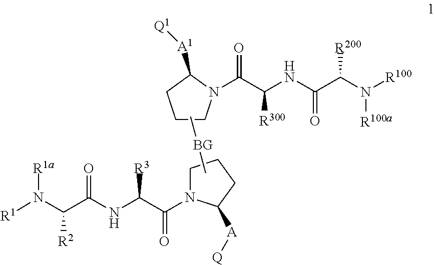
or a salt thereof,
wherein
n is 0, 1, or 2;
m is 0, 1 or 2;
Y is NH, O or S;
BG is —X-L-X¹—;
X and X¹ are independently
1) O,
2) NR¹¹,
3) S,
4) —C₁-C₆ alkyl-,
5) —C₁-C₆ alkyl-O—,
6) —C₁-C₆ alkyl-NR¹¹—,
7) —C₁-C₆ alkyl-S—, 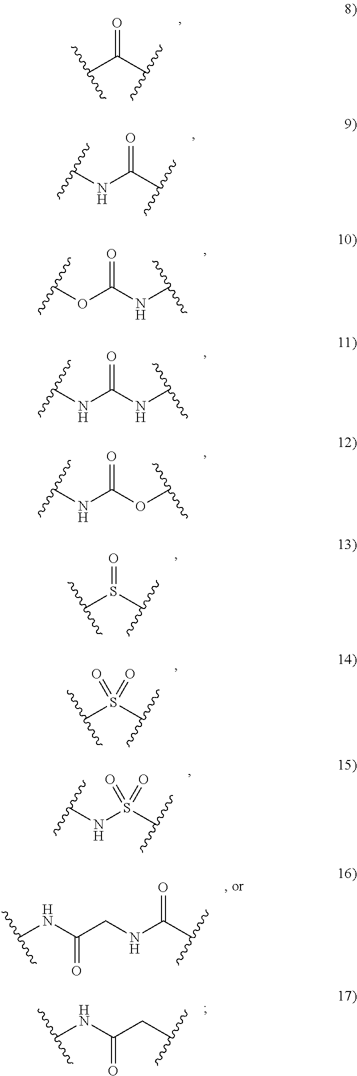
L is
1) —C₁-C₂₀ alkyl-,
2) —C₂-C₆ alkenyl-,
3) —C₂-C₄ alkynyl-,
4) —C₃-C₇ cycloalkyl-,
5) -aryl-,
6) -biphenyl-,
7) -heteroaryl-,
8) -biheteroaryl-,
9) -heterocyclyl-,
10) —C₁-C₆ alkyl-(C₂-C₆ alkenyl)-C₁-C₆ alkyl-,
11) —C₁-C₆ alkyl-(C₂-C₄ alkynyl)-C₁-C₆ alkyl-,
12) —C₁-C₆ alkyl-(C₃-C₇ cycloalkyl)-C₁-C₆ alkyl-,
13) —C₁-C₆ alkyl-aryl-C₁-C₆ alkyl-,
14) —C₁-C₆ alkyl-biphenyl-C₁-C₆ alkyl-,
15) —C₁-C₆ alkyl-heteroaryl-C₁-C₆ alkyl-,
16) —C₁-C₆ alkyl-heterocycyl-C₁-C₆ alkyl-,
17) —C₁-C₆ alkyl-Y—C₁-C₆ alkyl-,
18) -aryl-Y-aryl-,
19) -heteroaryl-Y-heteroaryl-,
20) -heterocyclyl-Y-heterocyclyl-, 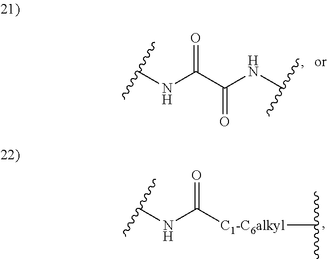
wherein the alkyl, alkenyl, alkynyl, and cycloalkyl are optionally substituted with one or more R⁴ substituents, and the aryl, biphenyl, heteroaryl, and heterocyclyl are optionally substituted with one or more R⁸ substituents;
A and A¹ are independently
1) —(CH₂)ₙ—,
2) —CH(C₁-C₆ alkyl)-,
3) —CH(C₃-C₇ cycloalkyl)-,
4) —C₃-C₇ cycloalkyl-, or
5) —CH(C₁-C₆ alkyl-C₃-C₇ cycloalkyl)-;
Q and Q¹ are independently
1) aryl,
2) heteroaryl,
3) heterocyclyl,
4) heterobicyclyl,
5) —X²-aryl,
6) —X²-heteroaryl;
7) —X²-heterocyclyl, or
8) —X²-heterobicyclyl,
wherein the aryl, heteroaryl, heterocyclyl, and heterobicyclyl are optionally substituted with one or more R⁸ substituents;
X² is
1) —O—,
2) —NR¹¹—,
3) —S(O)ₘ—, or
4) —C(O)—;
R¹, R^{1a}, R¹⁰⁰ and R^{100a} are independently
1) H, or
2) C₁-C₆ alkyl optionally substituted with one or more R⁴ substituents;
R² and R²⁰⁰ are independently
1) H, or
2) C₁-C₆ alkyl optionally substituted with one or more R⁴ substituents;
R³ and R³⁰⁰ are independently
1) H,
2) C₁-C₆ alkyl,
3) C₃-C₇ cycloalkyl,
4) C₃-C₇ cycloalkenyl,
5) aryl,
6) biphenyl,
7) heteroaryl,
8) heterocyclyl, or
9) heterobicyclyl,
wherein the alkyl, cycloalkyl, and cycloalkenyl are optionally substituted with one or more R⁴ substituents; and wherein the aryl, biphenyl, heteroaryl, and heterobicyclyl are optionally substituted with one of more R⁸ substituents;
R⁴ is
1) halogen,
2) NO₂,
3) CN,
4) haloalkyl,
5) C₁-C₆ alkyl,
6) C₂-C₆ alkenyl,
7) C₂-C₄ alkynyl,
8) C₃-C₇ cycloalkyl,
9) C₃-C₇ cycloalkenyl,
10) aryl,
11) heteroaryl,
12) heterocyclyl,
13) heterobicyclyl,
14) —OR⁵,
15) —S(O)ₘR⁵,
16) —NR⁶R⁷,
17) —NR⁶S(O)₂R⁹,
18) —C(O)R⁵,
19) —C(O)OR⁵,
20) —CONR⁶R⁷,
21) —S(O)₂NR⁶R⁷
22) —OC(O)R⁵,
23) —OC(O)Y—R⁹,
24) —SC(O)R⁵, or
25) —NC(Y)NR⁶R⁷,
wherein the aryl, heteroaryl, heterocyclyl, and heterobicyclyl are optionally substituted with one or more R⁸ substituents;
R⁵ is
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) R⁶R⁷NC(═Y),
13) C₁-C₆ alkyl-C₂-C₄ alkenyl, or
14) C₁-C₆ alkyl-C₂-C₄ alkynyl,
wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;
R⁶ and R⁷ are each independently
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) —C(O)R⁹,
13) —C(O)Y—R⁹, or
14) —S(O)₂—R⁹,
wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;
or R⁶ and R⁷ together with the nitrogen atom to which they are bonded form a five, six or seven membered heterocyclic ring optionally substituted with one or more R⁴ substituents;
R⁸ is
1) halogen,
2) NO₂,
3) CN,
4) C₁-C₆ alkyl,
5) C₂-C₆ alkenyl,
6) C₂-C₄ alkynyl,
7) C₃-C₇ cycloalkyl,
8) C₃-C₇ cycloalkenyl,
9) haloalkyl,
10) —OR⁵,
11) —NR⁶R⁷,
12) —SR⁵,
13) —C(O)R⁵,
14) —C(O)OR⁵,
15) —S(O)ₘR⁵,
16) —CONR⁶R⁷,
17) —S(O)₂NR⁶R⁷,
18) aryl,
19) heteroaryl,
20) heterocyclyl, or
21) heterobicyclyl,
wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;
R⁹ is
1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) heteroaryl,
9) heterocyclyl, or
10) heterobicyclyl,
wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents;
R¹⁰ is
1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) heteroaryl,
9) heterocyclyl,
10) heterobicyclyl,
11) C(O)—R⁹,
12) C(O)O—R⁹,
13) C(O)NR⁶R⁷,
14) S(O)ₘ—R⁹, or
15) C(═Y)NR⁶R⁷,
wherein the alkyl, alkenyl, alkynyl, cycloalkyl, and cycloalkenyl are optionally substituted with one or more R⁴ substituents; and wherein the aryl, heteroaryl, heterocyclyl, and heterobicyclyl are optionally substituted with one or more R⁸ substituents.

2. The compound of claim 1, wherein R⁸ is
1) halogen,
2) NO₂,
3) CN,
4) C₁-C₆ alkyl,
5) C₂-C₆ alkenyl,
6) C₂-C₄ alkynyl,
7) C₃-C₇ cycloalkyl,
8) C₃-C₇ cycloalkenyl,
9) haloalkyl,
10) —OR⁵,
11) —NR⁶R⁷,
12) —SR^{S},
13) —C(O)R⁵,
14) —C(O)OR⁵,
15) —S(O)ₘR⁵,
16) —CONR⁶R⁷,
17) —S(O)₂NR⁶R⁷,
18) aryl,
19) heteroaryl,
20) heterocyclyl, or
21) heterobicyclyl,
wherein the alkyl, alkenyl, alkynyl, cycloalkyl, and cycloalkenyl are optionally substituted with one or more R⁴ substituents;

3. The compound of claim 1, wherein A and A¹ are —(CH₂)ₙ— and n is 0.

4. The compound of claim 1, wherein A and A¹ are —(CH₂)ₙ— and n is 1.

5. The compound of claim 1, wherein Q and Q¹ are independently
1) aryl,
2) heteroaryl,
3) heterocyclyl,
4) heterobicyclyl,
wherein the aryl, heteroaryl, heterocyclyl, and heterobicyclyl are optionally substituted with one or more R⁸ substituents.

6. The compound of claim 1, wherein Q and Q¹ are

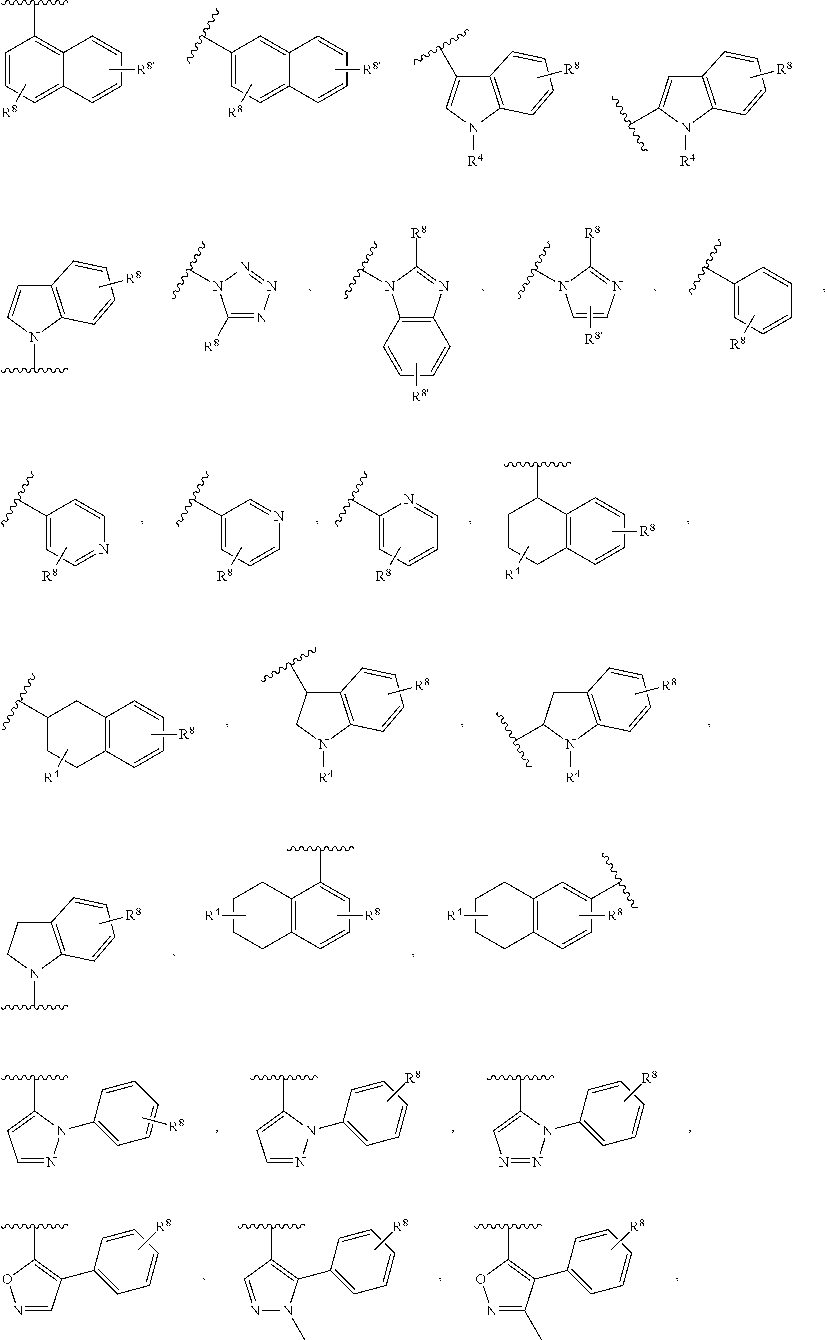

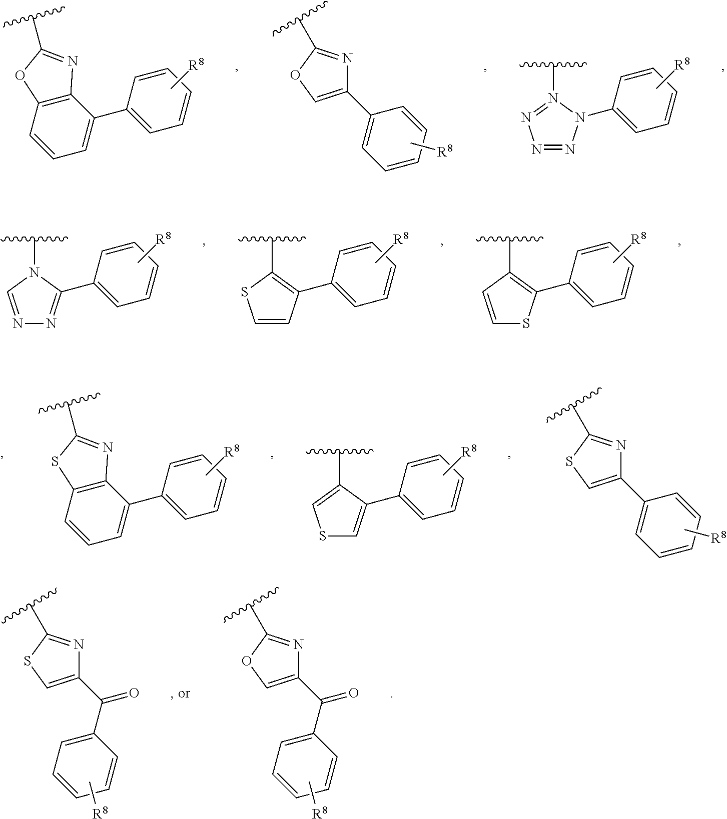

7. The compound of claim 1, wherein Q and Q¹ are:

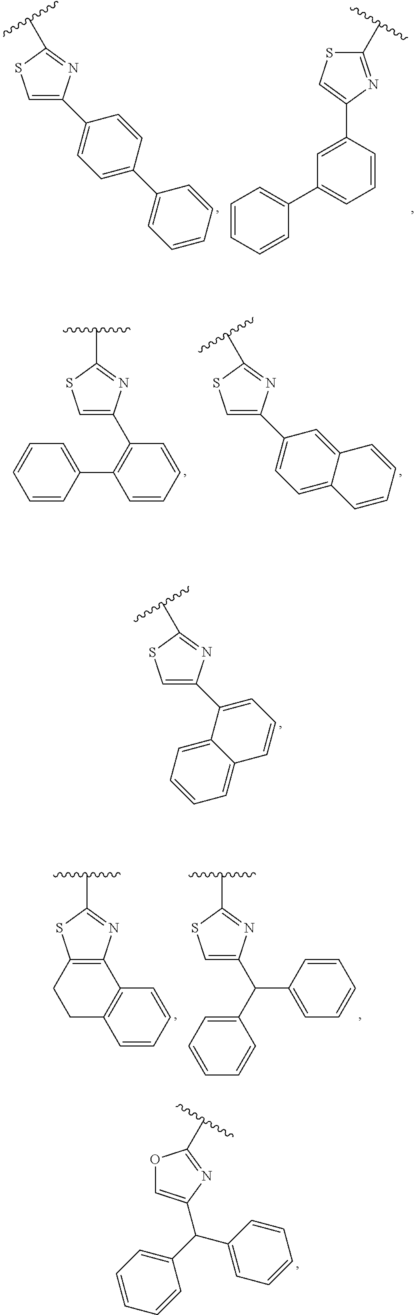

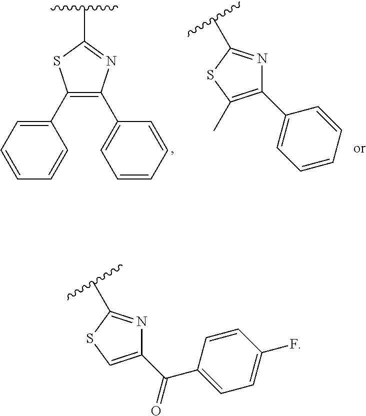

8. The compound of claim 1, wherein BG is

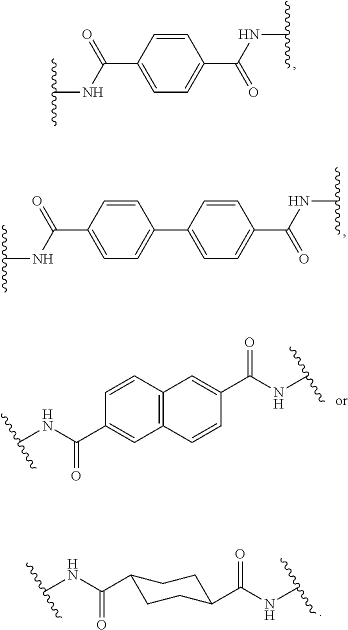

9. The compound of claim 1, wherein R³ and R³⁰⁰ are:

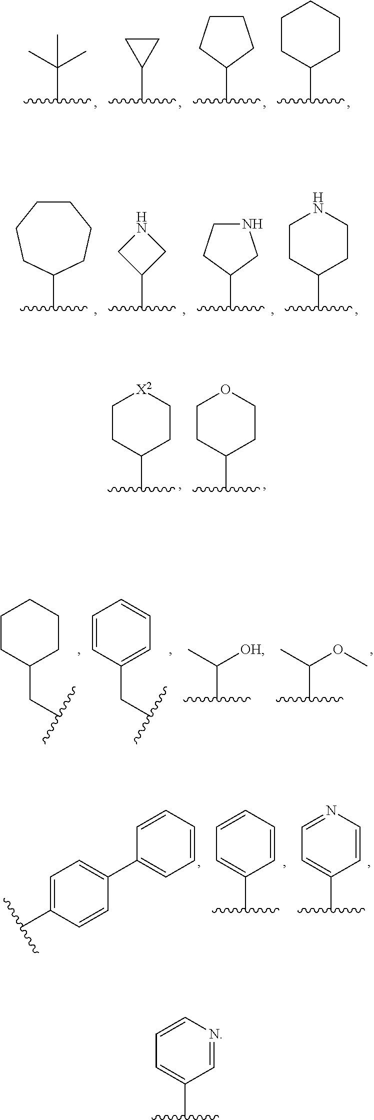

10. The compound of claim 1, wherein the compound is:

| COMPOUND | STRUCTURE |
|---|---|
| 1 | 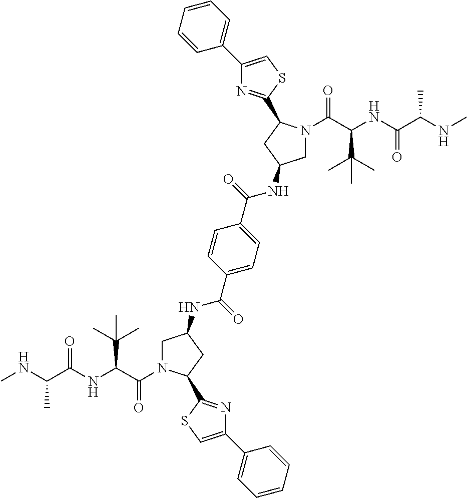 |
| 2 | 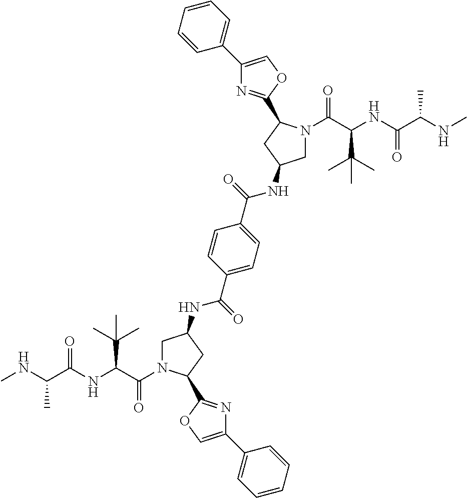 |
| 3 | 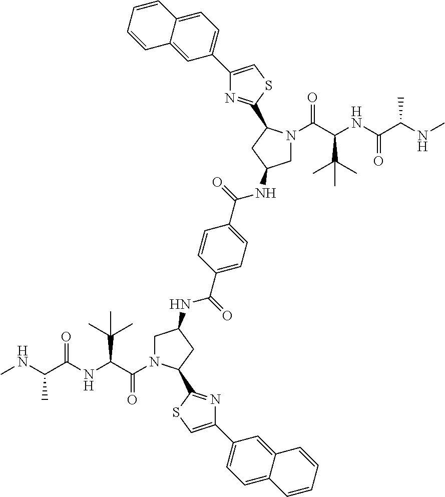 |
| 4 | 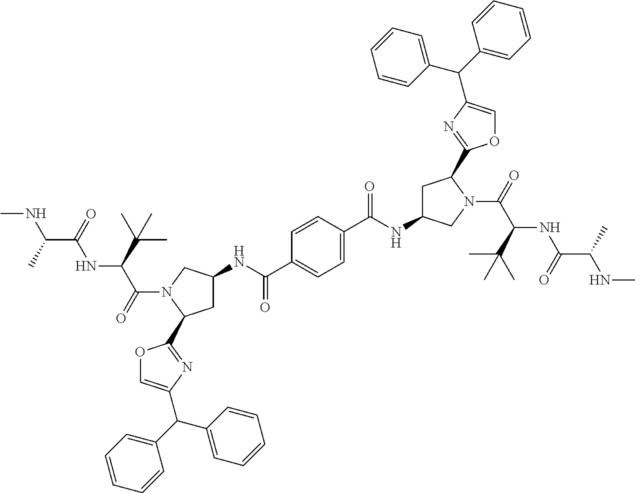 |
| 5 | 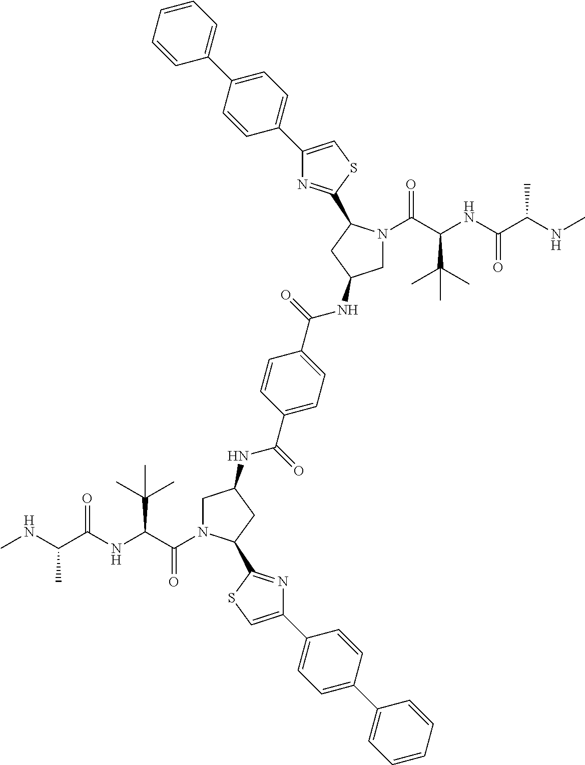 |
| 6 | 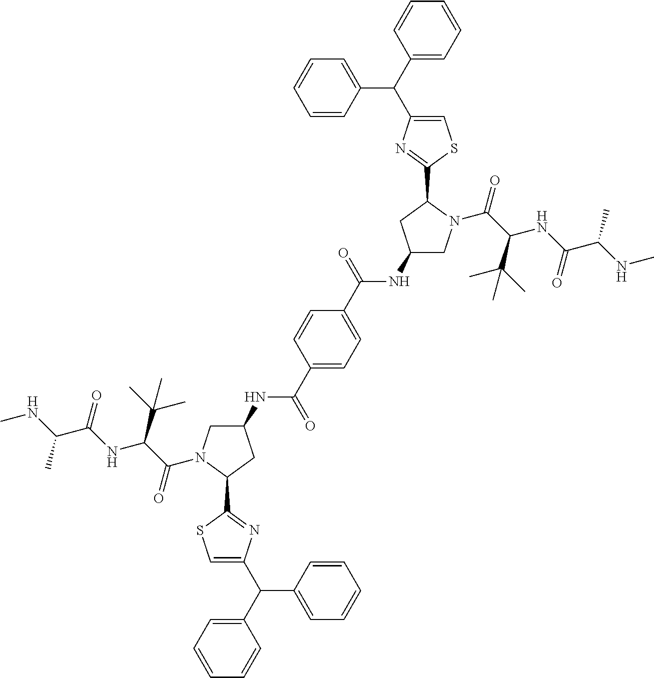 |
| 7 | 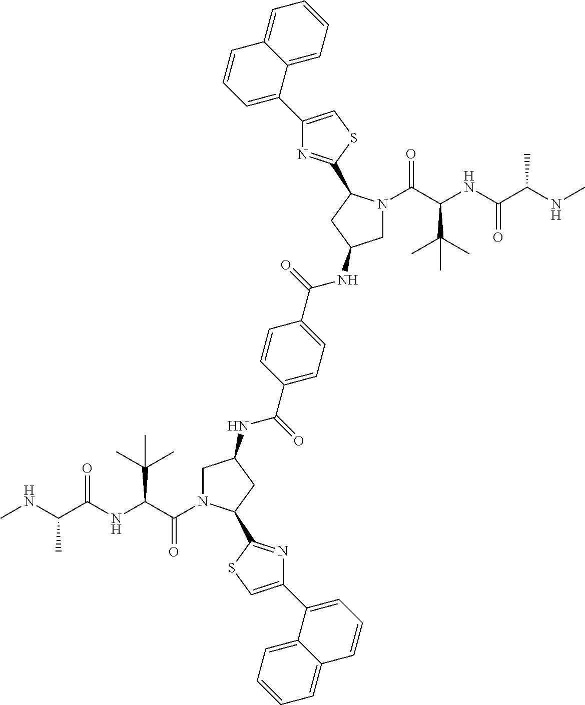 |
| 8 | 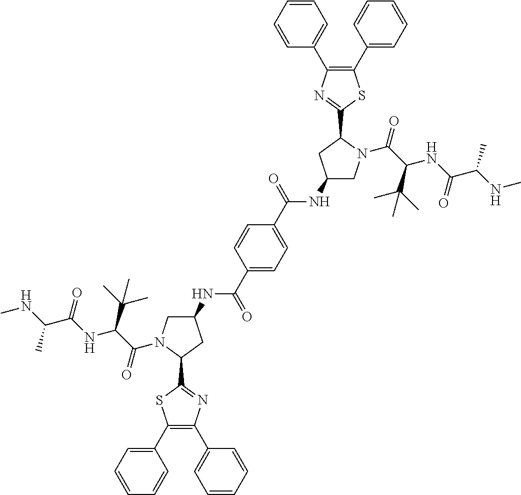 |
| 9 | 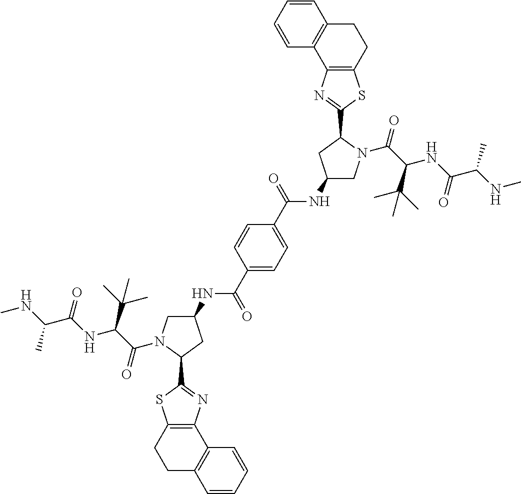 |
| 10 | 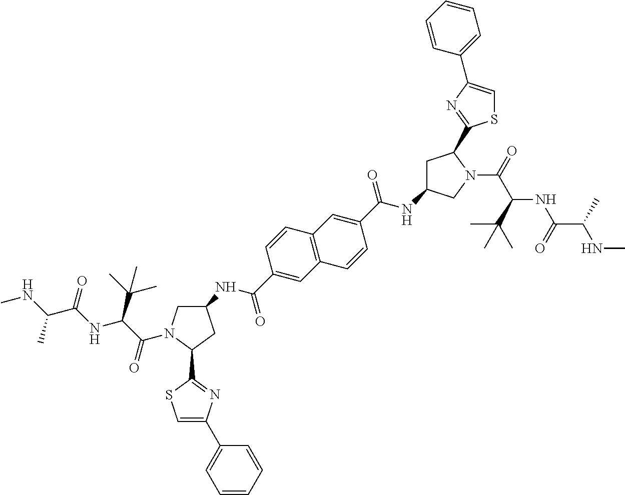 |
| 11 | 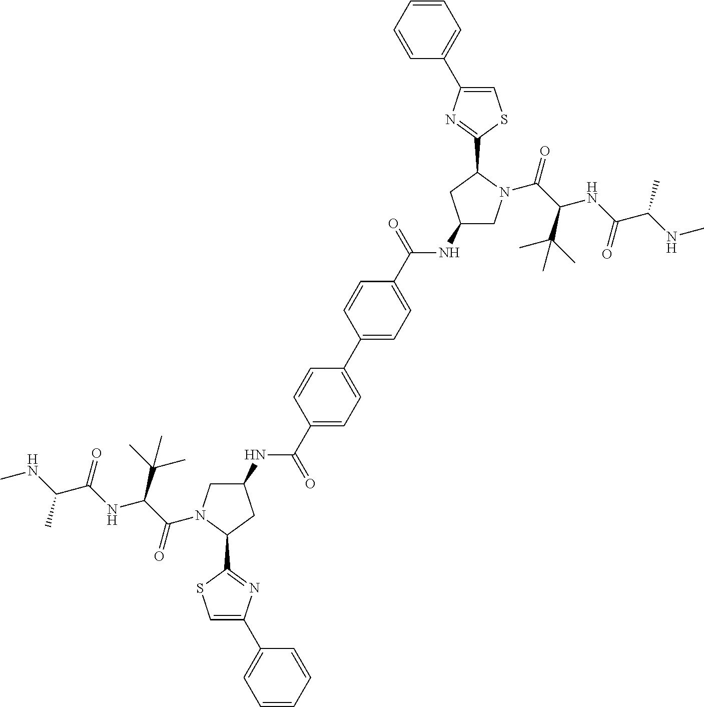 |
| 12 | 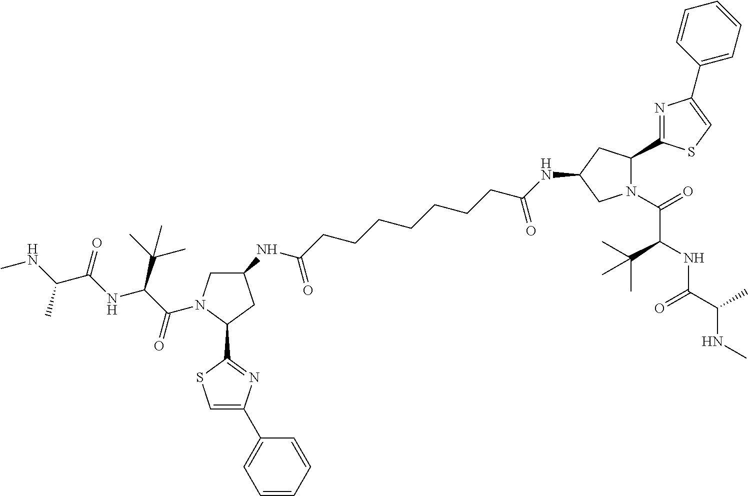 |
| 13 | 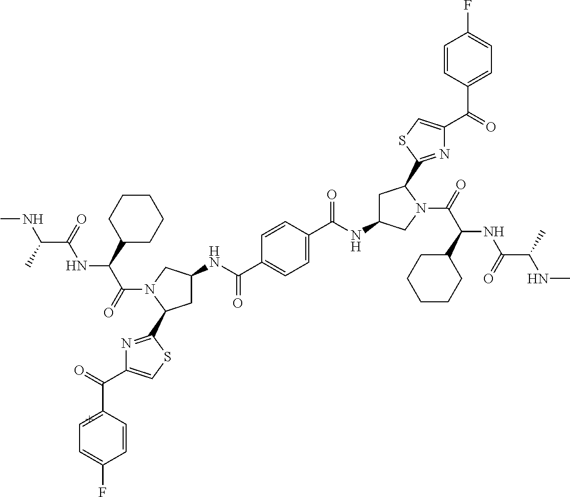 |
| 14 | 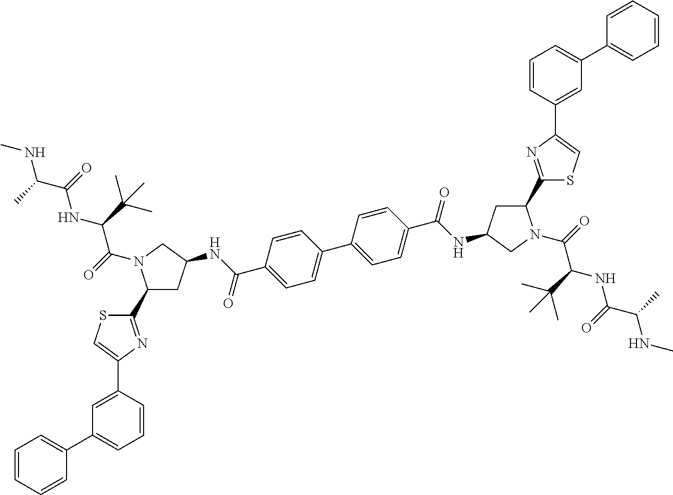 |
| 15 | 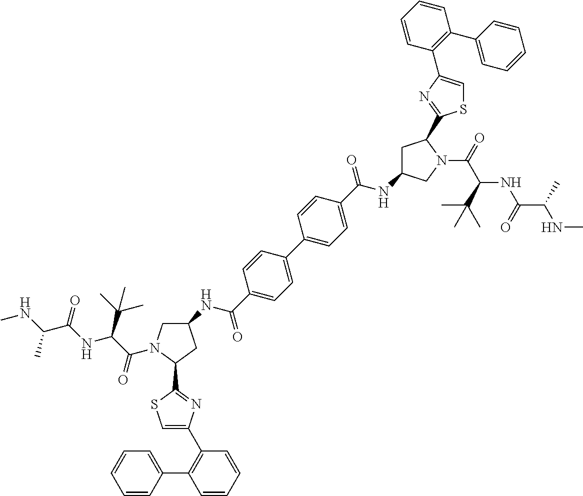 |
| 16 | 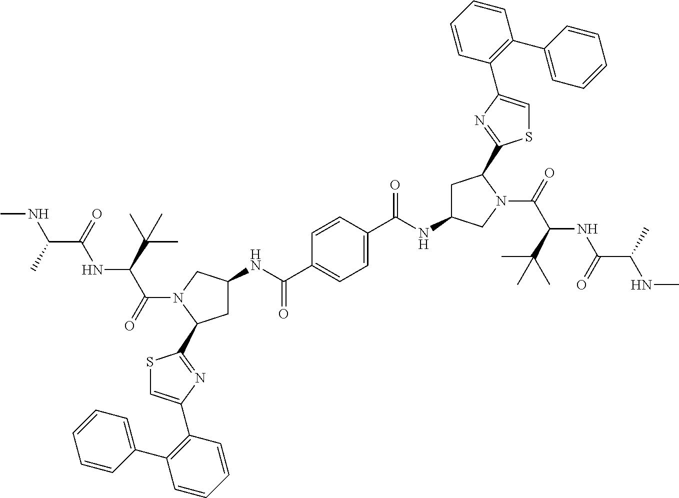 |
| 17 | 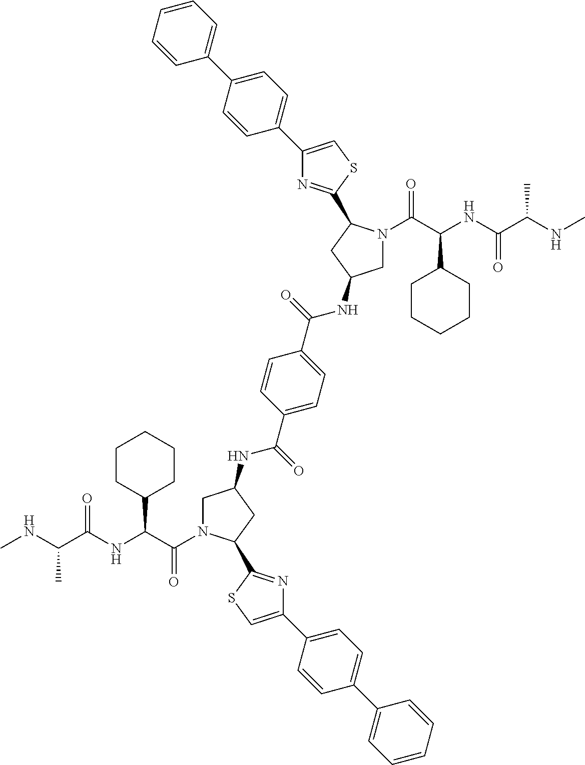 |
| 18 | 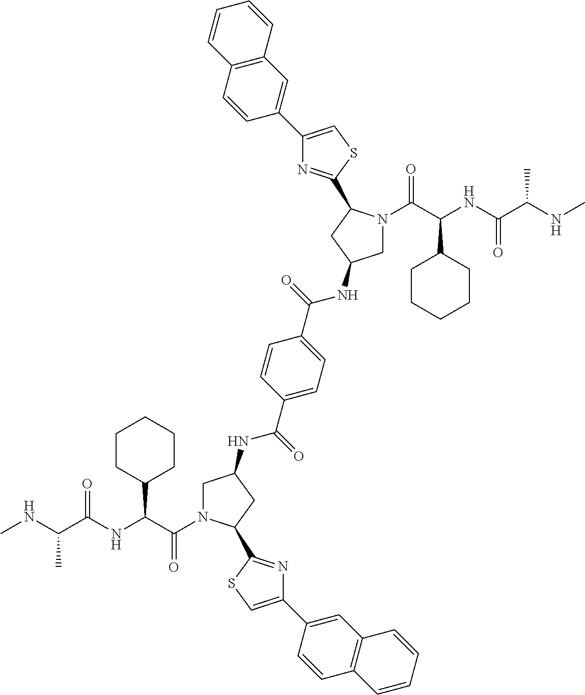 |
| 19 | 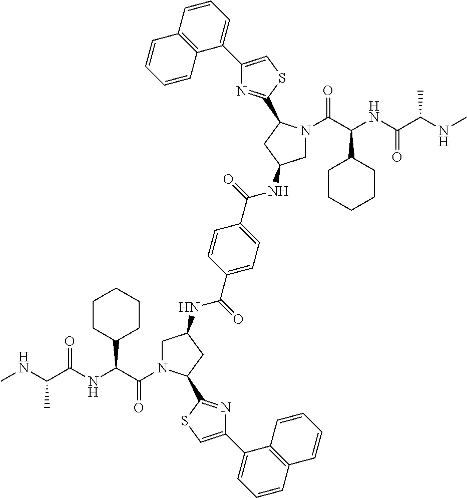 |
| 20 | 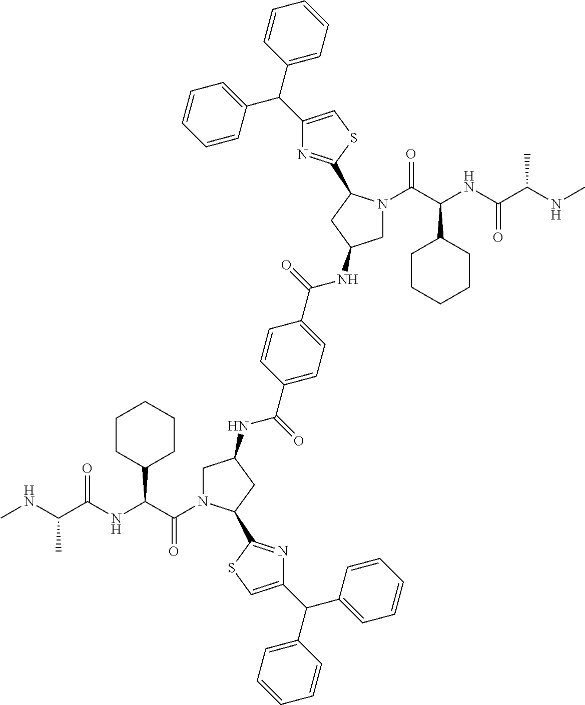 |
| 21 | 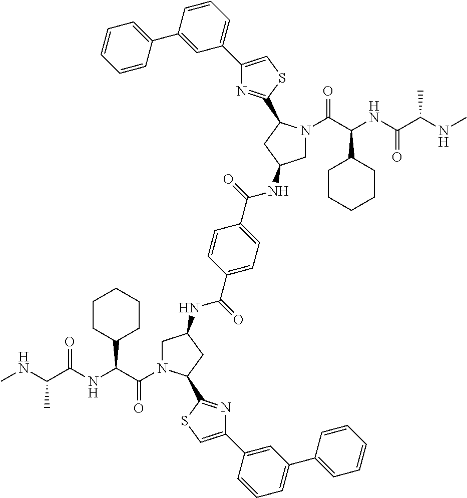 |
| 22 | 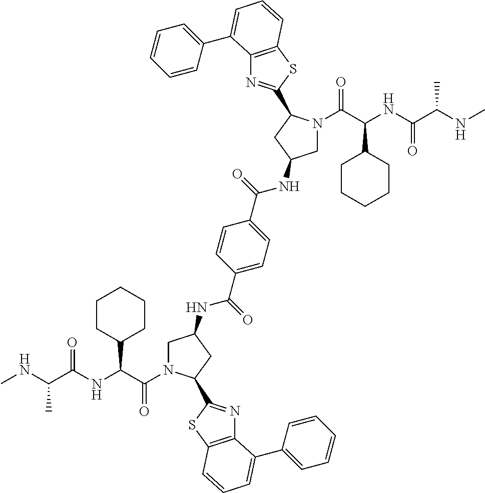 |
| 23 | 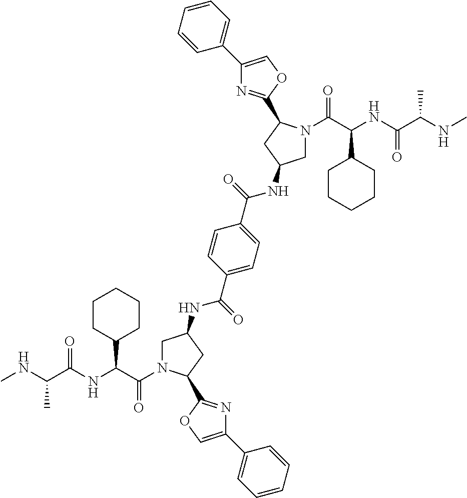 |
| 24 | 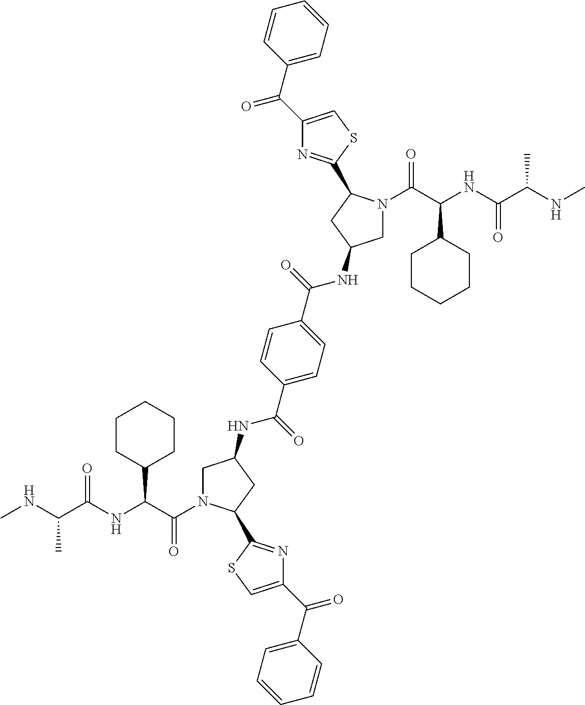 |
| 25 | 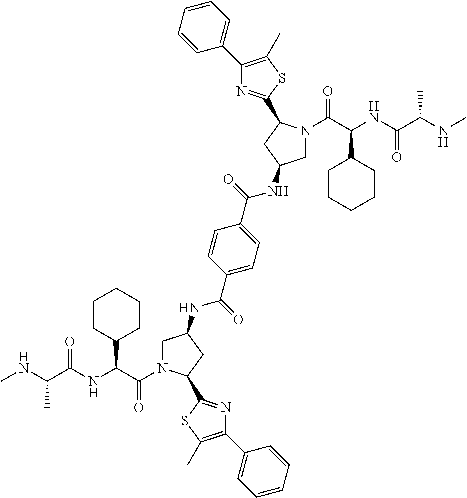 |
| 26 | 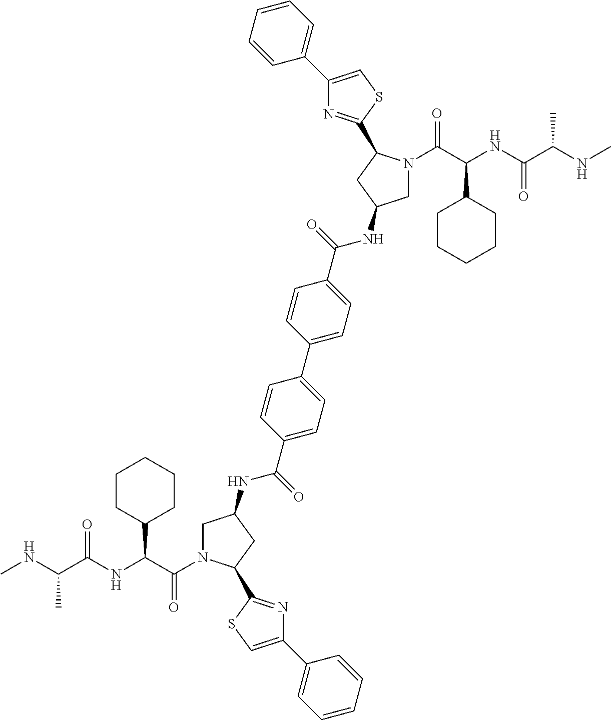 |
| 27 | 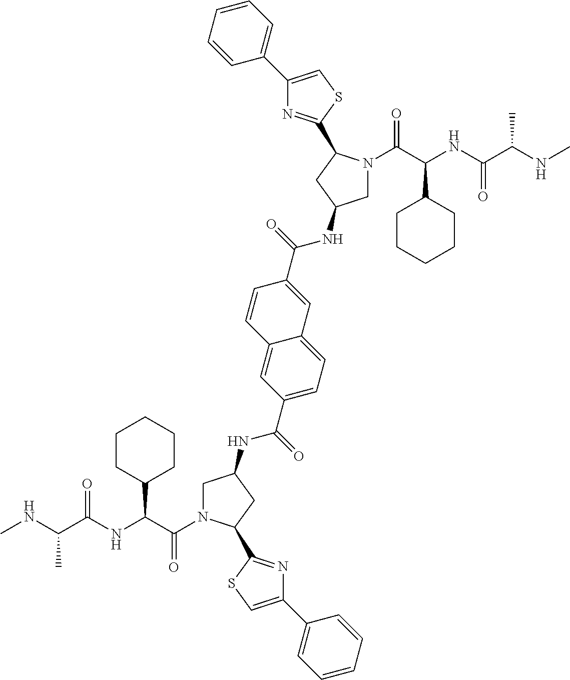 |
| 28 | 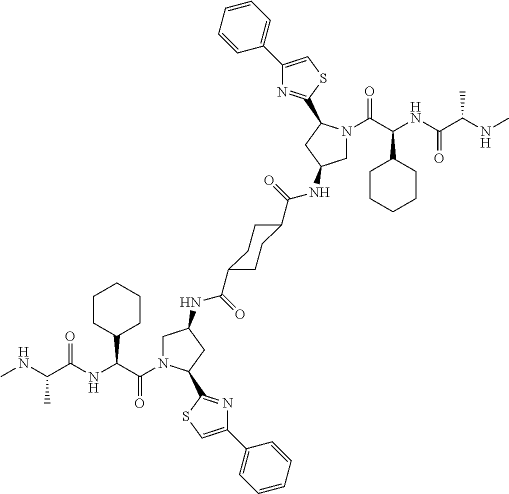 |
| 29 | 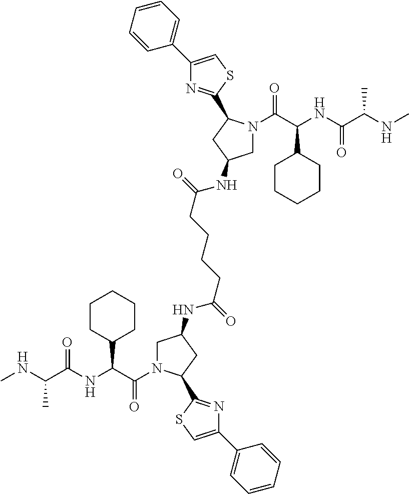 |
| 30 | 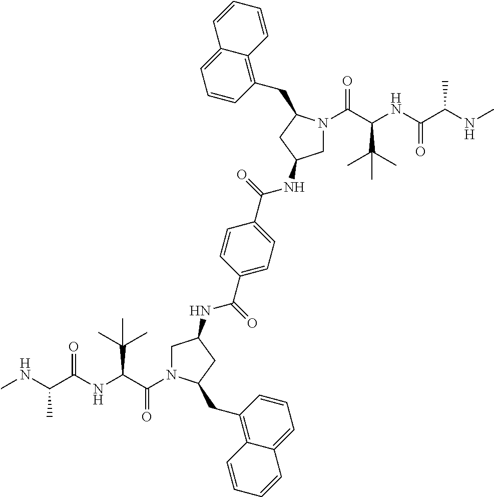 |
| 31 | 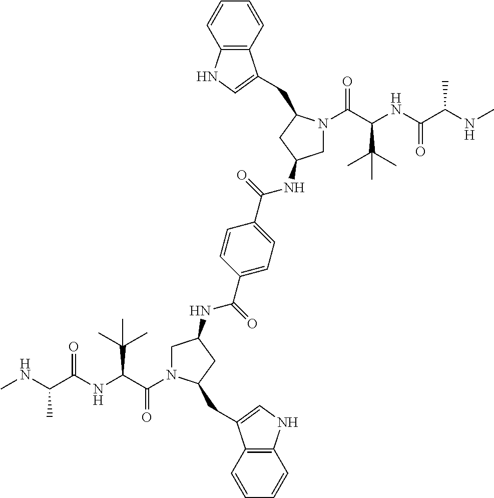 |
| 32 | 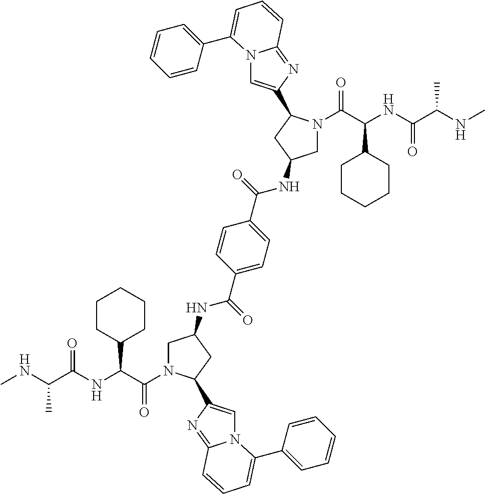 |
| 33 | 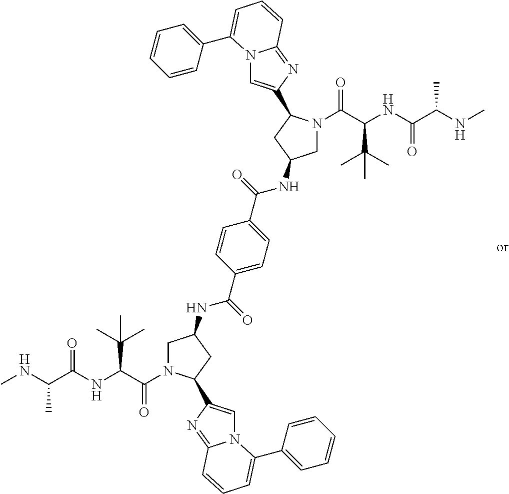 |
| 34 | 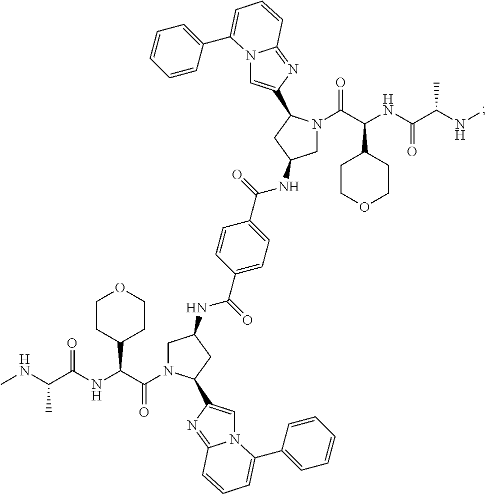 |

or a pharmaceutically acceptable salt thereof.

11. (canceled)

12. A pharmaceutical composition comprising a compound of claim 1, and a pharmaceutically acceptable carrier, diluent or excipient.

13. The pharmaceutical composition of claim 12 further comprising one or more death receptor agonists.

14. The pharmaceutical composition of claim 13, wherein the pharmaceutical composition comprises TRAIL or an anti-TRAIL receptor antibody.

15. The pharmaceutical composition of claim 12 further comprising a therapeutic agent that increases the response of one or more death receptor agonists.

16. The pharmaceutical composition of claim 12 further comprising a chemotherapeutic agent.

17.-19. (canceled)

20. A method of treating a proliferative disease or a disease state characterized by insufficient apoptosis, the method comprising administering to a subject in need thereof a therapeutically effective amount of a compound of claim 1 or a pharmaceutical composition comprising a compound of claim 1, so as to treat the proliferative disease or disease state characterized by insufficient apoptosis.

21. The method of claim 20, wherein the proliferative disease or disease state characterized by insufficient apoptosis is cancer.

22. The method of claim 20, wherein the compound or pharmaceutical composition is administered in combination, simultaneously or sequentially, with:
a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-γ agonist,
m) a PPAR-δ agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug,
s) a proteasome inhibitor,
t) an HDAC inhibitor,
u) an inhibitor of the chymotrypsin-like activity in the proteasome,
v) E3 ligase inhibitors,
w) a modulator of the immune system, or
z) radiation therapy,
so as to treat the cancer.

23. The method of claim 21, further comprising administering to the subject a therapeutically effective amount of a chemotherapeutic agent prior to, simultaneously with or after administration of the compound or pharmaceutical composition.

24. The method of claim 20, wherein the proliferative disease or disease state characterized by insufficient apoptosis is rheumatoid arthritis.

25. The method of claim 24, wherein the compound or pharmaceutical composition is administered in combination, simultaneously or sequentially, with a non-steroidal anti-inflammatory drug (NSAID), analgesic, corticosteroid, or antirheumatic.

26. The method of claim 20, wherein the compound or pharmaceutical composition is administered in combination, simultaneously or sequentially, with a tumor necrosis factor inhibitor, a T-cell costimulatory blocking agent, a B cell depleting agent, an Interleukin-1 (IL-1) receptor antagonist, a p38 inhibitor, a JAK inhibitor, an anti-CD20 MAb, or an anti-IL/ILR agent.

27. The method of claim 20, wherein the compound or pharmaceutical composition is administered in combination, simultaneously or sequentially, with tocilizumab, hydroxychloroquine, sulfasalazine, leflunomide, etanercept, adalimumab, infliximab, rituximab, anakinra, intramuscular gold, azathioprine, cyclophosphamide, cyclosporine A, methotrexate, alemtuzumab, anti-RANKL MAb (denosumab), anti-Blys MAb, belimumab, certolizumab pegol, toclizumab, IL-4, IL-13, or IL-23.

28. The method of claim 20, further comprising administering to the subject a therapeutically effective amount of a death receptor agonist prior to, simultaneously with, or after administration of the compound or pharmaceutical composition, wherein the death receptor agonist is TRAIL or an anti-TRAIL receptor antibody.

29. A probe comprising a compound of claim 1 labeled with a detectable label or an affinity tag.

30.-31. (canceled)

32. A method of modulating TAP function, the method comprising contacting a cell with a compound according to claim 1 so as to prevent binding of a BIR binding protein to an IAP BIR domain, thereby modulating the TAP function.

33. (canceled)
